SEQUENCE LISTING (21 sequences)

SEQ ID NO: 1 - DNA, 40 bp, synthetic construct
  source 1..40: mol_type = other DNA
ctctttccctacacgacgctcttccgatctnnnnnnctag

SEQ ID NO: 2 - DNA, 24 bp, synthetic construct
  source 1..24: mol_type = other DNA
caagcagaagacggcatacgagat

SEQ ID NO: 3 - DNA, 58 bp, synthetic construct
  source 1..58: mol_type = other DNA
gtgactggagttcagacgtgtgctcttccgatcactgctagctagatgactaaacgcg

SEQ ID NO: 4 - DNA, 29 bp, synthetic construct
  source 1..29: mol_type = other DNA
aatgatacggcgaccaccgagatctacac

SEQ ID NO: 5 - DNA, 54 bp, synthetic construct
  source 1..54: mol_type = other DNA
acactctttccctacacgacgctcttccgatctgtggtctggatccaccggtcc

SEQ ID NO: 6 - DNA, 63 bp, synthetic construct
  source 1..63: mol_type = other DNA
ctctttccctacacgacgctcttccgatctnnnnnnnnnnntggtctggatccaccggtccgg

SEQ ID NO: 7 - DNA, 24 bp, synthetic construct
  source 1..24: mol_type = other DNA
caagcagaagacggcatacgagat

SEQ ID NO: 8 - DNA, 56 bp, synthetic construct
  source 1..56: mol_type = other DNA
gtgactggagttcagacgtgtgctcttccgatcctgctagctagatgactaaacgc

SEQ ID NO: 9 - DNA, 24 bp, synthetic construct
  source 1..24: mol_type = other DNA
caagcagaagacggcatacgagat

SEQ ID NO: 10 - DNA, 54 bp, synthetic construct
  source 1..54: mol_type = other DNA
gtgactggagttcagacgtgtgctcttccgatcttacccgtcattggctgtcca

SEQ ID NO: 11 - DNA, 40 bp, synthetic construct
  source 1..40: mol_type = other DNA
aaaaaaaaaaaaaaaaaaaagttgttcttgttgtttcttt

SEQ ID NO: 12 - DNA, 40 bp, synthetic construct; identical to SEQ ID NO: 1
  source 1..40: mol_type = other DNA

SEQ ID NO: 13 - DNA, 24 bp, synthetic construct
  source 1..24: mol_type = other DNA
caagcagaagacggcatacgagat

SEQ ID NO: 14 - DNA, 58 bp, synthetic construct; identical to SEQ ID NO: 3
  source 1..58: mol_type = other DNA

SEQ ID NO: 15 - DNA, 29 bp, synthetic construct
  source 1..29: mol_type = other DNA
aatgatacggcgaccaccgagatctacac

SEQ ID NO: 16 - DNA, 54 bp, synthetic construct; identical to SEQ ID NO: 5
  source 1..54: mol_type = other DNA

SEQ ID NO: 17 - DNA, 63 bp, synthetic construct; identical to SEQ ID NO: 6
  source 1..63: mol_type = other DNA

SEQ ID NO: 18 - DNA, 24 bp, synthetic construct
  source 1..24: mol_type = other DNA
caagcagaagacggcatacgagat

SEQ ID NO: 19 - DNA, 56 bp, synthetic construct; identical to SEQ ID NO: 8
  source 1..56: mol_type = other DNA

SEQ ID NO: 20 - DNA, 24 bp, synthetic construct
  source 1..24: mol_type = other DNA
caagcagaagacggcatacgagat

SEQ ID NO: 21 - DNA, 54 bp, synthetic construct; identical to SEQ ID NO: 10
  source 1..54: mol_type = other DNA

(19) United States
(12) Patent Application Publication
(10) Pub. No.: US 2026/0155212 A1
(43) Pub. Date: Jun. 4, 2026

(54) METHODS AND SYSTEMS FOR PREDICTING CELL-TYPE-SPECIFIC ACTIVITY OF ONE OR MORE UNTRANSLATED REGION RNA SEQUENCES

(71) Applicant: The Regents of the University of California, Oakland, CA (US)

(72) Inventors: Hani Goodarzi, San Francisco, CA (US); Matvei Khoroshkin, San Francisco, CA (US)

(21) Appl. No.: 19/365,119

(22) Filed: Oct. 21, 2025

Related U.S. Application Data
(60) Provisional application No. 63/727,609, filed on Dec. 3, 2024.

(51) Int. Cl. G16B 40/30; A61K 31/7105; C07H 21/02; G16B 30/00

(52) U.S. Cl. CPC G16B 40/30; A61K 31/7105; C07H 21/02; G16B 30/00

(57) ABSTRACT

This disclosure provides methods and systems generally relating to predicting cell-type-specific activity of one or more untranslated region (UTR) RNA sequences and generating UTR RNA sequences with predefined cell-type-specific activity, in particular using machine learning models.

### 1. CROSS REFERENCE TO RELATED APPLICATIONS

The present disclosure claims priority to U.S. Provisional Patent Application No. 63/727,609 filed Dec. 3, 2024, which is hereby incorporated by reference in its entirety.

### 2. GOVERNMENT SUPPORT CLAUSE

This invention was made with government support under R01 CA244634 awarded by the National Institutes of Health. The government has certain rights in the invention.

### 3. REFERENCE TO SEQUENCE LISTING SUBMITTED ELECTRONICALLY

This application contains a Sequence Listing which has been submitted electronically in XML format and is hereby incorporated by reference in its entirety. The XML file, named 061818-5533-US, was created on Mar. 20, 2026 and is 19.5 kilobytes in size.

### 4. TECHNICAL FIELD

This specification describes technologies generally relating to predicting cell-type-specific activity of one or more untranslated region (UTR) RNA sequences and generating UTR RNA sequences with predefined cell-type-specific activity, in particular using machine learning models.

### 5. BACKGROUND

mRNA therapeutics are revolutionizing disease treatment by leveraging messenger RNA (mRNA) to direct cells to produce therapeutic proteins, offering capabilities beyond those of traditional small molecules (Rohner et al. 2022). This approach provides a versatile platform for addressing various unmet medical needs. Recent advances have also highlighted the role of regulatory RNA components in controlling post-transcriptional processes. For example, WO 2024/102773, incorporated herein by reference in its entirety, describes integrative computational and experimental methods for systematically predicting and identifying riboswitches. However, despite significant advancements, current mRNA therapeutics face critical challenges, particularly in achieving stability and cell type specificity of therapeutic mRNAs. Rapid degradation of mRNA molecules compromises their efficacy (Metkar, Pepin, and Moore 2024), while insufficient cell type specificity may produce off-target effects, causing toxicity in non-relevant tissues (Balmayor 2022).

The cell type-specificity at the transcriptional level has been extensively studied and successfully achieved, aiding the development of DNA-based therapies (Ong and Corces 2011; H. Wu et al. 2014). Yet, the post-transcriptional regulation, crucial for mRNA therapeutics, remains less explored (Waldman et al. 2010; Bateman et al. 2003; Obernosterer et al. 2006) and designing mRNAs with cell type-specific expression remains particularly challenging (Kim et al. 2024; Leppek et al. 2022). One common strategy involves incorporating microRNA binding sites into 3′ untranslated region (3′UTR), utilizing cell type-specific microRNAs to selectively suppress mRNA activity (Jain et al. 2018). However, this approach had limited success, as it struggled to generalize across cell types due to the variability in microRNA expression (Ludwig et al. 2016) and activity.

A critical hurdle in mRNA design lies in the lack of large-scale, consistent datasets to study post-transcriptional regulation across different cell types. Most machine learning models aimed at enhancing RNA function either rely solely on RNA sequence data or are trained on measurements from a single cell line, overlooking cell type heterogeneity (Riley, Robson, and Green 2023; Chu et al. 2024; Barazandeh et al. 2023; Zhang et al. 2023). The few models that do account for cell type specificity often suffer from dataset heterogeneity and biases introduced by varying experimental protocols, which hinder their ability to generalize across diverse cell types. For example, ribosome profiling (Ribo-Seq) offers a valuable method for assessing ribosome occupancy, yet these measurements are influenced not only by the mRNA sequence but also by the genomic context of individual genes (Papadopoulos et al. 2024), introducing undesired variance and complicating the identification of regulatory signals within the sequence. Furthermore, large-scale collections of Ribo-Seq data span datasets collected across different laboratories using various non-standardized protocols, contributing to additional heterogeneity (Yue Liu et al. 2024). Although deep learning has shown promise in related areas, such as designing transcriptional enhancers (de Almeida et al. 2024; Yin et al. 2024; Gosai et al. 2023), the absence of suitable data has limited its application to cell type-specific mRNA design (Castillo-Hair et al. 2024).

While many datasets measuring static RNA levels using RNA-Seq are available, these datasets are often confounded by transcriptional activity (Yansheng Liu, Beyer, and Aebersold 2016), making it difficult to discern the sequence determinants of post-transcriptional regulation.

### 6. SUMMARY

In general, there is a need for systems and methods for screening RNA sequences, such as genomic sequences, transcript, pre-mRNAs, mRNAs, splice variants, etc., for untranslated region (UTR) sequences with enhanced cell type-specificity for a given application. Additionally, there is a need for systems and methods for performing a deo novo design of UTR RNA sequences that are likely to have target properties for a given application and for using generative processes for sequence design and selection based on target properties, including input optimization processes. Given the above background, there is a need in the art for improved methods and systems for determining UTR RNA sequences with cell-type-specific activity, such as enhanced cell type-specific mRNA stability and/or translation. Provided herein, among other aspects, are machine learning approaches to evaluating, predicting, and/or designing UTR RNA sequences using a model, e.g., a model including one or more encoder blocks, one or more decoder blocks, and/or where all or a portion of the model is pretrained.

One aspect of the present disclosure provides a method for optimizing a model to predict cell-type-specific activity of one or more UTR RNA sequences. In some embodiments, the method is performed at a computer system comprising at least one processor and a memory storing at least one program for execution by the at least one processor. In some embodiments, the method includes obtaining a model comprising a plurality of parameters across a first block and a second block, where each of the first block and the second block comprises an attention mechanism, the plurality of parameters reflects, at least in part, pretraining information for a plurality of pretraining samples comprising, for each respective pretraining sample in the plurality of pretraining samples, a corresponding unlabeled UTR RNA sequence, and the model generates, responsive to inputting first test information comprising a respective RNA sequence to the model, an indication of an activity associated with the UTR RNA sequence in each target cell type in a plurality of target cell types, or a representation thereof. In some embodiments, the method includes retraining the model using a plurality of training samples, where each respective training sample in the plurality of training samples comprises training information including a corresponding training UTR RNA sequence and a corresponding training set of one or more metrics for an activity associated with the UTR RNA sequence in each target cell type in a plurality of target cell types, such as the cell-type-specific activity and/or the delta activity for each target cell type in the plurality of target cell types; thereby updating the plurality of parameters.

In some embodiments, the method further includes obtaining, in electronic form, a second test information comprising an UTR RNA sequence, and inputting the second test information into the retrained model, where the retrained model applies the updated plurality of parameters to the second test information to generate, as output from the retrained model, a test set of one or more metrics for the cell-type-specific activity and/or the delta activity for each target cell type in the plurality of target cell types. In some embodiments, the method is applied repeatedly to a plurality of UTR RNA sequences to predict the activity for each respective UTR RNA sequence in the plurality of UTR RNA sequences.

In another aspect, the present disclosure provides a method for predicting cell-type-specific activity of an untranslated region (UTR) RNA sequence, comprising: at a computer system comprising at least one processor and a memory storing at least one program for execution by the at least one processor:
A) obtaining UTR RNA sequence data corresponding to a plurality of UTR RNA sequences;
B) processing the UTR RNA sequence data using a model, wherein the model is configured to, for each respective UTR RNA sequence in the plurality of UTR RNA sequences:
   predict a cell-type-specific activity of the respective UTR RNA sequence for each target cell type in a plurality of target cell types, and/or
   predict a delta activity of the UTR RNA sequence for each target cell type in the plurality of target cell types, wherein the delta activity is defined as difference between the predicted cell-type-specific activity specific to the respective target cell type and an average activity across the plurality of target cell types; and
C) outputting, for each respective UTR RNA sequence in the plurality of UTR RNA sequences, a predicted set of metrics for the cell-type-specific activity and/or the delta activity for each target cell type in the plurality of target cell types.

In some embodiments, the cell-type-specific activity of an UTR RNA sequence quantifies effect of the UTR RNA sequence on translation and/or mRNA stability of a target gene under suitable conditions, when the UTR RNA sequence and the target gene are operably linked in an expression construct.

In some embodiments, the method further comprises validating the outputted sets of metrics with one or more in vitro or in vivo assays.

In some embodiments, the method further comprises feeding validation data obtained from the one or more in vitro or in vivo assays back to the model, thereby improving accuracy of prediction by the model.

In some embodiments, the method further comprises validating the outputted sets of metrics with a Massively Parallel Reporter Assay (MPRA).

In some embodiments, the model is trained with a Massively Parallel Reporter Assay (MPRA) dataset to predict cell-type-specific activity and delta activity of RNA sequences corresponding to one or more target cell types, wherein the MPRA dataset comprises:
the plurality of UTR RNA sequences, and
for each respective UTR RNA in the plurality of UTR RNA sequences, measurements of corresponding cell-type-specific activity specific to the one or more target cell types, measured from the MPRA.

In some embodiments, the MPRA dataset provides additional information for each respective UTR RNA sequence in the plurality of UTR RNA sequences, comprising one or more of triple phase information, sequence motifs, and structural features.

In some embodiments, the plurality of UTR RNA sequences in the MPRA dataset comprises 5′ UTR RNA sequences, 3′ UTR RNA sequences, or both.

In some embodiments, the plurality of UTR RNA sequences in the MPRA dataset comprises at least 1,000, at least 10,000, at least 100,000, or at least 1×10⁶ 5′ UTR RNA sequences.

In some embodiments, the plurality of UTR RNA sequences in the MPRA dataset comprises at least 1,000, at least 10,000, at least 100,000, or at least 1×10⁶ 3′ UTR RNA sequences.

In some embodiments, each UTR RNA sequence in the plurality of the UTR RNA sequences comprises at most 50, at most 100, at most 150, at most 200, at most 250, at most 300, at most 350, at most 400, at most 450, or at most 500 nucleotides.

In some embodiments, step A) further comprises obtaining for each respective UTR RNA sequence in the plurality of UTR RNA sequences, additional sequence information comprising triplet phase information for each respective UTR RNA sequence. In some embodiments, the triplet phase information improves accuracy of prediction by the model.

In some embodiments, specific to each target cell type in the plurality of target cell types, the outputted set of metrics comprises:
a cell type specificity index (τ), for each respective UTR RNA sequence in the plurality of UTR RNA sequences, wherein τ:
   ranges from 0 to 1,
      indicates ubiquitous activity at 0, and
      indicates exclusive activity in a single cell type at 1; and
a delta activity value (A) quantifying difference between the predicted cell-type-specific activity and an average activity across the plurality of target cell types.

In some embodiments, the cell type specificity index (τ) and the delta activity value (Δ) are used to assign a classification score to each respective UTR RNA sequence in the plurality of UTR RNA sequences, wherein the classification score reflects a ranked continuum of specificity, ranging from highly ubiquitous to highly cell-type-specific.

In some embodiments, the model disclosed herein comprises:
an encoder block that attends to a representation of each respective UTR RNA sequence in the plurality of UTR RNA sequences to generate one or more embeddings; and
a decoder block that attends to the one or more embeddings for each respective UTR RNA sequence to generate a predicted set of metrics corresponding to each target cell type in the plurality of target cell types.

In some embodiments, the encoder block attends to additional sequence information and the UTR RNA sequence data to generate one or more embeddings representing each respective UTR RNA sequence in the plurality of UTR RNA sequences and its annotations.

In some embodiments, the decoder block attends to the one or more embeddings generated by the encoder block to refine the embeddings and predict the cell-type-specific activity and delta activity for each respective UTR RNA sequence in the plurality of UTR RNA sequences.

In some embodiments, the encoder block is configured to:
process the representation of each respective UTR RNA sequence in the plurality of UTR RNA sequences through a sequence of computational operations, each computational operation comprising:
   (i) a convolutional operation,
   (ii) an activation function,
   (iii) a normalization operation,
   (iv) a feature recalibration operation, and
   (v) a residual connection that combines outputs with its input; and
generate one or more embeddings representing the representation of each respective UTR RNA sequence in the plurality of UTR RNA sequences; and

In some embodiments, the decoder block is configured to:
process the one or more embeddings generated by the encoder block through additional computational operations to refine representations of each respective UTR RNA sequence in the plurality of UTR RNA sequences; and
produce for each respective UTR RNA sequence in the plurality of UTR RNA sequences, a predicted set of metrics for the cell-type-specific activity and/or the delta activity for each target cell type in a plurality of target cell types.

In some embodiments, the model comprises a LegNet model.

In some embodiments, the present disclosure provides herein a method for regulating expression of a target gene in a target cell, comprising performing the method disclosed above, and further comprising operably linking an UTR RNA sequence to the target gene in an expression construct, wherein the UTR RNA sequence exhibits cell-type-specific activity when the expression construct is transferred to a corresponding target cell type and under suitable conditions; and transferring the expression construct to the target cell.

Another aspect of the present disclosure provides a method for predicting regulatory motifs in an UTR RNA sequence, comprising:
at a computer system comprising at least one processor and a memory storing at least one program for execution by the at least one processor:
A) obtaining UTR RNA sequence data for each respective UTR RNA sequence in a plurality of UTR RNA sequences, and associated activity for each respective UTR RNA sequence;
B) processing the UTR RNA sequence data using a model, wherein the model is configured to, for each respective UTR RNA sequence in the plurality of UTR RNA sequences, identify one or more candidate motifs in each respective UTR RNA sequence;
C) determining, for each respective UTR RNA sequence in the plurality of UTR RNA sequences, correlation of each candidate motif identified sequences with the associated UTR RNA activity; and
D) outputting a ranked list of candidate motifs identified in the plurality of UTR RNA sequences, wherein the ranking is based on correlation between each respective candidate motif and UTR activity.

In some embodiments, the ranking in step D) is based on the correlation between each respective candidate motif and the UTR RNA activity of the respective UTR RNA sequence.

In some embodiments, the ranking in step D) is based on the correlation between each respective candidate motif and aggregated UTR RNA activity across UTR RNA sequences comprising the respective candidate motif.

In some embodiments, the model is configured to further:
predict an activity for each respective UTR RNA sequence in the plurality of UTR RNA sequences, and
analyze each respective UTR RNA sequence to identify the one or more candidate motifs in each respective UTR RNA sequence that are associated with the predicted activity.

In some embodiments, step B) further comprises generating an importance score for each candidate motif in each respective UTR RNA sequence.

In some embodiments, step C) comprises:
correlating each candidate motif in each respective UTR RNA sequence in the plurality of UTR RNA sequences with experimentally measured activity data of the respective UTR RNA sequence to assign a partial correlation score for each candidate motif, wherein the partial correlation score quantifies contribution of the candidate motif to UTR RNA activity of each respective UTR RNA sequence; and
aggregating the partial correlation scores across the plurality of UTR RNA sequences to assign a mean correlation score for each candidate motif, wherein the mean correlation score quantifies average contribution of the candidate motif to UTR RNA activity of UTR RNA sequences comprising the respective motif.

In some embodiments, the outputting in step D) comprises outputting a ranked list of candidate motifs based on their respective mean correlation scores.

In some embodiments, for each respective UTR RNA sequence in the plurality of UTR RNA sequences, the associated activity includes cell-type-specific activity specific to each target cell type in a plurality of target cell types.

In some embodiments, the method further comprises validating the outputted ranked list of candidate motifs with one or more in vitro or in vivo assays.

In some embodiments, the method further comprises feeding validation data obtained from the one or more in vitro or in vivo assays back to the model, thereby improving accuracy of prediction by the model.

In some embodiments, the method further comprises regulating expression of a target gene in a target cell, comprising:
operably linking an UTR RNA sequence comprising one or more candidate motifs to the target gene in an expression construct, wherein the UTR RNA sequence exhibits cell-type-specific activity when the expression construct is transferred to a corresponding target cell type and under suitable conditions; and
transferring the expression construct to the target cell.

Yet another aspect of the present disclosure provides a method for generating a plurality of UTR RNA sequences with predefined cell-type-specific activity, comprising:
at a computer system comprising at least one processor and a memory storing at least one program for execution by the at least one processor:
A) obtaining input data comprising one or more predefined activity and one or more cell type-specificity constraints;
B) processing the input data using a first portion of a model to generate a plurality of UTR RNA sequences, wherein the first portion of the model is configured to refine the UTR RNA sequences based on the one or more predefined activity and the one or more cell type-specificity constraints;
C) responsive to step B), using a second portion of the model to predict, for each respective UTR RNA sequence in the plurality of UTR RNA sequences generated in step B):
   a cell-type-specific activity of the respective UTR RNA sequence for each target cell type in a plurality of target cell types, and/or
   a delta activity of the respective UTR RNA sequence for each target cell type in the plurality of target cell types, wherein the delta activity is defined as difference between the predicted cell-type-specific activity specific to the respective target cell type and an average activity across the plurality of target cell types;
D) generating, for each respective UTR RNA sequence in the plurality of UTR RNA sequences generated in step B), a predicted set of metrics for the cell-type-specific activity and/or the delta activity for each target cell type in the plurality of target cell types;
E) selecting a sub-plurality of UTR RNA sequences from the plurality of UTR RNA sequences generated in step B), wherein the corresponding predicted sets of metrics for the sub-plurality of UTR RNA sequences satisfy the one or more predefined activity and the one or more cell type-specificity constraints; and
F) outputting the selected sub-plurality of UTR RNA sequences.

In some embodiments, the method further comprises:
evaluating each respective UTR RNA sequence in the plurality of UTR RNA sequences generated to calculate a cell type activity difference (CTAD) score, wherein the CTAD score quantifies the difference in predicted activity of a UTR RNA sequence between two target cell types, and
selecting UTR RNA sequences that maximize the CTAD score while satisfying the one or more predefined activity and the one or more cell type-specificity constraints.

In some embodiments, the second portion of the model outputs the predicted sets of metrics for the cell-type-specific activity and/or the delta activity in step D); and
wherein following step D) and prior to step E), the method repeats steps B)-D), comprising:
   feeding the predicted sets of metrics generated in step D) back to the first portion of the model, thereby generating UTR RNA sequences with refined metrics through steps B)-D) in subsequent iterations.

In some embodiments, the first portion of the model comprises:
an encoder block configured to process the one or more predefined activity and the one or more cell type-specificity constraints, and
a decoder block configured to iteratively generate UTR RNA sequences based on output of the encoder block and the one or more predefined activity and the one or more cell type-specificity constraints.

In some embodiments, the model comprises a cold diffusion model, a genetic algorithm, a random sampling model, or a combination thereof.

In some embodiments, the model comprises a cold diffusion model configured to generate UTR RNA sequences through a plurality of iterations, each iteration comprising introducing one or more mutations to the UTR RNA sequences, based on the one or more predefined activity and the one or more cell type-specificity constraints.

In some embodiments, the model comprises a genetic algorithm model configured to generate UTR RNA sequences through a plurality of iterations, each iteration comprising one or more of selection, mutation, and crossover, based on the one or more predefined activity and the one or more cell type-specificity constraints.

In some embodiments, the model comprises a motif-based design algorithm.

In some embodiments, wherein the encoder block is configured to generate a latent feature representation of the one or more predefined activity and the one or more cell type-specificity constraints, and the decoder block is configured to use the latent feature representation to generate UTR RNA sequences.

In some embodiments, wherein the method further comprises validating the sub-plurality of UTR RNA sequences with one or more in vitro or in vivo assays.

In some embodiments, wherein the method further comprises feeding validation data obtained from the one or more in vitro or in vivo assays back to the model, thereby improving accuracy of prediction by the model.

In some embodiments, one or more UTR RNA sequences in the selected sub-plurality of UTR RNA sequences have cell-type-specific activity in blood cells, colon cells, ovarian cells, breast tissue cells, liver tissue cells, or a combination thereof.

In some embodiments, one or more UTR RNA sequences in the selected sub-plurality of UTR RNA sequences have cell-type-specific activity in Jurkat and Nalm-6 (blood tissue), SW-480 (colon tissue), PA-1 (ovarian tissue), MDA-MB-231 (breast tissue), HepG2 (liver tissue), or a combination thereof.

Yet another aspect of the present disclosure provides a system comprising:
a processor; and
a memory storing instructions, when executed by the processor, cause the processor to perform the method in accordance with the embodiments of the present disclosure.

In some embodiments, the present disclosure provides a non-transitory computer-readable medium storing computer code comprising instructions, when executed by one or more processors, causing the processors to perform the method in accordance with the embodiments of the present disclosure.

In another aspect, the present disclosure provides an UTR RNA sequence having cell-type-specific activity, obtained according to the method in accordance with the embodiments of the present disclosure.

In some embodiments, the present disclosure provides an UTR RNA sequence having cell-type-specific activity, obtained according to a method comprising:
at a computer system comprising at least one processor and a memory storing at least one program for execution by the at least one processor:
A) obtaining input data comprising one or more predefined activity and one or more cell type-specificity constraints;
B) processing the input data using a first portion of a model to generate a plurality of UTR RNA sequences, wherein the first portion of the model is configured to refine the UTR RNA sequences based on the one or more predefined activity and the one or more cell type-specificity constraints;
C) responsive to step B), using a second portion of the model to predict, for each respective UTR RNA sequence in the plurality of UTR RNA sequences generated in step B):
   a cell-type-specific activity of the respective UTR RNA sequence for each target cell type in a plurality of target cell types, and/or
   a delta activity of the respective UTR RNA sequence for each target cell type in the plurality of target cell types, wherein the delta activity is defined as difference between the predicted cell-type-specific activity specific to the respective target cell type and an average activity across the plurality of target cell types;
D) generating, for each respective UTR RNA sequence in the plurality of UTR RNA sequences generated in step B), a predicted set of metrics for the cell-type-specific activity and/or the delta activity for each target cell type in the plurality of target cell types;
E) selecting a sub-plurality of UTR RNA sequences from the plurality of UTR RNA sequences generated in step B), wherein the corresponding predicted sets of metrics for the sub-plurality of UTR RNA sequences satisfy the one or more predefined activity and the one or more cell type-specificity constraints; and
F) outputting the selected sub-plurality of UTR RNA sequences.

In some embodiments, the UTR RNA sequence of the present invention is obtained in accordance with the method disclosed herein.

In some embodiments, the present disclosure provides an UTR RNA sequence having cell-type-specific activity, wherein the UTR RNA sequence represents a regulatory motif, obtained according to a method comprising:
at a computer system comprising at least one processor and a memory storing at least one program for execution by the at least one processor:
A) obtaining UTR RNA sequence data for each respective UTR RNA sequence in a plurality of UTR RNA sequences, and associated activity for each respective UTR RNA sequence;
B) processing the UTR RNA sequence data using a model, wherein the model is configured to, for each respective UTR RNA sequence in the plurality of UTR RNA sequences, identify one or more candidate motifs in each respective UTR RNA sequence;
C) determining, for each respective UTR RNA sequence in the plurality of UTR RNA sequences, correlation of each candidate motif identified sequences with the associated UTR RNA activity; and
D) outputting a ranked list of candidate motifs identified in the plurality of UTR RNA sequences, wherein the ranking is based on correlation between each respective candidate motif and UTR activity.

In some embodiments, the method for obtaining the UTR RNA sequence further comprises one or more features in accordance with the present embodiments disclosed herein.

In some embodiments, the present disclosure provides an UTR RNA sequence having cell-type-specific activity, wherein the UTR RNA sequence is generated from a system comprising:
a processor; and
a memory storing instructions, when executed by the processor, cause the processor to perform steps comprising the method in accordance with the present embodiments disclosed herein.

In some embodiments, the present disclosure provides an UTR RNA sequence having cell-type-specific activity, wherein the UTR RNA sequence is generated according to instructions stored in a non-transitory computer-readable medium, when executed by one or more processors, causing the processors to perform the method in accordance with the present embodiments disclosed herein.

In some embodiments, the cell-type-specific activity of the respective UTR RNA sequence quantifies effect of the UTR RNA sequence on translation and/or mRNA stability of a target gene under suitable conditions, when the UTR RNA sequence and the target gene are operably linked in an expression construct.

In some embodiments, the UTR RNA sequence increases the expression of a target gene operably linked to the UTR RNA sequence in one or more target cell types under suitable conditions. In some embodiments, the UTR RNA sequence increases mRNA stability of a target gene operably linked to the UTR RNA sequence in one or more target cell types under suitable conditions.

In some embodiments, the UTR RNA sequence compress a 5′ UTR RNA sequence. In some embodiments, the UTR RNA sequence compress a 3′ UTR RNA sequence.

In some embodiments, the UTR RNA sequence exhibits a cell type specificity index (τ) of at least 0.1, at least 0.2, at least 0.3, at least 0.4, at least 0.5, at least 0.6, at least 0.7, at least 0.8, or at least 0.9; wherein τ:
ranges from 0 to 1,
indicates ubiquitous activity at 0, and
indicates In some embodiments, the UTR RNA sequence exhibits a delta activity value (Δ) corresponding to a specific target cell type that is at least 10%, at least 20%, at least 30%, at least 40%, at least 50%, at least 60%, at least 70%, at least 80%, or at least 90% of an average activity across a plurality of target cell types, wherein A quantifies difference between the cell-type-specific activity corresponding to the specific target cell type and the average activity across the plurality of target cell types. In some embodiments, the cell-type-specific activity is measured by a Massively Parallel Reporter Assay (MPRA).

In some embodiments, the UTR RNA sequence has cell-type-specific activity in at most 1, at most 2, at most 3, at most 4, at most 5, at most 6, at most 7, at most 8, at most 9, at most 10, at most 20, at most 30, at most 40, at most 50, at most 60, at most 70, at most 80, at most 90, or at most 100 target cell types.

In some embodiments, the UTR RNA sequence does not have cell-type-specific activity in at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, or at least 100 target cell types.

In some embodiments, the UTR RNA sequence has cell-type-specific activity in immune cells, blood cells, colon cells, ovarian cells, breast tissue cells, liver tissue cells, or a combination thereof.

In some embodiments, the UTR RNA sequence has cell-type-specific activity in Jurkat and Nalm-6 (blood tissue), SW-480 (colon tissue), PA-1 (ovarian tissue), MDA-MB-231 (breast tissue), HepG2 (liver tissue), or a combination thereof.

In some embodiments, the UTR RNA sequence increases the expression of a target gene operably linked to the UTR RNA sequence in T cells under suitable conditions.

Another aspect of the present disclosure provides an expression construct, comprising an UTR RNA sequence that has cell-type-specific activity specific to a target cell type, and a target gene, wherein the UTR RNA sequence is operably linked to the target gene.

Yet another aspect of the present disclosure provides an expression construct, comprising an UTR RNA sequence that has cell-type-specific activity specific to a target cell type, and a target gene, wherein the UTR RNA sequence is operably linked to the target gene, and wherein the UTR RNA sequence is obtained according to the method disclosed herein.

In another aspect, the present disclosure provides an expression construct, comprising an UTR RNA sequence in accordance with the embodiments disclosed herein, and a target gene, wherein the UTR RNA sequence is operably linked to the target gene.

In another aspect, the present disclosure provides a pharmaceutical composition comprising an expression construct, wherein the expression construct comprises the UTR RNA sequence that has cell-type-specific activity specific to a target cell type and is obtained according to the method disclosed herein, and a target gene, wherein the UTR RNA sequence is operably linked to the target gene.

In some embodiments, the UTR RNA sequence increases the expression of the target gene when the expression construct is transferred into the target cell and under suitable conditions.

In some embodiments, the UTR RNA sequence increases mRNA stability of the target gene when the expression construct is transferred into the target cell and under suitable conditions.

In some embodiments, the target gene encodes CYP2E1, and the target cell type is T cell.

In some embodiments, the target gene encodes PTEN.

Another aspect of the present disclosure provides a pharmaceutical formulation, comprising the UTR RNA sequence disclosed herein.

Still another aspect of the present disclosure provides a pharmaceutical formulation, comprising the expression construct disclosed herein.

In another aspect, the present disclosure provides a method for regulating expression of a target gene in a target cell, comprising:
obtaining an expression construct disclosed herein; and
transferring the expression construct into the target cell.

In another aspect, the present disclosure provides a method for regulating expression of a target gene in a target cell, the method comprising administering to the target cell the pharmaceutical formulation comprising the expression construct in accordance with the embodiments disclosed herein, thereby inducing expression and/or modulating stability of the target gene in the target cell under suitable conditions.

Still another aspect of the present disclosure provides a computer system including one or more processors and a non-transitory computer-readable medium including computer-executable instructions that, when executed by the one or more processors, cause the processors to perform any of the methods and/or embodiments disclosed above.

Yet another aspect of the present disclosure provides a non-transitory computer-readable storage medium having stored thereon program code instructions that, when executed by a processor, cause the processor to perform any of the methods and/or embodiments disclosed above.

The systems, methods, and non-transitory computer readable storage medium of the present invention have other features and advantages that will be apparent from, or are set forth in more detail in, the accompanying drawings, which are incorporated herein, and the following Detailed Description, which together serve to explain certain principles of exemplary embodiments of the present invention.

### 7. BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-C collectively illustrate an example computer system for predicting cell-type-specific activity of an untranslated region (UTR) RNA sequence and/or optimizing a model to predict the same, in accordance with various embodiments of the present disclosure.

FIGS. 2A-E illustrate massively parallel reporter assays capture cell type-specific regulatory activity. A. Schematic representation of an MPRA experiment. B. Effect of known biological signals on average activity. The smoothed histograms show the relation between measured average activity and: the presence of uORFs or uAUGs in 5′UTRs (top left), and of dORFs (bottom left), AU-rich elements (top right) or miRNA seed consensus sequences (bottom right) in 3′UTRs. C and D. Visualization of 5′UTR (C) and 3′UTR (D) cell type-specific regulatory pattern space. Each point in the UMAP chart (left) corresponds to the measured cell type-specific activity levels for a single 5′UTR sequence. The point coloring is based on the results of K-means clustering, in which several clusters with distinct captured cell type-specific activity patterns were selected. The activity levels for each of the selected cluster's sequences (right) are shown, colored by cluster. Grey represents all the clusters not highlighted.

FIGS. 3A-B illustrate neural network for activity analysis. A. Schematic representation of the PARADE double regression task. The adapted LegNet neural network was supplied with one-hot encoded RNA UTR sequences, with additional channels representing biologically relevant information: the triplet phase and cell type. The neural network was trained to simultaneously predict two sequence characteristics: cell type-specific activity value and its difference from average activity. B. PARADE prediction's for average activity values. Pearson's correlation coefficients (r) between experimental activity values (x) and PARADE predictions (y) were calculated. In the bottom heatmap table, the comparison of PARADE performance with older methods is shown, with Pearson r shown for every model and cell line type tested.

FIGS. 4A-B illustrate identified regulatory motifs and their correlation with UTR activity in cell-specific contexts A. Waterfall plot of top and bottom 5 sorted by mean partial correlation of SPRy-SARUS motif score with experimentally measured sequence activity of RNA-binding protein motifs detected by FIRE and TF-MoDISco. B. Heatmap of partial correlations of motif score with activity in different cell lines. RBP motif was discovered by FIRE and TF-MoDISco tools and annotated by MACRO-APE software.

FIGS. 5A-D illustrate de novo design of UTR sequences. A. Diffusion generative model implementation. Schematic representation of the procedure applied to a mini-batch of sequences is depicted in the top. In the bottom, the correlations between the target activity are shown for different cell type-specific models. Hexagonal histogram plots of the relation between target activity and predicted activity are shown for SW480 cell line type for 5′UTRs and HepG2 cell line type for 3′UTRs. B. An overview of methods used for sequence generation. C. Visualization of 5′ UTR PARADE Predictor latent space. Two charts depict the PCA-embedded latent space. Kernel density estimation charts are drawn on top for the initial library (top left), diffusion and genetic algorithm generation pools (top right), with the pool of random sequences shown in grey in the background. For 2 cell line types (HepG2 and SW480), sequences with the largest directional difference between predicted activity in these lines were selected (1000 for each direction and generation method). Their position in the model latent space is shown for diffusion-generated sequences (middle left) and ones yielded by the genetic algorithm (middle right). In the bottom charts, the predicted cell line type specific activity for these sequences is shown. D. Similar visualization of 3′ UTR PARADE Predictor latent space. See the description of FIG. 3C for details.

FIGS. 6A-G illustrate generative methods validation in cell lines. A. Relative distribution of the second library sequences in the PARADE Predictor latent space. Similarly to FIG. 3C, the pool of random sequences is shown in grey, with the 1st library shown in contour and 2nd library shown in color (green for 5′UTR and purple for 3′UTR). B. Correlations of PARADE predictions with real activity estimations. The scatter diagrams are shown for each cell line type for 5′UTR (top) and 3′UTR (bottom). C. A box plot for the distributions of the τ (tau) expression specificity metric w.r.t. the method used for sequence generation. Each box represents the distribution of the τ metric for 5′UTRs (left) and 3′UTRs (right) for each of the applied generative methods. Additionally, referential characterized RNA sequences were included in the experiment for comparison, and are shown in a swarm plot. 3′UTRs used in Moderna and Pfizer vaccines are marked on the corresponding chart. D. Significance of the directional generation methods

FIGS. 7A-D further illustrate cell-type-specific activity data obtained from the model.

FIGS. 8A-E further illustrate a flowchart illustrating example methods for predicting cell-type-specific activity of an untranslated region (UTR) RNA sequence, and that the prediction of sequence activity of both 5′ and 3′ UTRs and identification the regulatory elements by the method in accordance with embodiments of the present invention.

FIG. 9 further illustrates identified regulatory motifs and their correlation with UTR activity in cell-specific contexts.

FIG. 10 illustrate example generative models and their workflows.

FIGS. 11A-D illustrate evaluation of engineered RNA sequence activity and specificity.

FIGS. 12A-H further illustrate data obtained from the generative models.

It should be understood that the appended drawings are not necessarily to scale, presenting a somewhat simplified representation of various features illustrative of the basic principles of the invention.

### 8. DETAILED DESCRIPTION 8.1. Introduction

Thus, there is a need in the art for machine learning models that provide the ability to screen many more UTR RNA sequences in silico, compared to in vitro approaches, to perform a de novo design of sequences that enable cell-type-specific regulation of mRNA stability and/or expression, and to use generative processes for guide design and selection based on target properties, including input optimization processes.

### 8.2. Definitions

Unless defined otherwise, all technical and scientific terms used herein have the meaning commonly understood by one of ordinary skill in the art to which the invention pertains.

The terms “screening method” and “screening assay”, used interchangeably herein, refer to a method used for characterizing or selecting compounds such as RNA sequences from a collection of compounds based on the activities of the compounds. influence the readout of choice rather than to tests which test the ability of one compound to influence a readout. In an exemplary embodiment, the subject methods and/or assays identify compounds not previously known to have the effect that is being screened for. In one embodiment, high throughput screening can be used to assay for the activity of a compound.

As used interchangeably herein, the terms “riboswitch” or “RNA switch” refer to a regulatory segment of a messenger RNA (mRNA) molecule binding a small molecule, resulting in a change in production of the protein encoded by the mRNA. Thus, a mRNA containing a riboswitch is directly involved in regulating its own activity, in response to the concentrations of its effector molecule.

The terms “nucleic acid” or “nucleic acid sequence”, “nucleic acid molecule”, “nucleic acid fragment” or “polynucleotide” are used interchangeably herein. A polynucleotide is a biopolymer composed of nucleotide monomers covalently bonded in a chain. DNA (deoxyribonucleic acid) and RNA (ribonucleic acid) are examples of polynucleotides with distinct biological functions. DNA consists of two chains of polynucleotides, with each chain in the form of a helical spiral. RNA is more often found in nature as a single-strand folded onto itself. Exemplary types of RNA include double-stranded RNA (dsRNA), small interfering RNA (siRNA), short hairpin (shRNA), microRNA (miRNA), messenger RNA (mRNA), antisense RNA, transfer RNA (tRNA), small nuclear RNA (snRNA), and ribosomal RNA (rRNA).

In the present disclosure, the term “UTR (untranslated region)” refers to the portion in an mRNA chain not serving as the template of a protein gene. The untranslated portion of mRNA, and generally, upstream of the coding region is referred to as 5′ UTR, and the portion downstream thereof, is referred to as 3′ UTR.

The term “genome,” as used herein, generally refers to genomic information from a subject, which may be, for example, at least a portion or an entirety of a subject's hereditary information. A genome can be encoded either in DNA or in RNA. A genome can comprise coding regions (e.g., that code for proteins) as well as non-coding regions. A genome can include the sequence of all chromosomes together in an organism. For example, the human genome ordinarily has a total of 46 chromosomes. The sequence of all of these together may constitute a human genome.

As used herein the term “transcriptome” refers to the set of all transcripts, such as messenger RNA (mRNA) molecules, small interfering RNA (siRNA) molecules, transfer RNA (tRNA) molecules, ribosomal RNA (rRNA) molecules, in a sample, for example, a single cell or a population of cells. In some embodiments, transcriptome not only refers to the species of transcripts, such as mRNA species, but also the amount of each species in the sample. In some embodiments, a transcriptome includes each mRNA molecule in the sample, such as all the mRNA molecules in a single cell.

The term “transcript” refers to a length of RNA or DNA that has been transcribed, respectively, from a DNA or RNA template.

As used herein, the term “genomic locus” or “locus” (plural loci) is the specific location of a gene or DNA sequence on a chromosome. A “gene” refers to stretches of DNA or RNA that encode a polypeptide or an RNA chain playing a functional role in an organism and hence is the molecular unit of heredity in living organisms. For the purpose of this invention, it may be considered that genes include regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Variant”, as the term is used herein, is a nucleic acid sequence or a peptide sequence that differs in sequence from a reference nucleic acid sequence or peptide sequence respectively (e.g., the wild type), but retains one or more characteristic properties of the reference nucleic acid sequence. In some instances, changes in the sequence of a nucleic acid leading to a variant may not alter the amino acid sequence of a peptide encoded by the reference nucleic acid or, alternatively, may result in amino acid substitutions, additions, deletions, fusions and/or truncations. A variant of a nucleic acid or peptide can be naturally occurring such as an allelic variant, or can be a variant that is not known to occur naturally (e.g., a synthetic variant). Non-naturally occurring variants of nucleic acids and peptides may be made by, for example, mutagenesis techniques or by direct synthesis.

As used herein, unless otherwise evident from context, the term “integrate” or the concept of integrating means that several pieces of information that may be from the same RNA sequence or from several RNA sequences are combined to arrive at a decision or determination of a fact, condition, or to improve or modify an existing decision or determination. The term “integrate” can be used to refer to providing several pieces of information to an algorithm, including an artificial intelligence agent such as a machine learning model, and using the output to make a prediction/determination or improve accuracy of existing prediction/determination.

By “position”, as used herein, is meant a location in the sequence of a protein or a nucleic acid. Positions may be numbered sequentially, or according to an established format.

The terms “folding free energy”, “free energy”, “folding energy”, as used interchangeably herein, refer to the energy released by folding an unfolded polynucleotide (e.g., RNA or DNA, etc.) molecule, or, conversely, the amount of energy that must be added in order to unfold a folded polynucleotide (e.g., RNA or DNA, etc.). The “minimum free energy (MFE)” of a polynucleotide (e.g., DNA, RNA, etc.) describes the lowest value of free energy observed for the polynucleotide when assessed for various possible secondary structures. The MFE of a RNA molecule may be used to predict RNA or DNA secondary structure and is affected by the number, composition, and arrangement of the RNA or RNA nucleotides. As is generally true, the more negative free energy a structure has, the more likely is its formation since more stored energy is released by formation of the structure.

The term “secondary structure,” or “secondary structure element” or “secondary structure sequence region” as used herein in reference to nucleic acid sequences (e.g., RNA, DNA, etc.), refers to any non-linear conformation of nucleotide or ribonucleotide units. Such non-linear conformations may include base-pairing interactions within a single nucleic acid polymer or between two polymers. Single-stranded RNA typically forms complex and intricate base-pairing interactions due to its increased ability to form hydrogen bonds stemming from the extra hydroxyl group in the ribose sugar. Examples of secondary structures or secondary structure elements include but are not limited to, for example, stem-loops, hairpin structures, bulges, internal loops, multiloops, coils, random coils, helices, partial helices and pseudoknots. In some embodiments, the term “secondary structure” may refer to a SuRE element. The term “SuRE” stands for stem-loop structured RNA element (SuRE).

The term “parameter” herein refers to a numerical value that characterizes a property of a system such as a physical feature whose value or other characteristic has an impact on a relevant condition of a molecule, such as an oligonucleotide, e.g. an RNA molecule. In some cases, the term parameter is used with reference to a variable that affects the output of a mathematical relation or model, which variable may be an independent variable (i.e., an input to the model) or an intermediate variable based on one or more independent variables. Depending on the scope of a model, an output of one model may become an input of another model, thereby becoming a parameter to the other model.

The term “plurality” refers to more than one element.

The term “in vitro” may be understood in the broadest sense that the use is performed out of the biological context, e.g., outside of the body of a subject or with a sample isolated from its biological context. An in vitro use may, for example, be performed in labware such as test tubes, flasks, Petri dishes, and microtiter plates. Other suitable in vitro uses are well known to the person skilled in the art.

The term “in vivo” refers to an event that takes place in a host organism.

The term ‘complementary binding”, “base pair”, “complementary base pair”, as used herein, with respect to nucleic acids indicates the two nucleotides on opposite polynucleotide strands or sequences connected via hydrogen bonds. For example, in the canonical Watson-Crick DNA base pairing, adenine (A) forms a base pair with thymine (T) and guanine (G) forms a base pair with cytosine (C). In RNA base paring, adenine (A) forms a base pair with uracil (U) and guanine (G) forms a base pair with cytosine (C). Accordingly, the term “base pairing” as used herein indicates formation of hydrogen bonds between base pairs on opposite complementary polynucleotide strands or sequences following the Watson-Crick base pairing rule as will be applied by a skilled person to provide duplex polynucleotides. Accordingly, when two polynucleotide strands, sequences or segments are noted to be binding to each other through complementarily binding or complementarily bind to each other, this indicate that a sufficient number of bases pairs forms between the two strands, sequences or segments to form a thermodynamically stable double-stranded duplex, although the duplex can contain mismatches, bulges and/or wobble base pairs as will be understood by a skilled person.

The term “thermodynamic stability”, as used herein, indicates the lowest energy state of a chemical system. Thermodynamic stability can be used in connection with description of two chemical entities (e.g., two molecules or portions thereof) to compare the relative energies of the chemical entities. For example, when a chemical entity is a polynucleotide, thermodynamic stability can be used in absolute terms to indicate a conformation that is at a lowest energy state, or in relative terms to describe conformations of the polynucleotide or portions thereof to identify the prevailing conformation as a result of the prevailing conformation being in a lower energy state. Thermodynamic stability can be detected using methods and techniques identifiable by a skilled person. For example, for polynucleotides thermodynamic stability can be determined based on measurement of melting temperature Tₘ, among other methods, wherein a higher Tₘ can be associated with a more thermodynamically stable chemical entity as will be understood by a skilled person. Contributors to thermodynamic stability can include, but are not limited to, chemical composition, base composition, neighboring chemical composition, and geometry of the chemical entity. This term may also be applied to two or more conformers of the same chemical entity, e.g., an RNA molecule with more than one possible/likely secondary structure.

The terms “coding sequence,” “coding sequence region,” “coding region,”, “CDS”, or “encoding sequence” when referring to nucleic acid sequences may be used interchangeably herein to refer to the portion of a DNA or RNA sequence, for example, that is or may be translated to protein. The terms “reading frame,” “open reading frame,” and “ORF,” may be used interchangeably herein to refer to a nucleotide sequence that begins with an initiation codon (e.g., ATG) and, in some embodiments, ends with a termination codon (e.g., TAA, TAG, or TGA). Open reading frames may contain introns and exons, and as such, all CDSs are ORFs, but not all ORF are CDSs.

The term “codon” or “triplet” refers to a nucleotide sequence of three nucleotides as three adjacent (attached to each other within a gene) deoxyribose nucleic acids or three adjacent ribose nucleic acids (attached to each other within a transcribed RNA) that encode a specific amino acid or a control signal during transcription or translation, respectively. Several codons may represent the same amino acid, in other words “degenerate codons” or “synonymous codons.” “Degeneracy” in reference to the genetic code means that one amino acid can be encoded by several codons. As one example, CAT or CAC (DNA) and CAC or CAU (RNA) encode or represent the amino acid Histidine. In other words, CAT and CAC are “synonymous.” Further, each particular organism may not use the available codons randomly, but may show a certain preference for having or “using” particular codons for the same amino acid, such that each individual genome may use a preferred set of codons.

The term “sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single-stranded DNA). Sequencing can be performed by various systems currently available, such as, without limitation, a sequencing system by Illumina®, Pacific Biosciences (PacBio®), Oxford Nanopore®, or Life Technologies (Ion Torrent®) Alternatively, or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

The term “conformation”, as used herein in relation to a polymer, e.g., polynucleotide, peptide or polypeptide, is used to refer to a spatial arrangement of atoms. The conformation of a polynucleotide or polypeptide can characterize a conformation of a backbone of the polymer. a conformation of each side chain or a combination thereof.

The term “secondary structure,” or “secondary structure element” or “secondary structure sequence region”, as used herein, in reference to nucleic acid sequences (e.g., RNA, DNA, etc.), refers to any non-linear conformation of nucleotide or ribonucleotide units. Such non-linear conformations may include base-pairing interactions within a single nucleic acid polymer or between two polymers. Single-stranded RNA typically forms complex and intricate base-pairing interactions due to its increased ability to form hydrogen bonds stemming from the extra hydroxyl group in the ribose sugar. Examples of secondary structures or secondary structure elements include but are not limited to, for example, stem-loops, hairpin structures, bulges, internal loops, multiloops, coils, random coils, helices, partial helices and pseudoknots. In some embodiments, the term “secondary structure” may refer to a SuRE element. The term “SuRE” stands for stem-loop structured RNA element (SuRE).

The terms “activate,” “stimulate,” “enhance” “increase” and/or “induce” (and like terms) are used interchangeably to generally refer to improving or increasing, either directly or indirectly, a concentration, level, function, activity, or behavior relative to the natural, expected, or average, or relative to a control condition.

As used herein, the term “suppress,” “decrease,” “interfere,” “inhibit” and/or “reduce” (and like terms) generally refers to reducing, either directly or indirectly, a concentration, level, function, activity, or behavior relative to the natural, expected, or average, or relative to a control condition.

The term “algorithm” as used herein is a broad term and is to be given its ordinary and customary meaning to a person of ordinary skill in the art (and is not to be limited to a special or customized meaning), and furthermore refers without limitation to a computational process (for example, programs) involved in transforming information from one state to another, for example, by using computer processing.

The term “bona fide” as used in this context is used to indicate that the identified candidate riboswitch is in fact a legitimate riboswitch, e.g., a functional riboswitch.

As used herein, the term “reporter” or “reporter molecule” refers to a moiety capable of being detected indirectly or directly. Reporters include, without limitation, a chromophore, a fluorophore, a fluorescent protein, a luminescent protein, a receptor, a hapten, an enzyme, and a radioisotope. An exemplary reporter molecule generates (or extinguishes) a signal following a particular event, the observation of which is desired.

As used herein, the term “reporter protein” refers to a class of reporter molecule, a protein that confers to a cell expressing it a property that is detectable or measurable. Reporter proteins can be used as a selectable marker. Non-limiting examples of reporter proteins include fluorescent proteins, luciferase, β-galactosidase, and various proteins that confer antibiotic resistance. An exemplary reporter protein is a fluorescent protein.

As used herein, the term “fluorescent protein” refers to a protein domain that comprises at least one organic compound moiety that emits fluorescent light in response to the appropriate wavelengths. For example, fluorescent proteins may emit red, blue and/or green light. Such proteins are readily commercially available including, but not limited to i) mCherry (Clonetech Laboratories): excitation: 556/20 nm (wavelength/bandwidth); emission: 630/91 nm; ii) sfGFP (Invitrogen): excitation: 470/28 nm; emission: 512/23 nm; iii) TagBFP (Evrogen): excitation 387/11 nm; emission 464/23 nm.

As used herein, the term “reporter gene” refers to a polynucleotide encoding a reporter molecule that can be detected, either directly or indirectly. Exemplary reporter genes encode, among others, enzymes, fluorescent proteins, bioluminescent proteins, receptors, antigenic epitopes, and transporters.

The term “control” as used herein refers to a predetermined value or range, which is employed as a benchmark against which to assess the measured result.

As used herein, “messenger RNA” or “mRNA” are RNA molecules comprising a sequence that encodes a polypeptide or protein. In general, RNA can be transcribed from DNA. In some cases, precursor mRNA containing non-protein coding regions in the sequence can be transcribed from DNA and then processed to remove all or a portion of the non-coding regions (introns) to produce mature mRNA.

As used herein, unless otherwise dictated by context “nucleotide” or “nt” refers to ribonucleotide.

As used herein, the terms “patient” and “subject” are used interchangeably, and may be taken to mean any living organism which may be treated with compounds found using the present disclosure. As such, the terms “patient” and “subject” include, but are not limited to, any non-human mammal, primate and human.

The term “stop codon” can refer to a three-nucleotide contiguous sequence within messenger RNA that signals a termination of translation. Non-limiting examples include in RNA, UAG (amber), UAA (ochre), UGA (umber, also known as opal) and in DNA TAG, TAA or TGA. Unless otherwise noted, the term can also include nonsense mutations within DNA or RNA that introduce a premature stop codon, causing any resulting protein to be abnormally shortened.

A “therapeutically effective amount” of a composition is an amount sufficient to achieve a desired therapeutic effect, and does not require cure or complete remission.

The terms “treat,” “treated,” “treating”, or “treatment” as used herein have the meanings commonly understood in the medical arts, and therefore does not require cure or complete remission, and therefore includes any beneficial or desired clinical results. Treatment includes eliciting a clinically significant response without excessive levels of side effects. Treatment also includes prolonging survival as compared to expected survival if not receiving treatment.

As used herein, the term “mismatch” refers to a single nucleotide in a guide RNA that is unpaired to an opposing single nucleotide in a target RNA within the guide-target RNA scaffold. A mismatch can comprise any two single nucleotides that do not base pair. Where the number of participating nucleotides on the guide RNA side and the target RNA side exceeds 1, the resulting structure is no longer considered a mismatch, but rather, is considered a bulge or an internal loop, depending on the size of the structural feature. In some embodiments, a mismatch is an A/C mismatch. An A/C mismatch can comprise a C in an engineered guide RNA of the present disclosure opposite an A in a target RNA. An A/C mismatch can comprise an A in an engineered guide RNA of the present disclosure opposite a C in a target RNA. A G/G mismatch can comprise a G in an engineered guide RNA of the present disclosure opposite a G in a target RNA. In some embodiments, a mismatch positioned 5′ of the edit site can facilitate base-flipping of the target A to be edited. A mismatch can also help confer sequence specificity. Thus, a mismatch can be a structural feature formed from latent structure provided by an engineered latent guide RNA.

The term percent “identity,” in the context of two or more nucleic acid or polypeptide sequences, refers to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

It should be understood that the present disclosure includes polynucleotide sequences encoding for any sequence disclosed herein. For example, if an amino acid sequence is provided, the present disclosure also encompasses a polynucleotide sequence encoding for said amino acid sequence. It should be understood that further embodiments include thereof that do not alter the desired properties, as described herein.

As used herein, the term “model” refers to a machine learning model or algorithm.

In some embodiments, a model includes an unsupervised learning algorithm. One example of an unsupervised learning algorithm is cluster analysis. In some embodiments, a model includes a supervised machine learning algorithm. Nonlimiting examples of supervised learning algorithms include, but are not limited to, logistic regression, neural networks, support vector machines, Naive Bayes algorithms, nearest neighbor algorithms, random forest algorithms, decision tree algorithms, boosted trees algorithms, multinomial logistic regression algorithms, linear models, linear regression, Gradient Boosting, mixture models, hidden Markov models, Gaussian NB algorithms, linear discriminant analysis, or any combinations thereof. In some embodiments, a model is a multinomial classifier algorithm. In some embodiments, a model is a 2-stage stochastic gradient descent (SGD) model. In some embodiments, a model is a deep neural network (e.g., a deep-and-wide sample-level model).

Neural networks. In some embodiments, the model is a neural network (e.g., a convolutional neural network and/or a residual neural network). Neural network algorithms, also known as artificial neural networks (ANNs), include convolutional and/or residual neural network algorithms (deep learning algorithms). In some embodiments, neural networks are machine learning algorithms that are trained to map an input dataset to an output dataset, where the neural network includes an interconnected group of nodes organized into multiple layers of nodes. For example, in some embodiments, the neural network architecture includes at least an input layer, one or more hidden layers, and an output layer. In some embodiments, the neural network includes any total number of layers, and any number of hidden layers, where the hidden layers function as trainable feature extractors that allow mapping of a set of input data to an output value or set of output values. In some embodiments, a deep learning algorithm is a neural network including a plurality of hidden layers, e.g., two or more hidden layers. In some instances, each layer of the neural network includes a number of nodes (or “neurons”). In some embodiments, a node receives input that comes either directly from the input data or the output of nodes in previous layers, and performs a specific operation, e.g., a summation operation. In some embodiments, a connection from an input to a node is associated with a parameter (e.g., a weight and/or weighting factor). In some embodiments, the node sums up the products of all pairs of inputs, xi, and their associated parameters. In some embodiments, the weighted sum is offset with a bias, b. In some embodiments, the output of a node or neuron is gated using a threshold or activation function, f, which, in some instances, is a linear or non-linear function. In some embodiments, the activation function is, for example, a rectified linear unit (ReLU) activation function, a Leaky ReLU activation function, or other function such as a saturating hyperbolic tangent, identity, binary step, logistic, arcTan, softsign, parametric rectified linear unit, exponential linear unit, softPlus, bent identity, softExponential, Sinusoid, Sine, Gaussian, or sigmoid function, or any combination thereof.

In some implementations, the weighting factors, bias values, and threshold values, or other computational parameters of the neural network, are “taught” or “learned” in a training phase using one or more sets of training data. For example, in some implementations, the parameters are trained using the input data from a training dataset and a gradient descent, for example, back-propagation, method so that the output value(s) that the ANN computes are consistent with the examples included in the training dataset. In some embodiments, the parameters are obtained from a back propagation neural network training process.

Any of a variety of neural networks are suitable for use in accordance with the present disclosure. Examples include, but are not limited to, feed-forward neural networks, radial basis function networks, recurrent neural networks, residual neural networks, convolutional neural networks, residual convolutional neural networks, and the like, or any combination thereof. In some embodiments, the machine learning makes use of a pre-trained and/or transfer-learned ANN or deep learning architecture. In some implementations, convolutional and/or residual neural networks are used, in accordance with the present disclosure.

For instance, a deep neural network model includes an input layer, a plurality of individually parameterized (e.g., weighted) convolutional layers, and an output scorer. The parameters (e.g., weights) of each of the convolutional layers as well as the input layer contribute to the plurality of parameters (e.g., weights) associated with the deep neural network model. In some embodiments, at least 50 parameters, at least 100 parameters, at least 1000 parameters, at least 2000 parameters, at least 5000 parameters, at least 1×10⁴ parameters, at least 1×10⁵ parameters, at least 1×10⁶ parameters, at least 1×10⁷ parameters, or at least 1×10⁸ parameters are associated with the deep neural network model. As such, deep neural network models require a computer to be used because they cannot be mentally solved. In other words, given an input to the model, the model output needs to be determined using a computer rather than mentally in such embodiments. See, for example, Krizhevsky et al., 2012, “Imagenet classification with deep convolutional neural networks,” in Advances in Neural Information Processing Systems 2, Pereira, Burges, Bottou, Weinberger, eds., pp. 1097-1105, Curran Associates, Inc.; Zeiler, 2012 “ADADELTA: an adaptive learning rate method,” CoRR, vol. abs/1212.5701; and Rumelhart et al., 1988, “Neurocomputing: Foundations of research,” ch. Learning Representations by Back-propagating Errors, pp. 696-699, Cambridge, MA, USA: MIT Press, each of which is hereby incorporated by reference in its entirety.

Neural network algorithms, including convolutional neural network algorithms, suitable for use as models are disclosed in, for example, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference. Additional example neural networks suitable for use as models are disclosed in Duda et al., 2001, Pattern Classification, Second Edition, John Wiley & Sons, Inc., New York; and Hastie et al., 2001, The Elements of Statistical Learning, Springer-Verlag, New York, each of which is hereby incorporated by reference in its entirety. Additional example neural networks suitable for use as models are also described in Draghici, 2003, Data Analysis Tools for DNA Microarrays, Chapman & Hall/CRC; and Mount, 2001, Bioinformatics: sequence and genome analysis, Cold Spring

Harbor Laboratory Press, Cold Spring Harbor, New York, each of which is hereby incorporated by reference in its entirety.

Support vector machines. In some embodiments, the model is a support vector machine (SVM). SVM algorithms suitable for use as models are described in, for example, Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5th Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary labeled data with a hyper-plane that is maximally distant from the labeled data. For certain cases in which no linear separation is possible, SVMs work in combination with the technique of “kernels”, which automatically realizes a non-linear mapping to a feature space. The hyper-plane found by the SVM in feature space corresponds, in some instances, to a non-linear decision boundary in the input space. In some embodiments, the plurality of parameters (e.g., weights) associated with the SVM define the hyper-plane. In some embodiments, the hyper-plane is defined by at least 10, at least 20, at least 50, or at least 100 parameters and the SVM model requires a computer to calculate because it cannot be mentally solved.

Naïve Bayes algorithms. In some embodiments, the model is a Naive Bayes algorithm. Naïve Bayes models suitable for use as models are disclosed, for example, in Ng et al., 2002, “On discriminative vs. generative classifiers: A comparison of logistic regression and naive Bayes,” Advances in Neural Information Processing Systems, 14, which is hereby incorporated by reference. A Naive Bayes model is any model in a family of “probabilistic models” based on applying Bayes' theorem with strong (naïve) independence assumptions between the features. In some embodiments, they are coupled with Kernel density estimation. See, for example, Hastie et al., 2001, The elements of statistical learning: data mining, inference, and prediction, eds. Tibshirani and Friedman, Springer, New York, which is hereby incorporated by reference in its entirety.

Nearest neighbor algorithms. In some embodiments, a model is a nearest neighbor algorithm. In some implementations, nearest neighbor models are memory-based and include no model to be fit. For nearest neighbors, given a query point x₀ (a test subject), the k training points x₍ᵣ₎, r, . . . , k (here the training subjects) closest in distance to x₀ are identified and then the point x₀ is classified using the k nearest neighbors. In some embodiments, Euclidean distance in feature space is used to determine distance as d₍ᵢ₎=∥x₍ᵢ₎−x_{(O)}∥. Typically, when the nearest neighbor algorithm is used, the abundance data used to compute the linear discriminant is standardized to have mean zero and variance 1. In some embodiments, the nearest neighbor rule is refined to address issues of unequal class priors, differential misclassification costs, and feature selection. Many of these refinements involve some form of weighted voting for the neighbors. For more information on nearest neighbor analysis, see Duda, Pattern Classification, Second Edition, 2001, John Wiley & Sons, Inc; and Hastie, 2001, The Elements of Statistical Learning, Springer, New York, each of which is hereby incorporated by reference.

A k-nearest neighbor model is a non-parametric machine learning method in which the input consists of the k closest training examples in feature space. In some embodiments, the output is a class membership. An object is classified by a plurality vote of its neighbors, with the object being assigned to the class most common among its k nearest neighbors (k is a positive integer, typically small). If k=1, then the object is simply assigned to the class of that single nearest neighbor. See, Duda et al., 2001, Pattern Classification, Second Edition, John Wiley & Sons, which is hereby incorporated by reference. In some embodiments, the k-nearest neighbor model is used for regression and the output is a prediction of a property value of the object determined as an average of the values of the k nearest neighbors. In some embodiments, the number of distance calculations needed to solve the k-nearest neighbor model is such that a computer is used to solve the model for a given input because it cannot be mentally performed.

Random forest, decision tree, and boosted tree algorithms. In some embodiments, the model is a decision tree. Decision trees suitable for use as models are described generally by Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. For example, one specific algorithm is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York, pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests—Random Features,” Technical Report 567, Statistics Department, U.C. Berkeley, September 1999, which is hereby incorporated by reference in its entirety. In some embodiments, the decision tree model includes at least 10, at least 20, at least 50, or at least 100 parameters (e.g., weights and/or decisions) and requires a computer to calculate because it cannot be mentally solved.

Regression. In some embodiments, the model uses a regression algorithm. In some embodiments, a regression algorithm is any type of regression. For example, in some embodiments, the regression algorithm is logistic regression. In some embodiments, the regression algorithm is logistic regression with lasso, L2 or elastic net regularization. In some embodiments, those extracted features that have a corresponding regression coefficient that fails to satisfy a threshold value are pruned (removed from) consideration. In some embodiments, a generalization of the logistic regression model that handles multicategory responses is used as the model. Logistic regression algorithms are disclosed in Agresti, An Introduction to Categorical Data Analysis, 1996, Chapter 5, pp. 103-144, John Wiley & Son, New York, which is hereby incorporated by reference. In some embodiments, the model makes use of a regression model disclosed in Hastie et al., 2001, The Elements of Statistical Learning, Springer-Verlag, New York. In some embodiments, the logistic regression model includes at least 10, at least 20, at least 50, at least 100, or at least 1000 parameters (e.g., weights) and requires a computer to calculate because it cannot be mentally solved.

Linear discriminant analysis algorithms. In some embodiments, linear discriminant analysis (LDA), normal discriminant analysis (NDA), or discriminant function analysis is a generalization of Fisher's linear discriminant, a method used in statistics, pattern recognition, and machine learning to find a linear combination of features that characterizes or separates two or more classes of objects or events. In some embodiments, the resulting combination is used as the model (linear model) in some embodiments of the present disclosure.

Mixture model and Hidden Markov model. In some embodiments, the model is a mixture model, such as that described in Mclachlan et al., Bioinformatics 18 (3): 413-422, 2002, which is hereby incorporated by reference in its entirety. In some embodiments, in particular, those embodiments including a temporal component, the model is a hidden Markov model such as described by Schliep et al., 2003, Bioinformatics 19 (1): 1255-i263, which is hereby incorporated by reference in its entirety.

Clustering. In some embodiments, the model is an unsupervised clustering model. In some embodiments, the model is a supervised clustering model. Clustering algorithms suitable for use as models are described, for example, at pages 211-256 of Duda and Hart, Pattern Classification and Scene Analysis, 1973, John Wiley & Sons, Inc., New York, (hereinafter “Duda 1973”) which is hereby incorporated by reference in its entirety. As an illustrative example, in some embodiments, the clustering problem is described as one of finding natural groupings in a dataset. To identify natural groupings, two issues are addressed. First, a way to measure similarity (or dissimilarity) between two samples is determined. This metric (e.g., similarity measure) is used to ensure that the samples in one cluster are more like one another than they are to samples in other clusters. Second, a mechanism for partitioning the data into clusters using the similarity measure is determined. One way to begin a clustering investigation is to define a distance function and to compute the matrix of distances between all pairs of samples in the training set. If distance is a good measure of similarity, then the distance between reference entities in the same cluster is significantly less than the distance between the reference entities in different clusters. However, in some implementations, clustering does not use a distance metric. For example, in some embodiments, a nonmetric similarity function s(x, x′) is used to compare two vectors x and x′. In some such embodiments, s(x, x′) is a symmetric function whose value is large when x and x′ are somehow “similar.” Once a method for measuring “similarity” or “dissimilarity” between points in a dataset has been selected, clustering uses a criterion function that measures the clustering quality of any partition of the data. Partitions of the dataset that extremize the criterion function are used to cluster the data. Particular exemplary clustering techniques contemplated for use in the present disclosure include, but are not limited to, hierarchical clustering (agglomerative clustering using a nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering. In some embodiments, the clustering includes unsupervised clustering (e.g., with no preconceived number of clusters and/or no predetermination of cluster assignments).

Ensembles of models and boosting. In some embodiments, an ensemble (two or more) of models is used. In some embodiments, a boosting technique such as AdaBoost is used in conjunction with many other types of learning algorithms to improve the performance of the model. In this approach, the output of any of the models disclosed herein, or their equivalents, is combined into a weighted sum that represents the final output of the boosted model. In some embodiments, the plurality of outputs from the models is combined using any measure of central tendency known in the art, including but not limited to a mean, median, mode, a weighted mean, weighted median, weighted mode, etc. In some embodiments, the plurality of outputs is combined using a voting method. In some embodiments, a respective model in the ensemble of models is weighted or unweighted.

In some embodiments, the model is a reinforcement learning model. In some embodiments, the reinforcement learning model comprises four main elements—an agent, a policy, a reward signal, and a value function, where the behavior of the agent is defined in terms of the policy. In some embodiments, the reinforcement learning model comprises a learning algorithm. In some implementations, the learning algorithm is an on-policy learning algorithm or an off-policy learning algorithms. On-Policy learning algorithms evaluate and improve the same policy which is being used to select the agent's actions. Off-Policy learning algorithms evaluate and improve policies that are different from the policy being used for action selection. Reinforcement learning is further described, for example, in Sutton R S, Barto A G, “Reinforcement learning: an introduction,” IEEE Transactions on Neural Networks. 1998; 9 (5): 1054-1054, which is hereby incorporated herein by reference in its entirety. In some embodiments, the reinforcement learning model includes at least 10, at least 100, at least 1000, at least 10,000, at least 100,000, at least 1×10⁶, at least 1×10⁷, or more parameters. In some embodiments, the reinforcement learning model includes no more than 1×10⁸, no more than 1×10⁷, no more than 1×10⁶, no more than 100,000, no more than 10,000, no more than 1000, or no more than 100 parameters. In some embodiments, the reinforcement learning model consists of from 10 to 1000, from 100 to 100,000, from 10,000 to 1×10⁷, or from 1×10⁶ to 1×10⁸ parameters. In some embodiments, the plurality of parameters for the reinforcement learning model falls within another range starting no lower than 10 parameters and ending no higher than 1×10⁸ parameters.

As used herein, the term “parameter” refers to any coefficient or, similarly, any value of an internal or external element (e.g., a weight and/or a hyperparameter) in an algorithm, model, regressor, and/or classifier that can affect (e.g., modify, tailor, and/or adjust) one or more inputs, outputs, and/or functions in the algorithm, model, regressor and/or classifier. For example, in some embodiments, a parameter refers to any coefficient, weight, and/or hyperparameter that can be used to control, modify, tailor, and/or adjust the behavior, learning, and/or performance of an algorithm, model, regressor, and/or classifier. In some instances, a parameter is used to increase or decrease the influence of an input (e.g., a feature) to an algorithm, model, regressor, and/or classifier. As a nonlimiting example, in some embodiments, a parameter is used to increase or decrease the influence of a node (e.g., of a neural network), where the node includes one or more activation functions. Assignment of parameters to specific inputs, outputs, and/or functions is not limited to any one paradigm for a given algorithm, model, regressor, and/or classifier but can be used in any suitable algorithm, model, regressor, and/or classifier architecture for a desired performance. In some embodiments, a parameter has a fixed value. In some embodiments, a value of a parameter is manually and/or automatically adjustable. In some embodiments, a value of a parameter is modified by a validation and/or training process for an algorithm, model, regressor, and/or classifier (e.g., by error minimization and/or backpropagation methods). In some embodiments, an algorithm, model, regressor, and/or classifier of the present disclosure includes a plurality of parameters. In some embodiments, the plurality of parameters is n parameters, where: n≥2; n≥5; n≥10; n≥25; n≥40; n≥50; n≥75; n≥100; n≥125; n≥150; n≥200; n≥225; n≥250; n≥350; n≥500; n≥600; n≥750; n≥1,000; n≥2,000; n≥4,000; n≥5,000; n≥7,500; n≥10,000; n≥20,000; n≥40,000; n≥75,000; n≥100,000; n≥200,000; n≥500,000, n≥1×10⁶, n≥5×10⁶, n≥1×10⁷, n≥1×10⁸, or ≥1×10⁹. In some embodiments, the plurality of parameters comprises no more than 1×10¹⁰, no more than 1×10⁹, no more than 1×10⁸, no more than 1×10⁷, no more than 1×10⁶, no more than 1×10⁵, no more than 1×10⁴, or no more than 1×10³. As such, the algorithms, models, regressors, and/or classifiers of the present disclosure cannot be mentally performed. In some embodiments n is between 10,000 and 1×10⁷, between 100,000 and 5×10⁶, or between 500,000 and 1×10⁶. In some embodiments, the plurality of parameters falls within another range starting no lower than 2 parameters and ending no higher than 1×10¹⁰ parameters. In some embodiments, the algorithms, models, regressors, and/or classifier of the present disclosure operate in a k-dimensional space, where k is a positive integer of 5 or greater (e.g., 5, 6, 7, 8, 9, 10, etc.). As such, the algorithms, models, regressors, and/or classifiers of the present disclosure cannot be mentally performed.

As used herein, the term “untrained model” refers to a machine learning model or algorithm, such as a classifier or a neural network, that has not been trained on a target dataset. In some embodiments, “training a model” (e.g., “training a neural network”) refers to the process of training an untrained or partially trained model (e.g., “an untrained or partially trained neural network”). Moreover, it will be appreciated that the term “untrained model” does not exclude the possibility that transfer learning techniques are used in such training of the untrained or partially trained model. For instance, Fernandes et al., 2017, “Transfer Learning with Partial Observability Applied to Cervical Cancer Screening,” Pattern Recognition and Image Analysis: 8^{th} Iberian Conference Proceedings, 243-250, which is hereby incorporated by reference, provides non-limiting examples of such transfer learning. In instances where transfer learning is used, the untrained model described above is provided with additional data over and beyond that of the primary training dataset. Typically, this additional data is in the form of parameters (e.g., coefficients, weights, and/or hyperparameters) that were learned from another, auxiliary training dataset. Moreover, while a description of a single auxiliary training dataset has been disclosed, it will be appreciated that there is no limit on the number of auxiliary training datasets that can be used to complement the primary training dataset in training the untrained model in the present disclosure. For instance, in some embodiments, two or more auxiliary training datasets, three or more auxiliary training datasets, four or more auxiliary training datasets or five or more auxiliary training datasets are used to complement the primary training dataset through transfer learning, where each such auxiliary dataset is different than the primary training dataset. Any manner of transfer learning is used, in some such embodiments. For instance, consider the case where there is a first auxiliary training dataset and a second auxiliary training dataset in addition to the primary training dataset. In such a case, the parameters learned from the first auxiliary training dataset (by application of a first model to the first auxiliary training dataset) are applied to the second auxiliary training dataset using transfer learning techniques (e.g., a second model that is the same or different from the first model), which in turn results in a trained intermediate model whose parameters are then applied to the primary training dataset and this, in conjunction with the primary training dataset itself, is applied to the untrained model. Alternatively, in another example embodiment, a first set of parameters learned from the first auxiliary training dataset (by application of a first model to the first auxiliary training dataset) and a second set of parameters learned from the second auxiliary training dataset (by application of a second model that is the same or different from the first model to the second auxiliary training dataset) are each individually applied to a separate instance of the primary training dataset (e.g., by separate independent matrix multiplications) and both such applications of the parameters to separate instances of the primary training dataset in conjunction with the primary training dataset itself (or some reduced form of the primary training dataset such as principal components or regression coefficients learned from the primary training set) are then applied to the untrained model in order to train the untrained model.

In some embodiments, the methods described herein include inputting information into a model comprising a plurality of parameters, where the model applies the plurality parameters to the information through a plurality of instructions to generate an output from the model.

In some embodiments, the model comprises a language model, a transformer model, a large language model (LLM), an encoder, a decoder, an encoder-decoder hybrid model, a generative pre-trained transformer (GPT) model, a Bidirectional Encoder Representations from Transformers (BERT) model, or a multiple sequence alignment (MSA) transformer model.

In some embodiments, the attention mechanism comprises one selected from the group consisting of dot product attention, query-key-value attention, Luong attention, and Bahdanau attention.

In some embodiments, the attention mechanism is applied directly to all or a portion of the data structure input into the model. In some embodiments, the attention mechanism is applied to an embedding of all or a portion of the data structure input into the model. In some embodiments, an attention mechanism is a mapping of a query (e.g., the data structure or embedding thereof) and a set of key-value pairs to an output where the query, keys, values, and output are all vectors. In some such embodiments, the output is computed as a weighted sum of the values, where the weight assigned to each value is computed by a compatibility function of the query with the corresponding key. Example attention mechanisms are described in Chaudhari et al., Jul. 12, 2021 “An Attentive Survey of Attention Models,” arXiv: 1904-02874v3, and Vaswani et al., “Attention is All You Need,” 31st Conference on Neural Information Processing Systems (NIPS 2017), Long Beach, California, USA, each of which is hereby incorporated by reference.

Advantageously, transformer-based models can handle inputs with variable lengths, making such models generalizable to different micro-footprints or macro-footprints for in-cell experiments. Additionally, transformers are less prone to overfitting, allowing for easy integration of historical or future datasets to further enhance the model performance.

As used herein, the term “instruction” refers to an order given to a computer processor by a computer program. On a digital computer, in some embodiments, each instruction is a sequence of 0s and 1s that describes a physical operation the computer is to perform. Such instructions can include data transfer instructions and data manipulation instructions. In some embodiments, each instruction is a type of instruction in an instruction set that is recognized by a particular processor type used to carry out the instructions. Examples of instruction sets include, but are not limited to, Reduced Instruction Set Computer (RISC), Complex Instruction Set Computer (CISC), Minimal instruction set computers (MISC), Very long instruction word (VLIW), Explicitly parallel instruction computing (EPIC), and One instruction set computer (OISC).

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, and components have not been described in detail so as not to unnecessarily obscure aspects of the embodiments.

It will also be understood that, although the terms first, second, etc. may be used herein to describe various elements, these elements should not be limited by these terms. These terms are only used to distinguish one element from another. For instance, a first subject could be termed a second subject, and, similarly, a second subject could be termed a first subject, without departing from the scope of the present disclosure. The first subject and the second subject are both subjects, but they are not the same subject.

The terminology used in the present disclosure is for the purpose of describing particular embodiments only and is not intended to be limiting of the invention. As used in the description of the invention and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The present description includes example systems, methods, techniques, instruction sequences, and computing machine program products that embody illustrative implementations. For purposes of explanation, numerous specific details are set forth in order to provide an understanding of various implementations of the inventive subject matter. It will be evident, however, to those skilled in the art that implementations of the inventive subject matter may be practiced without these specific details. In general, well-known instruction instances, protocols, structures, and techniques have not been shown in detail.

The present description, for purpose of explanation, is described with reference to specific implementations. However, the illustrative discussions below are not intended to be exhaustive or to limit the implementations to the precise forms disclosed. Many modifications and variations are possible in view of the above teachings. The implementations are chosen and described in order to best explain the principles and their practical applications, to thereby enable others skilled in the art to best utilize the implementations and various implementations with various modifications as are suited to the particular use contemplated.

In the interest of clarity, not all of the routine features of the implementations described herein are shown and described. It will be appreciated that, in the development of any such actual implementation, numerous implementation-specific decisions are made in order to achieve the designer's specific goals, such as compliance with use case- and business-related constraints, and that these specific goals will vary from one implementation to another and from one designer to another. Moreover, it will be appreciated that such a design effort might be complex and time-consuming, but nevertheless be a routine undertaking of engineering for those of ordering skill in the art having the benefit of the present disclosure.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

As used herein, the term “about” or “approximately” can mean within an acceptable error range for the particular value as determined by one of ordinary skill in the art, which can depend in part on how the value is measured or determined, e.g., the limitations of the measurement system. For example, “about” can mean within 1 or more than 1 standard deviation, per the practice in the art. “About” can mean a range of ±20%, ±10%, ±5%, or ±1% of a given value. Where particular values are described in the application and claims, unless otherwise stated, the term “about” means within an acceptable error range for the particular value. The term “about” can have the meaning as commonly understood by one of ordinary skill in the art. The term “about” can refer to ±10%. The term “about” can refer to ±5%.

### 8.3. Example System Embodiments

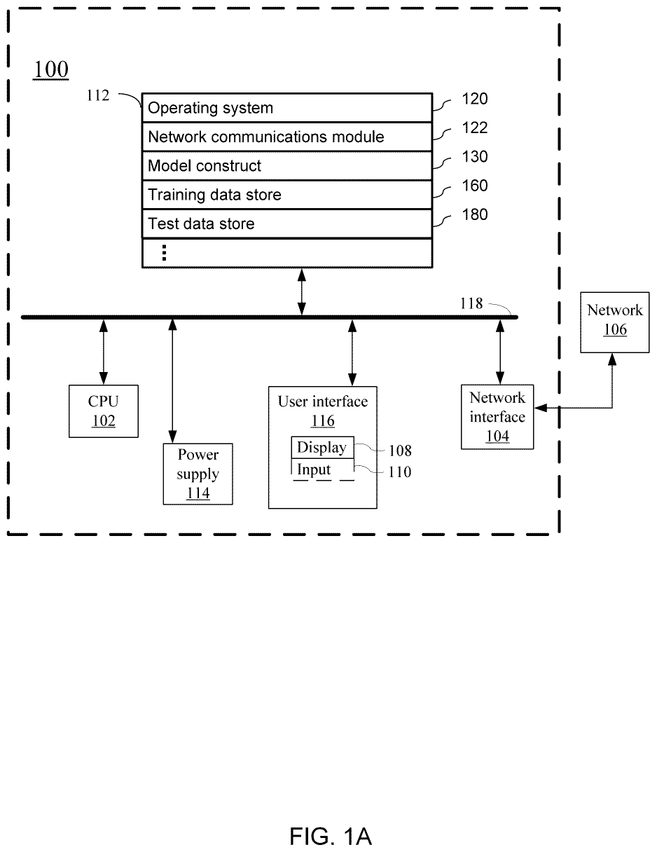

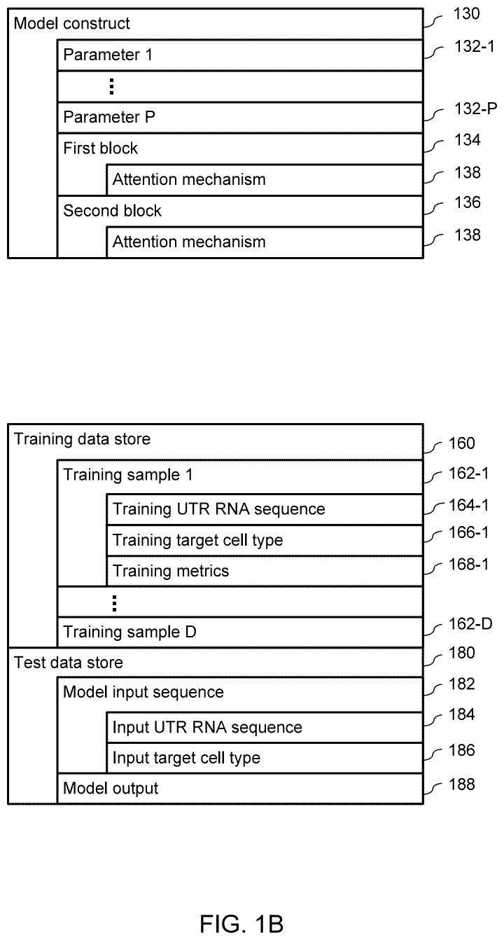

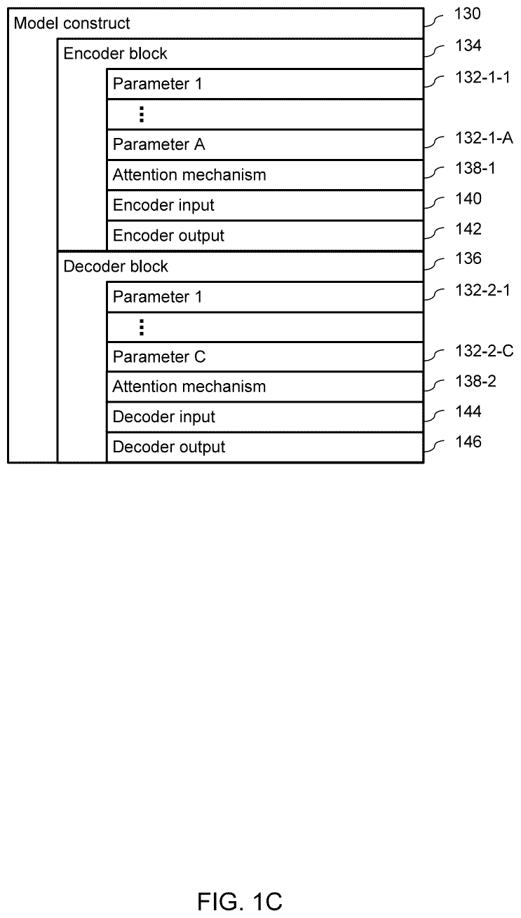

In the present disclosure, unless expressly stated otherwise, descriptions of devices and systems will include implementations of one or more computers. For instance, and for purposes of illustration in FIGS. 1A-D, a computer system 100 is represented as single device that includes all the functionality of the computer system 100. However, the present disclosure is not limited thereto. For instance, in some embodiments, the functionality of the computer system 100 is spread across any number of networked computers and/or reside on each of several networked computers and/or by hosted on one or more virtual machines and/or containers at a remote location accessible across a communications network (e.g., communications network 106). One of skill in the art will appreciate that a wide array of different computer topologies is possible for the computer system 100, and other devices and systems of the preset disclosure, and that all such topologies are within the scope of the present disclosure. Moreover, rather than relying on a physical communications network 106, the illustrated devices and systems may wirelessly transmit information between each other. As such, the exemplary topology shown in FIGS. 1A-C merely serves to describe the features of an embodiment of the present disclosure in a manner that will be readily understood to one of skill in the art.

FIG. 1A depicts a block diagram of a computer system 100 according to some embodiments of the present disclosure. The computer system 100 at least facilitates predicting cell-type-specific activity of an untranslated region (UTR) RNA sequence.

In some embodiments, the prediction of cell-type-specific activity of an untranslated region (UTR) RNA sequence and/or the generation of UTR RNA with predefined cell-type-specific activity is prepared at the computer system 100. In some embodiments, UTR RNAis then provided (e.g., communicated through communication network 106) to a subject through a display of a respective client device. However, the present disclosure is not limited thereto.

In some embodiments, the communication network 106 optionally includes the Internet, one or more local area networks (LANs), one or more wide area networks (WANs), other types of networks, or a combination of such networks.

Examples of communication networks 106 include the World Wide Web (WWW), an intranet and/or a wireless network, such as a cellular telephone network, a wireless local area network (LAN) and/or a metropolitan area network (MAN), and other devices by wireless communication. The wireless communication optionally uses any of a plurality of communications standards, protocols and technologies, including Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11ac, IEEE 802.11ax, IEEE 802.11b, IEEE 802.11g and/or IEEE 802.11n), voice over Internet Protocol (VOIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of this document.

In various embodiments, the computer system 100 includes one or more processing units (CPUs, processing cores, etc.) 102, a network or other communications interface 104, and memory 112. In some embodiments, the computer system 100 includes a power supply 114 configured to provide a current to one or more components and/or hardware devices of the computer system 100 or a remote device.

In some embodiments, the computer system 100 includes a user interface 116. The user interface 116 typically includes a display 108 for presenting media, such as an output from a model of the present disclosure. In some embodiments, the display 108 is integrated within the computer system (e.g., housed in the same chassis as the CPU 102 and memory 112). In some embodiments, the computer system 100 includes one or more input device(s) 110, which allow a subject to interact with the computer system 100. In some embodiments, the one or more input devices 110 include a keyboard, a mouse, and/or other input mechanisms. Alternatively, or in addition, in some embodiments, the display 108 includes a touch-sensitive surface (e.g., where display 108 is a touch-sensitive display or computer system 100 includes a touch pad).

In some embodiments, the computer system 100 presents media to a user through the display 108. Examples of media presented by the display 108 include a prediction of a deamination efficiency or specificity, a generation of the candidate sequence for the UTR RNA, an output from a model, or a combination thereof. In typical embodiments, the media is presented by the display 108 through a client application.

In some embodiments, memory 112 includes high-speed random access memory, such as DRAM, SRAM, DDR RAM, or other random access solid state memory devices, and optionally also includes non-volatile memory, such as one or more magnetic disk storage devices, optical disk storage devices, flash memory devices, or other non-volatile solid state storage devices. In some embodiments, memory 112 optionally includes one or more storage devices remotely located from the CPU(s) 102. In some embodiments, memory 112, or alternatively the non-volatile memory device(s) within memory 112, includes a non-transitory computer readable storage medium. Access to memory 112 by other components of the computer system 100, such as the CPU(s) 102, is, optionally, controlled by a controller. In some embodiments, the memory 112 include mass storage that is remotely located with respect to the CPU(s) 102. In other words, some data stored in memory 112 is in fact hosted on devices that are external to the computer system 100, but that can be electronically accessed by the computer system 100 over an Internet, intranet, or other form of network 106 or electronic cable using communication interface 104.

In some embodiments, the memory 112 of the computer system 100 for predicting cell-type-specific activity of an untranslated region (UTR) RNA sequence stores:
an optional operating system 120 (e.g., ANDROID, IOS, DARWIN, RTXC, LINUX, UNIX, OS X, WINDOWS, or an embedded operating system such as VxWorks) that includes procedures for handling various basic system services;
an optional network communications module 122 associated with the computer system 100 that identifies the computer system 100 (e.g., within the communication network 106);
an optional model construct 130;
an optional training data store 160; and
an optional test data store 180.

In some embodiments, the computer system 100 includes an operating system 120 that includes procedures for handling various basic system services. The operating system 120 (e.g., iOS, DARWIN, RTXC, LINUX, UNIX, OS X, WINDOWS, or an embedded operating system such as VxWorks) includes various software components and/or drivers for controlling and managing general system tasks (e.g., memory management, storage device control, power management, etc.) and facilitates communication between various hardware and software components of the computer system.

In some embodiments, an optional network communications module 122 is associated with the computer system 100. The optional network communications module 122 is utilized to, at least, uniquely identify the computer system 100 from other devices and components (e.g., uniquely identify computer system 100 from a first client device, etc.). For instance, in some embodiments, the optional network communications module is utilized to receive information from a client device.

Referring to FIG. 1B, in some embodiments, the system 100 at least includes instructions for predicting cell-type-specific activity of an UTR RNA sequence, and/or optimizing a model to predict the same, and optionally repeating execution of the instructions for a plurality of UTR RNA sequences, in accordance with some embodiments of the present disclosure.

In some embodiments, the optional model construct 130 comprises a plurality of parameters 132 (e.g., 132-1, . . . 132-P) across a first block 134 and a second block 136, where each of the first block 134 and the second block 136 comprises an attention mechanism 138, and the plurality of parameters reflects, at least in part, pretraining information for a plurality of pretraining samples comprising, for each respective pretraining sample in the plurality of pretraining samples, a corresponding UTR RNA sequence and a respective target cell type. In some embodiments, the optional test data store 180 comprises, as input 182 to the model, first test information comprising a respective UTR RNA sequence and a respective target cell type and, as output 184 from the model, an indication of cell-type-specific activity associated with the UTR RNA sequence and target cell type 182, or a representation thereof. In some embodiments, the optional training data store 160 comprises a plurality of training samples 162 (e.g., 162-1, . . . 162-D), where each respective training sample in the plurality of training samples comprises training information comprising (i) a corresponding training UTR RNA sequence 164 (e.g., 164-1) and a respective target cell type 166 (e.g., 166-1), and (ii) a corresponding training set of one or more metrics 168 (e.g., 168-1) for the cell-type-specific activity and/or the delta activity for each target cell type in the plurality of target cell types of the UTR RNA sequence; thereby updating the plurality of parameters 132.

Referring to FIG. 1C, alternatively or additionally, in some embodiments, the optional model construct 130 comprises an encoder block 134, and a decoder block 136.

In some embodiments, the encoder block 134 comprises a first set of parameters 132-1 (e.g., 132-1-1, . . . 132-1-A), in a plurality of parameters of the model. In some embodiments, the encoder block 134 comprises an attention mechanism 138 (e.g., 138-1). In some embodiments, the encoder block 134-1 (i) receives, as input 140, a respective UTR RNA sequence and a corresponding target cell type 182, or a representation thereof, and (ii) generates, as output 142-1, a representation of UTR RNA sequence and target cell type.

In some embodiments, the decoder block 136 comprises a second set of parameters 132-2 (e.g., 132-2-1, . . . 132-2-C), in the plurality of parameters of the model. In some embodiments, the decoder block 136 comprises an attention mechanism 138 (e.g., 138-2) that receives, as input 144, the output from the encoder block 142. In some embodiments, the decoder block includes an output 144 that comprises, as output 188 from the model 130, a predicted set of one or more metrics for the cell-type-specific activity and/or the delta activity for each target cell type in the plurality of target cell types.

In some respects, the present disclosure harnesses the power of machine learning to evaluate, predict, determine, and/or generate UTR RNA sequences, for example for use in regulating expression of a target gene in a target cell. In some implementations, the generated UTR RNA sequences can be or are used to treat, ameliorate, or fix a disease or a condition associated with expression of the target gene in a subject. In some embodiments, a generated UTR RNA sequence obtained as disclosed herein can be or is administered to a subject for use in gene therapy. For instance, as described above, delivery of the UTR RNA sequence allows for programmable and precise RNA therapy.

In some embodiments, methods disclosed herein are performed at a computer system comprising at least one processor and a memory storing at least one program for execution by the at least one processor. In some embodiments, the method further includes obtaining a model 130 comprising a plurality of parameters 132 across a first block 134 and a second block 136. In some embodiments, each of the first block 134 and the second block 136 comprises an attention mechanism 138.

In some embodiments, the model comprises a LegNet architecture. In some embodiments, the model further comprises a cold diffusion model, a genetic algorithm, a random sampling model, or a combination thereof.

In some embodiments, the attention mechanism comprises one selected from the non-limiting group consisting of dot product attention, query-key-value attention, Luong attention, and Bahdanau attention. Example attention mechanisms are described in Chaudhari et al., Jul. 12, 2021 “An Attentive Survey of Attention Models,” arXiv: 1904-02874v3, and Vaswani et al., “Attention is All You Need,” 31st Conference on Neural Information Processing Systems (NIPS 2017), Long Beach, California, USA, each of which is hereby incorporated by reference. The attention mechanism draws upon the inference that some portions of UTR RNA sequence, secondary structure, tertiary structure, or any combinations thereof, are more important than others and thus some portions (elements or sets of elements) within the data structure (or embedding thereof) are more important than other portions. The attention mechanism is trained to discover such importance using training UTR RNA and then apply this learned (trained) observation against the data structure (or embedding thereof) for the UTR RNA to form the attention embedding. Thus, the attention mechanism incorporates this notion of relevance by allowing the portion of the model downstream of the attention mechanism to dynamically pay attention to only certain parts of the input data, that help in performing the task at hand (e.g., predicting activity of a UTR RNA) effectively.

In some embodiments, the first block is an encoder block and the second block is a decoder block. In some embodiments, the model comprises one or more encoder blocks and one or more decoder blocks. In some embodiments, the model comprises two or more encoder blocks and a decoder block. In some embodiments, the model consists of two encoder blocks and a decoder block.

In some embodiments, the model comprises an encoder block and a decoder block; where the encoder block is configured to process the representation of each respective UTR RNA sequence in the plurality of UTR RNA sequences through a sequence of computational operations, each computational operation comprising: (i) a convolutional operation, (ii) an activation function, (iii) a normalization operation, (iv) a feature recalibration operation, and (v) a residual connection that combines outputs with its input; and generate one or more embeddings representing the representation of each respective UTR RNA sequence in the plurality of UTR RNA sequences. The decoder block is configured to process the one or more embeddings generated by the encoder block through additional computational operations to refine representations of each respective UTR RNA sequence in the plurality of UTR RNA sequences; and produce for each respective UTR RNA sequence in the plurality of UTR RNA sequences, a predicted set of metrics for the cell-type-specific activity and/or the delta activity for each target cell type in a plurality of target cell types.

In some embodiments, the model comprises an encoder block and a decoder block, where the encoder block is configured to process the representation of each respective UTR RNA sequence in the plurality of UTR RNA sequences through a sequence of computational operations organized hierarchically. The encoder block begins with a stem block comprising a convolutional operation with a kernel size of 3 and 128 output channels to extract basic sequence features, followed by batch normalization and a Swish Linear Unit (SLU) activation function to introduce non-linearity. The processed representation is then passed through multiple EfficientNet-like convolutional blocks, each comprising depthwise separable convolutions for efficient feature extraction, batch normalization, SLU activation, residual connections to preserve feature information, and a feature recalibration operation implemented as a squeeze-and-excitation (SE) block to adaptively recalibrate feature importance across channels. The encoder block outputs one or more embeddings representing the sequence-specific features of each respective UTR RNA sequence, which are provided as input to the decoder block. The decoder block is configured to process the embeddings through additional convolutional layers with residual connections, batch normalization, and SLU activation to refine the feature representations further. The refined embeddings are passed through a per-channel concatenation operation to enhance feature specificity and subsequently processed by a head block comprising a 1×1 convolutional layer with 256 output channels, batch normalization, and activation to reduce dimensionality. The decoder block produces, for each respective UTR RNA sequence, a predicted set of metrics, such as those corresponding to a cell-type-specific activity value for each respective target cell type in a plurality of target cell types and/or a delta activity value. In some embodiments, the output from the decoder block is iteratively fed back to the encoder block to refine the generated UTR RNA sequences and improve prediction accuracy in subsequent iterations.

### 8.4. Additional Exemplary Embodiments

Embodiment 1. A method for predicting cell-type-specific activity of an untranslated region (UTR) RNA sequence, comprising:
A) obtaining UTR RNA sequence data corresponding to a plurality of UTR RNA sequences;
B) processing the UTR RNA sequence data using a model, wherein the model is configured to, for each respective UTR RNA sequence in the plurality of UTR RNA sequences:
   predict a cell-type-specific activity of the respective UTR RNA sequence for each target cell type in a plurality of target cell types, and/or
   predict a delta activity of the UTR RNA sequence for each target cell type in the plurality of target cell types, wherein the delta activity is defined as difference between the predicted cell-type-specific activity specific to the respective target cell type and an average activity across the plurality of target cell types; and
C) outputting, for each respective UTR RNA sequence in the plurality of UTR RNA sequences, a predicted set of metrics for the cell-type-specific activity and/or the delta activity for each target cell type in the plurality of target cell types.

Embodiment 2. The method of embodiment 1, wherein the cell-type-specific activity of an UTR RNA sequence quantifies effect of the UTR RNA sequence on translation and/or mRNA stability of a target gene under suitable conditions, when the UTR RNA sequence and the target gene are operably linked in an expression construct.

Embodiment 3. The method of embodiment 1, further comprising validating the outputted sets of metrics with one or more in vitro or in vivo assays, and feeding validation data obtained from the one or more in vitro or in vivo assays back to the model, thereby improving accuracy of prediction by the model

Embodiment 4. The method of embodiment 3, comprising validating the outputted sets of metrics with a Massively Parallel Reporter Assay (MPRA).

Embodiment 5. The method of embodiment 1, wherein the model is trained with a Massively Parallel Reporter Assay (MPRA) dataset to predict cell-type-specific activity and delta activity of RNA sequences corresponding to one or more target cell types, wherein the MPRA dataset comprises:
the plurality of UTR RNA sequences, and
for each respective UTR RNA in the plurality of UTR RNA sequences, measurements of corresponding cell-type-specific activity specific to the one or more target cell types, measured from the MPRA.

Embodiment 6. The method of embodiment 5, wherein the plurality of target cell types comprise cells from one or more tissues selected from the group consisting of blood tissue, colon tissue, ovarian tissue, breast tissue, and liver tissue.

Embodiment 7. The method of embodiment 1, wherein the plurality of UTR RNA sequences in the MPRA dataset comprises at least 1,000, at least 10,000, at least 100,000, or at least 1×106 5′ UTR RNA sequences; or at least 1,000, at least 10,000, at least 100,000, or at least 1×106 3′ UTR RNA sequences.

Embodiment 8. The method of embodiment 1, wherein each UTR RNA sequence in the plurality of the UTR RNA sequences comprises at most 50, at most 100, at most 150, at most 200, at most 250, at most 300, at most 350, at most 400, at most 450, or at most 500 nucleotides.

Embodiment 9. The method of embodiment 1, wherein step A) further comprises obtaining for each respective UTR RNA sequence in the plurality of UTR RNA sequences, additional sequence information comprising triplet phase information for each respective UTR RNA sequence.

Embodiment 10. The method of embodiment 1, wherein, specific to each target cell type in the plurality of target cell types, the outputted set of metrics comprises:
a cell type specificity index (τ), for each respective UTR RNA sequence in the plurality of UTR RNA sequences, wherein τ:
   ranges from 0 to 1,
   indicates ubiquitous activity at 0, and
   indicates exclusive activity in a single cell type at 1; and
a delta activity value (Δ) quantifying difference between the predicted cell-type-specific activity and an average activity across the plurality of target cell types.

Embodiment 11. The method of embodiment 1, wherein the model comprises a LegNet model.

Embodiment 12. The method of embodiment 1, further comprising regulating expression of a target gene in a target cell, comprising:
operably linking an UTR RNA sequence to the target gene in an expression construct, wherein the UTR RNA sequence exhibits cell-type-specific activity when the expression construct is transferred to a corresponding target cell type and under suitable conditions; and
transferring the expression construct to the target cell.

Embodiment 13. A method for predicting regulatory motifs in an UTR RNA sequence, comprising:
A) obtaining UTR RNA sequence data for each respective UTR RNA sequence in a plurality of UTR RNA sequences, and associated activity for each respective UTR RNA sequence;
B) processing the UTR RNA sequence data using a model, wherein the model is configured to, for each respective UTR RNA sequence in the plurality of UTR RNA sequences, identify one or more candidate motifs in each respective UTR RNA sequence;
C) determining, for each respective UTR RNA sequence in the plurality of UTR RNA sequences, correlation of each candidate motif identified sequences with the associated UTR RNA activity; and
D) outputting a ranked list of candidate motifs identified in the plurality of UTR RNA sequences, wherein the ranking is based on correlation between each respective candidate motif and UTR activity.

Embodiment 14. A method for generating a plurality of UTR RNA sequences with predefined cell-type-specific activity, comprising:
A) obtaining input data comprising one or more predefined activity and one or more cell type-specificity constraints;
B) processing the input data using a first portion of a model to generate a plurality of UTR RNA sequences, wherein the first portion of the model is configured to refine the UTR RNA sequences based on the one or more predefined activity and the one or more cell type-specificity constraints;
C) responsive to step B), using a second portion of the model to predict, for each respective UTR RNA sequence in the plurality of UTR RNA sequences generated in step B):
   a cell-type-specific activity of the respective UTR RNA sequence for each target cell type in a plurality of target cell types, and/or
   a delta activity of the respective UTR RNA sequence for each target cell type in the plurality of target cell types, wherein the delta activity is defined as difference between the predicted cell-type-specific activity specific to the respective target cell type and an average activity across the plurality of target cell types;
D) generating, for each respective UTR RNA sequence in the plurality of UTR RNA sequences generated in step B), a predicted set of metrics for the cell-type-specific activity and/or the delta activity for each target cell type in the plurality of target cell types;
E) selecting a sub-plurality of UTR RNA sequences from the plurality of UTR RNA sequences generated in step B), wherein the corresponding predicted sets of metrics for the sub-plurality of UTR RNA sequences satisfy the one or more predefined activity and the one or more cell type-specificity constraints; and
F) outputting the selected sub-plurality of UTR RNA sequences.

Embodiment 15. The method of embodiment 14, wherein the method further comprises:
evaluating each respective UTR RNA sequence in the plurality of UTR RNA sequences generated to calculate a cell type activity difference (CTAD) score, wherein the CTAD score quantifies the difference in predicted activity of a UTR RNA sequence between two target cell types, and
selecting UTR RNA sequences that maximize the CTAD score while satisfying the one or more predefined activity and the one or more cell type-specificity constraints.

Embodiment 16. The method of embodiment 14, wherein the model comprises a cold diffusion model, a genetic algorithm, a random sampling model, or a combination thereof.

Embodiment 17. An UTR RNA sequence having cell-type-specific activity, wherein the UTR RNA sequence is obtained according to the method of embodiment 1.

Embodiment 18. The UTR RNA sequence of embodiment 17, wherein the UTR RNA sequence has cell-type-specific activity in blood tissue, colon tissue, ovarian tissue, breast tissue, liver tissue, or a combination thereof.

Embodiment 19. A pharmaceutical composition comprising an expression construct, wherein the expression construct comprises the UTR RNA sequence that has cell-type-specific activity specific to a target cell type and the UTR RNA sequence is obtained according to the method of embodiment 1, and a target gene, wherein the UTR RNA sequence is operably linked to the target gene.

Embodiment 20. A method for regulating expression of a target gene in a target cell, comprising administering the composition of embodiment 19 to the target cell, and inducing expression of the target gene in the target cell under suitable conditions.

### 8.5. Examples

The following examples are illustrative of the disclosure and should not be construed as limiting in any way the general nature of the disclosure of the description throughout this specification.

### Example 1—Computational and Experimental Discovery of RNA Structural Elements

### Systematic Annotation of Human RNA Structural Switches

The first goal of this study was to identify RNA sequences that can reside in two secondary structural conformations. For this, an integrative computational algorithm was developed that predicts whether an RNA sequence contains putative RNA structural switches. The computational algorithm satisfied several criteria: first, it should predict if a given RNA sequence contains a potential RNA switch and suggest the two mutually exclusive folding conformations. Secondly, it should be able to effectively capture a more generalizable definition of RNA switches in order to find instances beyond the 40 known RNA switch families (Kalvari et al. 2021). Thirdly, it should allow for seamless integration of experimental data to improve predictions. This is especially important as mRNA secondary structure in the cell is shown to be highly dynamic (Mortimer et al. 2014) and compartment-dependent (Sun et al. 2019); therefore, the predictions can be greatly improved with in vivo secondary structure probing data.

To discover new families of RNA switches, it was aimed to design an approach that does not rely on known sequence motifs, which has been the case for most published software (Wheeler and Eddy 2013; Nawrocki and Eddy 2013; Bengert and Dandekar 2004; Abreu-Goodger and Merino 2005; Chang et al. 2009; Mukherjee and Sengupta 2016). Instead, the method developed uses the sequence to generate an ensemble of secondary structures and their corresponding energy landscape using the Boltzmann ensemble concept (Ding and Lawrence 2003, incorporated herein by reference in its entirety). It then prioritizes those sequences that show RNA switch-like features, such as having two local minima in close proximity with a relatively small barrier in between. This approach ensures that RNA switches are described in a generalizable and family-agnostic way. It was demonstrated this point by holding individual Rfam families out of the training set and testing whether the method would predict the riboswitches correctly. High performance metrics was observed across all held-out families as measured by the Area Under the Receiver Operating Characteristic curve (AUROC) values. The performance of the method was further compared to SwiSpot, the state-of-the-art method for family-agnostic riboswitch prediction (Barsacchi et al. 2016, incorporated herein by reference in its entirety), and observed significant improvement of performance across most RNA switch families. By relying on biophysical features of the folding energy landscape as opposed to sequence features, the method captures a wider variety of RNA switches compared to the existing methods.

In addition to primary sequence, RNA secondary structure probing data, when available, was used to improve the method's predictions by updating the energy terms of the model. Eukaryotic genomes are large, therefore the models for RNA structural element prediction have to show very high specificity. Such specificity is difficult to reach by relying on in silico RNA folding alone, since RNAs can fold differently depending on the cell state (Beaudoin et al. 2018, incorporated herein by reference in its entirety). However, it is possible to achieve higher specificity if the RNA secondary structure is first probed in vivo and the model is then guided by this data. Therefore, the method was developed to have an option to update the energy landscape based on RNA secondary structure data (see Methods). The algorithm can use two approaches for modeling the RNA folding energy landscape, relying either on in silico RNA folding tools alone, or in tandem with experimental RNA secondary structure probing via methods like SHAPE-seq or DMS-MaPseq (REFS).

This functionality of the algorithm was used to improve the RNA switch predictions iteratively. First, the method was applied, using the naive in silico folding, to the entirety of 3′ untranslated regions (3′UTR) of the human transcriptome, and chose the top set of 3,750 top candidate switches (of the length <=186 nucleotides) as putative elements. RNA secondary structure probing was then performed for 3,750 candidate RNA switches in vivo using DMS-MaPseq applied to a massively parallel reporter system. The resulting data was used to improve the initial predictions. The top 1,461 high-confidence RNA structural switches were then carried forward from the second round of predictions for further functional and biochemical validation in vivo.

Discovery of RNA Switches with Regulatory Function in Human Transcriptome

In order to identify the RNA switches that are both functional and structurally bistable in the cell, two high-throughput in vivo screens were performed, i.e., the Structure Screen and the Functional Screen, respectively. The Structure Screen differentiates the RNAs that exist as an ensemble of two mutually exclusive conformations from those that reside in a single conformation only. The Functional Screen, in turn, measures the effect of candidate RNA switches on the expression of a reporter gene in parallel. Integrating data from the two screens together allows us to find, among the putative RNA switches, those that are regulatory active and act as switches in the cell.

### Large-Scale RNA Secondary Structure Probing for Improved RNA Switch Predictions

In order to improve the predictions of RNA switches, an in vivo RNA Structure Screen was performed, where it probed the secondary structure of 3,750 candidate switches in vivo using DMS-MaPseq (Zubradt et al. 2017). DMS preferentially modifies unpaired nucleotides resulting in substitutions during the reverse transcription process. Once the cDNA library is sequenced, the substitution frequency at a given position provides an estimate of nucleotide accessibility. Paired nucleotides typically have lower accessibility values compared to unpaired nucleotides. This method was applied to cells expressing the library of candidate RNA switches, enabling pooled and targeted accessibility measurement with single-nucleotides resolution across all 3,750 candidate RNA switches.

The Structure Screen data was used to identify bi-stable RNA structures. DMS-MaPseq measures the base reactivities of adenines and cytosines across all the coexisting RNA conformations in vivo. The reactivity of a single nucleotide is a population average of multiple RNA molecules that represent different minima in the RNA folding conformation ensemble. If one conformation dominates within the ensemble, it dominates the DMS-MaPseq reactivity profile; however, if multiple conformations co-exist, they all contribute to the reactivity profile (Morandi et al. 2021; Tomezsko et al. 2020; incorporated herein by reference in entireties). The method uses this difference to find the RNA switches that coexist in a balanced state between the two conformations. Therefore, the method, informed by nucleotide accessibility data, predicts not just the RNAs with the potential to act as RNA switches, but rather the RNAs that do act as RNA switches in vivo.

### Massively Parallel Reporter Assays for Identifying Regulatory RNA Switches

It was next sought to explore the potential role of the identified RNA switches in regulating gene expression. A massively parallel reporter assay was implemented to functionally interrogate RNA switches in vivo (Functional Screen). For this, it was tested whether a given RNA switch placed in a 3′ UTR can affect expression of its host mRNA, in this case eGFP, compared to a control scrambled sequence. A library of 3,750 candidate RNA switch sequences was cloned into a dual eGFP-mCherry fluorescent reporter vector, directly downstream of the eGFP coding frame. eGFP fluorescence was used to measure the effect of candidate RNA switches on gene expression, and mCherry fluorescence was used as an endogenous control. HEK293 cells were transduced with this synthetic library, used flow-cytometry to sort cells by eGFP/mCherry expression ratio, and sequenced the gDNA and RNA from the resulting 8 bins (see Methods). Of the candidate RNA switches tested, 536 (14%) showed significant downregulation relative to their scrambled control, and 538 (14%) showed a significant upregulation.

To annotate a high-confidence set of RNA switches with regulatory potential in the human transcriptome, a second iteration of predictions was performed, guided by the in vivo RNA structure probing data. To test the performance of this procedure, The fraction of regulatory active candidate RNA switches among the first and the second iterations of the prediction were compared, using the Functional Screen data. A higher fraction of regulatory active RNA switches among the second iteration of predictions compared to the first iteration (16% up- and 16% down-regulation versus 14% up- and 14% down-regulation) was observed. This supports the hypothesis that incorporating the in vivo RNA structure probing data improves its performance. The high-confidence RNA switches were integrated with the massively parallel reporter data. Together these analyses resulted in 1,461 elements that are significant in both screens (see methods).

### Massively Parallel Mutagenesis Analysis to Identify Conformation-Specific RNA Switch Activities

Having identified the candidate RNA switches that affect gene expression, it was aimed to assess the degree to which the two stable conformations show divergent regulatory function. For this, the MPRA was extended to include targeted mutations designed to shift the equilibrium between the two conformations of each riboswitch. This additional screen allowed us to identify bona fide RNA switches with strong conformation-dependent activity. Starting with the 1,461 high-confidence RNA switches described above, mutated variants were engineered that would lock RNA switches in one of their two predicted conformations. This was achieved by either disrupting or strengthening conformation-specific stem loops. A massively parallel reporter assay was then performed whereby each candidate RNA switch was represented by four additional conformation-specific variants (i.e., two vs. two). A total of 245 RNA switches were observed that change the reporter gene expression differently when locked in one or another conformation. The TCF7 RNA switch landscape has two local minima, corresponding to two alternative conformations supported by in vivo DMS-MaPseq data. Two mutations in different parts of the switching sequence that disfavor conformation 2 resulted in lower expression of eGFP reporter. Conversely, two mutations that disfavor conformation 1 increased eGFP expression. This observation indicates that the two conformations of the TCF7 RNA switch elicit divergent regulatory functions.

### Describing a Bi-Stable RNA Switch in 3′ UTR of RORC

Among the hundreds of conformation-specific RNA switches, the top performing element is chosen for further analysis. This riboswitch is located in 3′UTR of the RORC gene. The algorithm predicted a bistable secondary structure for this sequence. In this RNA switch, 5′ region can pair either with 3′ or with the middle region, leading to two mutually exclusive conformations. Our measurements indicate that this RNA switch exists in an equilibrium state between the two conformations in vivo, and that these two conformations have different effects on the expression of RORC.

In order to further confirm that the RORC RNA switch exists as an ensemble of two conformations, targeted mutagenesis experiments were performed with in vitro RNA SHAPE as the read-out. Mutation-rescue pairs of sequences were designed that first shift the equilibrium towards one conformation (mutation), and then shift it towards the opposite conformation (rescue). The accessibility of individual nucleotides was then measured using the in vitro SHAPE assay. It was observed that mutating 3′ region (117-AC), which is expected to stabilize conformation 2, reduced DMS accessibility of the middle region. Conversely, the rescue mutation (65-GT,117-AC) of 5′ region restored its wild-type accessibility. This finding further supports our model where the middle and 3′ region compete for base pairing to 5′ region. The complementary experiment showed a similar result. The mutation of the middle region (77-GA) is expected to stabilize the conformation 1. Even though a significant change in accessibility of 3′ region upon the 77-GA mutation was not observed, the rescue mutation (63-TC,77-GA), that was expected to stabilize the conformation 2, significantly increased the accessibility of 3′ region. Overall, in vitro SHAPE data supports the role of the three highlighted regions in forming an ensemble of two competing states.

To extend our in vitro observations to living cells, high-coverage DMS-MaPseq of the RORC switch was performed. A high DMS concentration sufficient to cause multiple conversions in the same RNA was used. This enabled us to cluster reads originating from alternative secondary structures using a state-of-the-art unsupervised computational approach, named DRACO (Morandi et al. 2021). In both biological replicates, DRACO identified two clusters, each representing one of the two conformations, at the approximate ratio of 27% to 73%.

Finally, to go beyond the secondary structure and gain insights into the 3D structure of this bistable switch, single-particle imaging (cryo-EM) of the wild-type RORC RNA switch was performed, and the conformation-specific sequence mutants 77-GA (stabilizing Conformation 1) and 117-AC (stabilizing Conformation 2) described above. Particles from these data separate into three structural classes labeled A, B, and C. The particles demonstrate RNA-like tertiary features, including apparent double-stranded helical segments with a discernible major groove, and typical RNA hairpin elements. The resolution of these reconstructions is limited to ˜10 Å, due to the extreme flexibility evinced by the raw micrographs and 2D class averages, but is sufficient for recognition of RNA folds. These data reveal that, as expected, the Class B structure is absent in the 77-GA mutant, while Class Cis absent in the 117-AC mutant. This indicates that the Class B structure pertains to Conformation 2, and Class C to Conformation 1. Interestingly, Class A is present in all three datasets. It was hypothesized that this structure represents a metastable intermediate that arises as the flexible RNA molecule dynamically refolds, and which is not itself destabilized by either of the two sequence mutants.

Comparing the cryo-EM structures to visualizations of DRACO secondary structure cluster representatives, it was proposed that the complex tertiary fold at the center of Class C is reflective of the multiple stem-loop motif at the center of the DRACO Conformation 2 representative, and which is exhibited across the cluster. It was further posited that the two legs of the hairpin-like Class B correspond to the two long helical segments of the DRACO Conformation 1 representative-again a feature exhibited in all members of that secondary structure cluster. Finally, the relatively simple inverted-L shape of Class A could primarily reflect the helix detected at the leading and trailing ˜20 residues in some members of either cluster. As these residues are identical in WT and mutant sequences, this assignment is consistent with the appearance of Class A in all three cryo-EM datasets.

Our findings indicate that RORC 3′ mRNA element landscape inhabits a shallow energy landscape with two rugged minima representing two major conformations. These minima are separated by a kinetic barrier that itself contains a metastable plateau reflecting a partially folded intermediate. This model is consistent with our computational prediction of multiple low-energy structures, experimental discovery of multiple secondary structure structures clusters via DMS-Seq/DRACO, and finally determination and functional assignment of multiple tertiary structures by cryo-EM and mutational analysis.

### The Alternative Conformations of the RORC RNA Switch Play Divergent Roles in Gene Regulation

Having discovered the RORC RNA switch as an ensemble of two conformations, the divergent activity of its two states is set out to be confirmed. For this, HEK293 cell lines stably expressing the conformation mutant sequences cloned into the eGFP 3′ UTR region were generated. The changes in eGFP expression of each mutant with a flow cytometer was then measured. Two parallel strategies to lock the RNA switch in the conformation 1 were employed: first, the middle region was mutated so that it cannot pair with 5′ region, and second, complementary mutations was introduced in both 5′ and 3′ regions, thus preventing the base pairing between 5′ and middle regions. These two orthogonal strategies achieve the same stabilization of secondary structures while modifying different parts of the sequence. Strikingly, both sets of modifications led to similar changes in eGFP expression: locking the RNA switch in the conformation 1 increased reporter gene expression compared to the wild type. Furthermore, the same two strategies were applied to instead lock the RNA switch in conformation 2 and observed the expected opposite effect: both modifications led to decrease of the reporter gene. Therefore, it is concluded that the two conformations play divergent functional roles.

It was aimed to test whether the secondary structure, rather than sequence composition, is the major determinant of the gene expression modulations. To do so, individual cell lines (as described above) stably expressing the mutant sequences from the rescue-mutation experiments was generated, and the effect of each mutant on gene expression measured. In total, 3 mutation-rescue pairs were tested. In all three cases, lower eGFP expression of the conformation X-stabilizing mutation 117-AC compared to the conformation X-stabilizing mutation 77-GA mutant cell line were observed. Overall, the reciprocal mutation-rescue experiments support the defining role of secondary structure in the functional effects of RORC RNA switch.

Next, whether shifting the equilibrium between the two conformations by trans-acting agents, rather than sequence mutations, would have an effect on gene expression was investigated. It was hypothesized that adding an antisense oligonucleotide (ASO) complementary to a part of the RNA switch sequence could shift the equilibrium between the two conformations to change reporter gene expression. A set of ASOs targeting the middle region of the switch was designed. These ASOs were designed to shift the equilibrium towards conformation 1 and thereby increase eGFP expression. The stable cell line expressing the RNA switch-containing reporter with ASOs was transfected and the changes in eGFP expression with a flow cytometer was measured. To ensure this effect is not specific to a single ASO design, the oligonucleotide chemistry was varied by using either morpholino, or 2′-O-(2-Methoxyethyl) (2-MOE) oligoribonucleotides, or locked nucleic acids (LNA) as the key modifications. Additionally, the sequence length and the frequency of modifications was varied. In all cases, transfecting cells with an ASO targeting the middle region of the RNA switch resulted in higher eGFP expression compared to a non-targeting ASO of the same chemistry and nucleotide composition. Thus, the repressive activity of the RORC RNA switch can be alleviated with the use of trans agents such as ASOs.

The RORC gene encodes nuclear receptor ROR-gamma, and has two protein isoforms that differ by a short N-terminal sequence. The shorter isoform, RORγ, is expressed in many tissues, and is involved in circadian rhythms. The longer isoform, RORγt, is expressed in several subsets of T cells and some lymphoid cells, and is a key driver of Th17 cell type differentiation (Eberl 2017). Therefore, the activity of the RORC RNA switch in its native context was also measured. It was assessed whether the conformation-dependent regulatory functions of the RORC RNA switch was maintained in the native Th17 cell context. Primary CD4+ cells with lentivirus carrying the double reporter and a sequence of interest in the eGFP 3′ UTR were infected. The cells were then differentiated into the Th17 cell type (Montoya and Ansel 2017). Inserting the wild type sequence of the RORC RNA switch strongly decreases the eGFP expression compared to a scrambled version of the same sequence. On the other hand, the 77-GA mutant decreases the strength of the repression, confirming the activity of the RORC RNA in Th17 cells.

### Genome-Wide CRISPRi Screens Reveal Pathways Downstream of the RORC RNA Switch

To explore the molecular mechanisms underlying the RORC switch's effect on gene expression, two distinct genome-wide CRISPRi screens were performed. The first screen was designed to identify trans factors epistatic to the repressive function of the RORC switch. For this, an eGFP construct carrying the RORC RNA switch in its 3′ UTR was used to measure the impact of every gene knockdown on the activity of this switch. The second screen, in contrast, was aimed at conformation-dependent activity by using a 77-GA mutant (77-GA) reporter instead. Considering the importance of RORC in T cell biology, the Jurkat T cell leukemia line was chosen as the model system for this screen. Both reporter cell lines were infected with a lentiviral genome-wide CRISPRi sgRNA library (Gilbert et al. 2014), sorted cells on a flow cytometer by eGFP expression, and the 25% of cells were collected with highest and lowest expression (de Boer et al. 2020). It was hypothesized that knocking down a gene important for repressive activity of the RORC RNA switch would result in higher expression of the reporter gene; therefore, genes whose targeting sgRNAs were enriched in the high reporter expression bin relative to those in the lower bins were searched for. Similarly, silencing genes responsible for the functional difference between the two conformations would result in a shift towards higher reporter expression shift for wildtype cell line compared to the 77-GA mutant cell line.

To identify factors responsible for the repressive function of RORC RNA switch, the abundance of sgRNAs in the cells was compared with high reporter expression relative to those with lower expression. Gene-set enrichment analysis was also performed (Korotkevich et al. 2021) to identify the key pathways involved. The most highly enriched pathway was nonsense-mediated decay (NMD) and several core NMD factors, such as SMG8, UPF1, UPF2, UPF3B were among the highest scoring hits of the screen. As expected, among other enriched pathways, many were associated with general gene expression such as translation, ribosome biogenesis, and endoplasmic reticulum stress. Next, it was asked which factors are responsible for the divergent activity of the two conformations. For this, the ratio of ratios test (DESeq2 testing ratio of ratios (RIP-S . . . ) was performed, by comparing the ratios of sgRNA abundance in low and high expression cells between the two reporter screens: wildtype versus 77-GA mutant. Interestingly, this comparison also highlighted the NMD pathway as the key contributor. Among the highest scoring hits were several core NMD factors; all of them are part of the SURF complex, which is thought to recognize stalled ribosomes in case the premature termination codon (PTC) happens upstream of the exon-junction complex (EJC) (Yamashita 2013). Therefore, it was hypothesized that the RORC RNA switch affects gene expression by acting through the EJC-independent NMD pathway, and that one conformation of the switch is more likely to act through this mechanism than the other.

To confirm this observation, CRISPRi was used to knock down the core NMD factors one at a time in the wild type and 77-GA mutant reporter lines, along with cells expressing a scrambled control (“scrambled” cell line). It was first asked whether knocking down the NMD factors would affect the repressive function of the RNA switch. The expression of eGFP in the wild type was compared with scrambled reporter lines. It was observed that silencing the core members of the SURF complex, but not of the EJC complex, affected the repressive effect of the RORC RNA switch sequence on gene expression. Next, it was asked whether knocking down the NMD proteins would reduce the functional difference between the two conformations of the switch. The expression of eGFP in the wild type was compared with the 77-GA mutant reporter lines. Consistently, it was observed that knocking down the core members of the SURF complex (Data not shown), and not of the EJC complex (data not shown), reduced this difference. This data demonstrates that NMD acts preferentially on the Conformation 2 of the RORC RNA switch.

The canonical NMD pathway causes the proteins translated from aberrant mRNA to be degraded by the proteasome (Kuroha et al. 2009). Therefore, it was expected that the RORC RNA switch to target its gene product for degradation by the proteasome through NMD pathway recruitment. To confirm this, two dual reporter cell lines were treated with the proteasome inhibitor bortezomib, one line expressing the RORC RNA switch (the “wild type” line), and the other expressing the scrambled control. As expected, proteasome inhibition resulted in a significantly larger change in eGFP expression in the “wild-type” line relative to the “scrambled” cell line. To verify that the observed effect is due to proteasome inhibition rather than a side effect of bortezomib, cells were treated with carfilzomib, another proteasome inhibitor which acts through a different mechanism, and observed the same effect as with bortezomib. Therefore, our data suggests that proteasomal degradation is involved in the repressive activity of the RORC RNA switch on gene expression.

Taken together, our results reveal the following mechanism for RORC RNA switch function. The SURF complex recognizes Conformation 2 and not Conformation 1 of the switch. Recruitment of the SURF complex, in turn, leads to decreased gene expression through proteasome-mediated protein degradation, and possibly other mechanisms. Our findings also demonstrate that conformation-dependent modes of gene expression control may be ubiquitous in the human transcriptome. Regulatory information encoded in dynamic RNA structural elements adds an underexplored avenue of gene expression control with fundamental roles in health and disease.

### Discussion

Historically, RNA switch discovery has been tackled by one of two methods: comparative genomics analysis or biochemical experimentation. Comparative genomics analysis searches for conserved positions within non-coding RNA regions; it works well for identifying cis-regulatory elements in bacteria, such as RNA switches and transcription factor binding sites (Rodionov 2007). The biochemical approach involves measuring the affinity of a putative RNA switch to its ligands and analyzing the conformational change caused by the binding event. Both approaches were used to discover the first known RNA switches in bacteria (Epshtein et al. 2003) (Winkler et al. 2002). However, no algorithms have been specifically designed to search for RNA switches in eukaryotic transcriptomes. In Eukaryotes, mRNA secondary structure is highly dynamic. Multiple studies have shown that RNA structure vastly differs when measured in vitro vs in vivo (Rouskin et al. 2014), and that multiple cellular processes can rearrange mRNA secondary structures (Sun et al. 2019). Several studies have shown the functional importance of individual RNA structure rearrangements, such as RNA thermosensors (Shamovsky et al. 2006); however, to what extent do structural switches control gene expression in eukaryotes is yet to be explored. There are several reasons why models trained on bacteria cannot be readily applied to higher eukaryotes. First, the sequence search space is much larger in eukaryotic transcriptomes compared to their bacterial counterparts, hindering the application of pre-trained models due to high false-positive counts (Ureta-Vidal et al. 2003). Second, poor sequence conservation of many eukaryotic RNA regulatory elements limits the applicability of the comparative genomics analyses (Backofen et al. 2018; Leypold and Speicher 2021). Hence, the primary approach used for finding eukaryotic switches has been low-throughput biochemical characterization of candidate sequences (Breaker 2011; Serganov and Nudler 2013). Here, an integrative and comprehensive platform for studying RNA switches in eukaryotic transcriptomes is described.

Recent advances in genomic technologies was a key contributor in our ability to carry out this systematic search for RNA switches. The development of RNA secondary structure probing techniques, such as DMS-seq and SHAPE-seq, has enabled researchers to move from only measuring the averaged structures of the folding ensemble to sampling multiple alternative conformations (Tomezsko et al. 2020; Morandi et al. 2021, incorporated herein by reference in entireties). Moreover, recent advances in single-particle cryo-EM and computational modeling have enabled structure determination of 3D folds of some RNA molecules (Kappel et al. 2020), despite their small size and intrinsic flexibility. This opens up a prospect of studying the functional difference between alternative RNA conformations and their role in gene expression control. Here, the present disclosures provide a strategy for exploring this question in a systematic manner. It was observed that a large number of regulatory elements in the human transcriptome act as RNA switches. Capturing the regulatory grammar of RNA switches across the transcriptome is a key step towards a more complete understanding of post-transcriptional control of gene expression.

In this study, restrictive criteria were chosen for selection of RNA switches. It was required these elements be bistable in vivo, i.e., to populate two mutually exclusive structural conformations. However, this condition doesn't have to apply to all functional RNA switches in the cell. Some RNA switches could be bistable under specific conditions and in specific cell types. Thus, future studies will likely find a wider variety of RNA switches than those have been discovered in the present study under steady-state conditions.

Finally, RNA switches likely function through a variety of different mechanisms. The known examples of human RNA switch mechanisms include mutually exclusive binding of RBPs by two different conformations (Ray et al. 2009) and m6A modification-based switching (Liu et al. 2015). It was demonstrated a new RNA switch that acts through the NMD pathway. It is speculated that other RNA switches similarly tap into the repertoire of known RNA metabolic pathways. For example, it has recently been shown that specific RNA structures cause aberrant splicing in metastatic cancers through binding SNRPA1 (Fish et al. 2021). Identifying the regulatory programs that govern RNA secondary structure switching will lead to a mechanistic understanding of gene expression control. It is expected this work will provide a basis for future studies of RNA switches in other contexts ranging from development and differentiation to various models of human disease.

### Methods

### Cell Culture

All cells were cultured in a 37° C. 5% CO2 humidified incubator. The 293T cells (ATCC CRL-3216) were cultured in DMEM high-glucose medium supplemented with 10% FBS, glucose (4.5 g/L), L-glutamine (4 mM), sodium pyruvate (1 mM), penicillin (100 units/mL), streptomycin (100 μg/mL) and amphotericin B (1 μg/mL) (Gibco). The Jurkat cell line was cultured in RPMI-1640 medium supplemented with 10% FBS, glucose (2 g/L), L-glutamine (2 mM), 25 mM HEPES, penicillin (100 units/mL), streptomycin (100 μg/mL) and amphotericin B (1 μg/mL) (Gibco). All cell lines were routinely screened for mycoplasma with a PCR-based assay.

### CRISPRi-Mediated Gene Knockdown

Jurkat cells expressing dCas9-KRAB fusion protein were constructed by lentiviral delivery of pMH0006 (Addgene #135448) and FACS isolation of BFP-positive cells.

The lentiviral constructs were co-transfected with pCMV-dR8.91 and pMD2.D plasmids using TransIT-Lenti (Mirus) into 293T cells, following the manufacturer's protocol. Virus was harvested 48 hours post-transfection and passed through a 0.45 μm filter. Target cells were then transduced overnight with the filtered virus in the presence of 8 μg/mL polybrene (Millipore).

Guide RNA sequences for CRISPRi-mediated gene knockdown were cloned into pCRISPRia-v2 (Addgene #84832) via BstXI-BlpI sites. After transduction with sgRNA lentivirus, Jurkat cells were selected with 2 μg/mL puromycin (Gibco).

### Cryo-EM Sample Preparation and Data Collection

3.5 μl of target mRNA at an approximate concentration of 1.5 mg/mL was applied to gold, 300 mesh TEM grids with a holey carbon substrate of 1.2/1.3 μm spacing (Quantifoil). The grids were blotted with #4 filter papers (Whatman) and plunge frozen in liquid ethane using a Mark IV Vitrobot (Thermo Fisher), with blot times of 4-6 s, blot force of −2, at a temperature of 8° C. and 100% humidity. All grids were glow discharged in an easiGlo (Pelco) with rarefied air for 30 s at 15 mA, no more than 1 hour prior to preparation. Duplicate WT and mutant RNA specimens were imaged under different conditions on several microscopes as per Table Y; all were equipped with K3 direct electron detector (DED) cameras (Gatan), and all data collection was performed using SerialEM².

### Cryo-EM Image Processing

Dose-weighted and motion-corrected sums were generated from raw DED movies on-the-fly during data collection using UCSF MotionCor2³. Images from super-resolution datasets were downsampled to the physical pixel size before further processing. CTF estimation was performed in CTFFIND4⁴, followed by neural-net based particle picking in EMAN2⁵. 2D classification, ab initio 3D classification, and gold-standard refinement were done in cryoSPARC⁶. CTFs were then re-estimated in cryoSPARC and particles repicked using low-resolution (20 Å) templates generated from chosen 3D classes. Extended datasets were pooled where appropriate, and particle processing was repeated through gold-standard refinement as before.

### Reporter Vector Design and Library Cloning

First, mCherry ORF was cloned into the BTV backbone (Addgene #84771). Then, the vector was digested with MluI-HF and PacI restriction enzymes (NEB), with the addition of rSAP (NEB). The digested vector was purified with Zymo DNA Clean and Concentrator-5 kit

DNA oligonucleotide libraries (one for Functional Screen and one for Conformation Expression Change Screen) consisting of 7500 sequences total were synthesized by Agilent. The second strand was synthesized as follows: the library DNA was digested with BmtI, then a UMI-containing primer (sequence CTCTTTCCCTACACGACGCTCTTCCGATCTNNNNNNCTAG) (SEQ ID NO:1) was used to initiate the second strand synthesis by Klenow Fragment (3′→5′ exo-) (NEB). The library was digested with MluI-HF and PacI restriction enzymes (NEB) and run on a 6% TBE polyacrylamide gel. The band of the corresponding size was cut out and the gel was dissolved in the DNA extraction buffer (10 mM Tris pH 8, 300 mM NaCl, 1 mM EDTA). The DNA was precipitated with isopropanol. The digested DNA library and the digested vector were ligated with T4 DNA ligase (NEB). The ligation reaction was precipitated with isopropanol and transformed into MegaX DH10B T1R Electrocompetent Cells (Thermo Fisher). The library was purified with ZymoPURE II Plasmid Maxiprep Kit (Zymo). The representation of individual sequences in the library was verified by sequencing the resulting library on MiSeq instrument (Illumina).

### Massively Parallel Reporter Assay

The DNA library was co-transfected with pCMV-dR8.91 and pMD2.D plasmids using TransIT-Lenti (Mirus) into 293T cells, following the manufacturer's protocol. Virus was harvested 48 hours post-transfection and passed through a 0.45 μm filter. HEK293 cells were then transduced overnight with the filtered virus in the presence of 8 μg/mL polybrene (Millipore); the amount of virus used was optimized to ensure the infection rate of ˜20%. The infected cells were selected with 2 μg/mL puromycin (Gibco). Cells were harvested at 90%-95% confluency for sorting and analysis on a BD FACSaria II sorter. The distribution of mCherry to GFP ratios was calculated. For sorting a library into subpopulations, the population into 8 bins each containing 12.5% of the total number of cells is gated. A total of 1.2 million cells were collected for each bin to ensure sufficient representation of sequence in the population in two replicates each. For each subpopulation, gDNA and total RNA were extracted with the Quick-DNA/RNA Miniprep kit. gDNA was amplified by PCR with Phusion polymerase (NEB) using primers CAAGCAGAAGACGGCATACGAGAT (SEQ ID NO: 2)-i7—GTGACTGGAGTTCAGACGTGTGCTCTTCCGATCACTGCTAGCTAGATGACTAAAC GCG (SEQ ID NO:3) and AATGATACGGCGACCACCGAGATCTACAC (SEQ ID NO:4)
i5—
ACACTCTTTCCCTACACGACGCTCTTCCGATCTGTGGTCTGGATCCACCGGTCC (SEQ ID NO:5). Different i7 indices were used for 8 different bins, and different i5 indices were used for the two replicates. RNA was reverse transcribed with Maxima H Minus Reverse Transcriptase (Thermo Fisher) using primer
CTCTTTCCCTACACGACGCTCTTCCGATCTNNNNNNNNNNNTGGTCTGGATCCAC CGGTCCGG (SEQ ID NO:6). The cDNA was amplified with Q5 polymerase (NEB) using primers CAAGCAGAAGACGGCATACGAGAT (SEQ ID NO:7)-i7—GTGACTGGAGTTCAGACGTGTGCTCTTCCGATCCTGCTAGCTAGATGACTAAACG C (SEQ ID NO:8) and CAAGCAGAAGACGGCATACGAGAT (SEQ ID NO:9)-i5—
GTGACTGGAGTTCAGACGTGTGCTCTTCCGATCTTACCCGTCATTGGCTGTCCA (SEQ ID NO:10). Different i7 indices were used for 8 different bins, and different i5 indices were used for the two replicates. The amplified DNA libraries were size purified with the Select-a-Size DNA Clean & Concentrator MagBead Kit (Zymo). Deep sequencing was performed using the HiSeq4000 platform (Illumina) at the UCSF Center for Advanced Technologies.

### DMS-MaPseg

DMS-MaPseq was performed as described in (33). Briefly, HEK293 cells were incubated in culture with 1.5% DMS (Sigma) at room temperature for 7 minutes, the media was removed, and DMS was quenched with 30% BME. Total RNA from DMS-treated cells and untreated cells was then isolated using Trizol (Invitrogen). RNA was reverse transcribed using TGIRT-III reverse transcriptase (InGex) and target-specific primers. PCR was then performed to amplify the desired sequences and to add Illumina compatible adapters. The libraries were then sequenced on a MiSeq instrument using MiSeq micro kit v2, 300 cycles (Illumina). See Supplementary Table for oligo sequences used in library preparation.

Pear (v0.9.6) was used to merge the paired reads into a single combined read. The UMI was then removed from the reads and appended to read names using UMI tools (v1.0). The reads were then reverse complemented (fastx toolkit) and mapped to the amplicon sequences using bwa mem (v0.7). The resulting bam files were then sorted and deduplicated (umi_tools, with method flag set to unique). The alignments were then parsed for mutations (CTK). The mutation frequency at every position was then reported.

### SHAPE Chemical Probing of RNAs

Chemical probing and mutate-and-map experiments were carried out as described previously (Palka et al. 2020). Briefly, 1.2 pmol of RNA was denaturated at 95° C. in 50 mM Na-HEPES, pH 8.0, for 3 min, and folded by cooling to room temperature over 20 min, and adding MgCl2 to 10 mM concentration. RNA was aliquoted in 15 μL volumes into a 96-well plate and mixed with nuclease-free H2O (control), or chemically modified in the presence of 5 mM 1-methyl-7-nitroisatoic anhydride (1M7) (Turner et al. 2013), for 10 min at room temperature. Chemical modification was stopped by adding 9.75 μL quench and purification mix (1.53 M NaCl, 1.5 μL washed oligo-dT beads, Ambion), 6.4 nM FAM-labeled, reverse-transcriptase primer (/56-FAM/AAAAAAAAAAAAAAAAAAAAGTTGTTCTTGTTGTTTCTTT) (SEQ ID NO: 11), and 2.55 M Na-MES. RNA in each well was purified by bead immobilization on a magnetic rack and two washes with 100 μL 70% ethanol. RNA was then resuspended in 2.5 μL nuclease-free water prior to reverse transcription.

RNA was reverse-transcribed from annealed fluorescent primer in a reaction containing 1× First Strand Buffer (Thermo Fisher), 5 mM DTT, 0.8 mM dNTP mix, and 20 U of SuperScript III Reverse Transcriptase (Thermo Fisher) at 48° C. for 30 min. RNA was hydrolyzed in the presence of 200 mM NaOH at 95° C. for 3 min, then placed on ice for 3 min and quenched with 1 volume 5 M NaCl, 1 volume 2 M HCl, and 1 volume 3 M sodium acetate. cDNA was purified on magnetic beads, then eluted by incubation for 20 min in 11 μL Formamide-ROX350 mix (1000 μL Hi-Di Formamide (Thermo Fisher) and 8 μL ROX350 ladder (Thermo Fisher). Samples were then transferred to a 96-well plate in “concentrated” (4 μL sample+11 μL ROX mix) and “dilute” (1 μL sample+14 μL ROX mix) for saturation correction in downstream analysis. Sample plates were sent to Elim Biopharmaceuticals for analysis by capillary electrophoresis.

### T Cell Isolation, Transduction, and Th17 Cells Differentiation

Th17 cells were derived as described previously (Montoya and Ansel 2017). Plates were coated with 2 μg/mL anti-human CD3 (UCSF monoclonal antibody core, clone: OKT-3) and 4 μg/mL anti-human CD28 (UCSF monoclonal antibody core, clone: 9.3) in PBS with calcium and magnesium for at least 2 h at 37° C. or overnight at 4° C. with plate wrapped in parafilm. Human CD4+ T cells were isolated from human peripheral blood using Easy Sep human CD4+ T cell isolation kit (17952; STEMCELL) and stimulated in ImmunoCult-XF T cell expansion medium (10981; STEMCELL) supplemented with 10 mM HEPES, 2 mM L-glutamine, 100 μM 2-ME, 1 mM sodium pyruvate, and 10 ng/ml TGF-β. 24 h after T cell isolation and initial stimulation on a 96-well plate, 7 ul of lentivirus was added to each sample. After 24 h, the media was removed from each sample without disturbing the cells and replaced with 200 ul fresh media. After 48 h, cells were stimulated with 1.2 μM ionomycin, 25 nM PMA, and 6 μg/ml brefeldin-A, resuspended by pipetting, incubated for 4 h at 37° C., and harvested for analysis.

Analysis of Capillary Electrophoresis Data with HiTRACE

Capillary electrophoresis runs from chemical probing and mutate-and-map experiments were analyzed with the HiTRACE MATLAB package (Yoon et al. 2011). Lanes were aligned together, bands fit to Gaussian peaks, background subtracted using the no-modification lane, corrected for signal attenuation, and normalized to the internal hairpin control. The end result of these steps is a numerical array of reactivity values for each RNA nucleotide that can be used as weights in structure prediction.

### Example 2—mRNAs with Cell Type-Specific Activity and Design of PARADE AI Framework

mRNA therapeutics offer revolutionary capabilities for disease treatment by directing cells to produce therapeutic proteins. Yet, it remains a challenge to attain mRNA stability and cell type specificity. The present invention address this challenge through PARADE (Prediction And RAtional DEsign of RNA UTRs), a generative AI framework, to engineer untranslated RNA regions with desired cell type-specific activity. To this end, the activity of 60,000 5′ and 3′ UTRs was measured across 6 cell types and developed a computational framework for rational design of synthetic UTRs with enhanced cell type-specificity. The activity of 12,000 de novo-designed sequences was then validated in cell lines; these sequences demonstrated superior specificity and activity compared to those of existing RNA therapeutics. PARADE was showcased by encoding a hepatotoxic cargo CYP2E1 on an mRNA that is selectively active in T cells but not in liver cells, leading to reduced hepatotoxicity. Further, oncosuppressor-carrying mRNAs were designed with prolonged activity suppressing the growth of adenocarcinoma and neuroglioma xenografts in mice using PTEN-encoding mRNA. All in all, PARADE allows for optimizing RNA activity across diverse cell types, paving the way for precise RNA-based therapeutics with improved treatment safety and efficacy.

### Example 3—Predicting and Designing mRNAs with Cell Type-Specific Activity

Massively parallel reporter assays (MPRA) (Melnikov et al. 2012; incorporated herein by reference in its entirety) was used to directly measure the influence of untranslated regions (both 5′UTRs and 3′UTRs) on RNA activity-specifically, the amount of protein produced from a given RNA-without the variability introduced by transcriptional control. This method allowed us to generate a large-scale dataset covering multiple cell types, which provides a clearer view of post-transcriptional regulation by focusing solely on the untranslated regions' impact.

This dataset enabled training PARADE (Prediction And RAtional DEsign of RNA UTRs), a generative artificial intelligence (AI) framework designed to create UTR sequences to achieve cell type-specific mRNA stability and translation. In result, PARADE-generated synthetic RNA sequences attain higher cell type-specificity compared to naturally occurring sequences and existing RNA therapeutics.

PARADE's practical application was further demonstrated by designing mRNAs that are selectively active in different cell types. Particularly, UTRs was engineered for mRNA encoding the hepatotoxic gene product CYP2E1 to be active in T cells but not hepatocytes, effectively reducing hepatotoxicity. Additionally, PARADE was applied to suppress the growth of adenocarcinoma and neuroglioma xenografts in mice by delivering PTEN-encoding mRNA. Importantly, PARADE is generalizable and can be applied to design mRNA UTRs selectively active in a variety of cell types, providing a powerful tool for developing safer, more effective RNA-based therapeutics. By offering a method to design mRNAs with controlled cell type-specificity, PARADE opens new possibilities for enhancing the safety and efficacy of mRNA therapies.

### Example 4—Massively Parallel Reporter Assays Provide a Comprehensive Dataset of Cell Type-Specific UTR Activity

To assess the activity of UTRs on a large scale, 60,000 UTR fragments were selected from the human transcriptome and evaluated their impact on reporter gene expression in six different cell lines. At the onset, 2068 transcripts were identified with cell type-dependent variability in translation efficiency from published Ribo-Seq data (Gerashchenko et al. 2020; Myers et al. 2019; Matheson et al. 2022). The hypothesis was that these transcripts would be more likely to harbor cell type-specific regulatory elements. From this set of transcripts, 60,000 regions were randomly sampled from the respective 5′ and 3′ UTRs of these transcripts, creating a comprehensive sequence library, which was referred to as Library 1. The massively parallel reporter assay (MPRA) was used to evaluate the activity of these UTR fragments across six cell lines, estimating protein output from genomically integrated reporter constructs via flow cytometry (Oikonomou, Goodarzi, and Tavazoie 2014). Throughout this study, “activity” refers to the relative expression of the reporter gene, measured by the center of mass of normalized read counts across the four sorted bins

For 5′ UTRs, a library of 30,000 fragments (50 nucleotides long) was constructed and cloned upstream of the eGFP open reading frame (ORF) in a polycistronic eGFP-mCherry reporter (FIG. 1A). In this design, the eGFP and mCherry are both transcribed from a single promoter but are translated separately-eGFP in a cap-dependent manner and mCherry in a cap-independent manner. Importantly, this design allows to decouple the effects of 5′ UTR sequences on translation from those on transcription. While variations in 5′ UTR sequences can impact the translation of eGFP, they do not affect the cap-independent translation of mCherry, providing an internal control for transcriptional changes. These libraries were transduced into six cell lines representing different cell types: Jurkat and Nalm-6 (blood), SW-480 (colon), PA-1 (ovary), MDA-MB-231 (breast), and HepG2 (liver). The cells were sorted into four bins based on their eGFP/mCherry expression ratios and sequenced the DNA from the sorted pools to analyze UTR activity.

For 3′ UTRs, a parallel library of 30,000 fragments (240 nucleotides long) was constructed, which were cloned downstream of the eGFP ORF in a bidirectional eGFP-mCherry reporter (FIG. 1A). In this setup, eGFP and mCherry are produced from separate RNAs, so changes in the eGFP/mCherry ratio reflect how 3′ UTR sequences influence both translation and RNA stability. As with 5′ UTRs, we sorted the cells into four groups based on their eGFP/mCherry expression ratio, followed by sequencing to measure the activity of 3′ UTR sequences in different cell lines.

Next, the study evaluated whether known regulatory elements in 5′ and 3′ UTRs had the expected effects in the experiments (FIG. 1B). The effects of upstream open reading frames (uORFs) in 5′ UTRs, known to repress translation by interfering with normal ribosome scanning and preventing translation initiation on the primary ORF (Morris and Geballe 2000), was analyzed. The data agrees with the established knowledge that the presence of uORFs decreases translation activity; noteworthy, the presence of frame-shifted upstream start codons (uAUGs) had a stronger impact on activity compared to the in-frame uAUG (P<10⁻⁵⁰).

For 3′ UTRs, it was observed that the presence of downstream open reading frames (dORFs) in 3′UTRs slightly but significantly (P=2*10⁻³³), enhances the activity of the main ORF as shown previously (Q. Wu et al. 2020). The presence of AU-rich elements (AREs), associated with mRNA instability and decreased translation (Chen and Shyu 1995), decreased the sequence activity in the MPRAs (P<10⁻⁵⁰). Lastly, the influence of the hsa-let-7i miRNA, known to mediate post-transcriptional repression (Johnson et al. 2005) was assessed, and a corresponding reduction in average activity for sequences containing its seed sequence (P=2*10⁻¹⁹) was observed. These observations support the validity of the present approach not only in capturing the regulatory effects of these specific elements but also in reliably measuring the effects of RNA sequence on post-transcriptional regulation more broadly.

The next step was to examine whether the MPRA data revealed UTRs with cell type-specific activity. While previous studies have shown that 3′UTRs harbor cell type-specific regulatory elements (Floor and Doudna 2016), 5′UTRs have traditionally been considered less likely to contribute to cell type-specific expression patterns (Hair et al. 2023). From the data, it was found that the activity levels of individual sequences were generally well-correlated across cell lines: correlations between cell lines were, on average, only 25% lower than within-cell line replicates, with median R values of 0.636 for 5′ UTRs and 0.619 for 3′ UTRs, compared to 0.856 and 0.820 for replicates (Suppl. FIG. 1B). Despite this overall correlation, some sequences exhibited notable differences in activity between cell lines.

To identify patterns of cell type-specific activity, unbiased K-means clustering was applied to the sequence activity measurements. This revealed distinct clusters of sequences with non-uniform activity profiles across the tested cell lines (FIGS. 1C and 1D). Among the 60,000 sequences, NUM1 showed significant cell type specificity (ANOVA P<0.05). Notably, this pattern was observed for both 3′ and 5′ UTRs, suggesting that both regions contain determinants of cell type-specific activity. Collectively, the high-throughput MPRA data are consistent with the known regulatory elements in RNA and capture the cell type-specific activity profiles of a large set of sequences.

### Example 5—PARADE Accurately Predicts Sequence Activity of Both 5′ and 3′ UTRs and Identifies the Regulatory Elements

To identify the sequence elements responsible for cell type-specific activity, a new deep learning model, PARADE, was designed and trained using the newly generated MPRA data. PARADE is based on the LegNet architecture, which has demonstrated excellent performance in predicting DNA regulatory activity (Penzar et al. 2023, incorporated hereby by reference in its entirety). LegNet is a convolutional neural network inspired by EfficientNetV2, optimized for short regulatory DNA sequences. Instead of treating the prediction task as a regression of a single real value from a nucleotide sequence, LegNet reformulates the task as a soft-classification problem, predicting probability distributions across expression bins and deriving expression levels as expected values of these distributions.

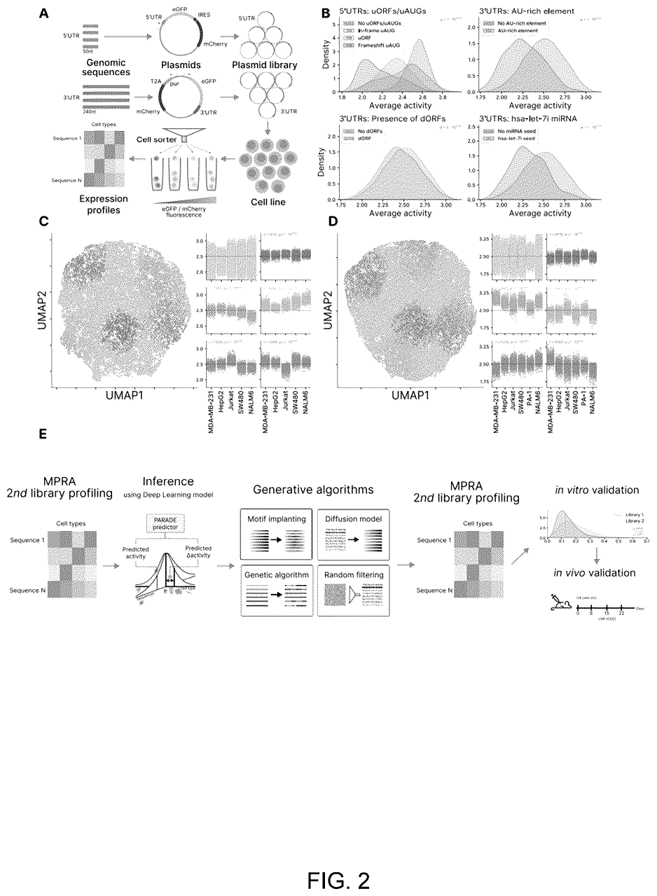

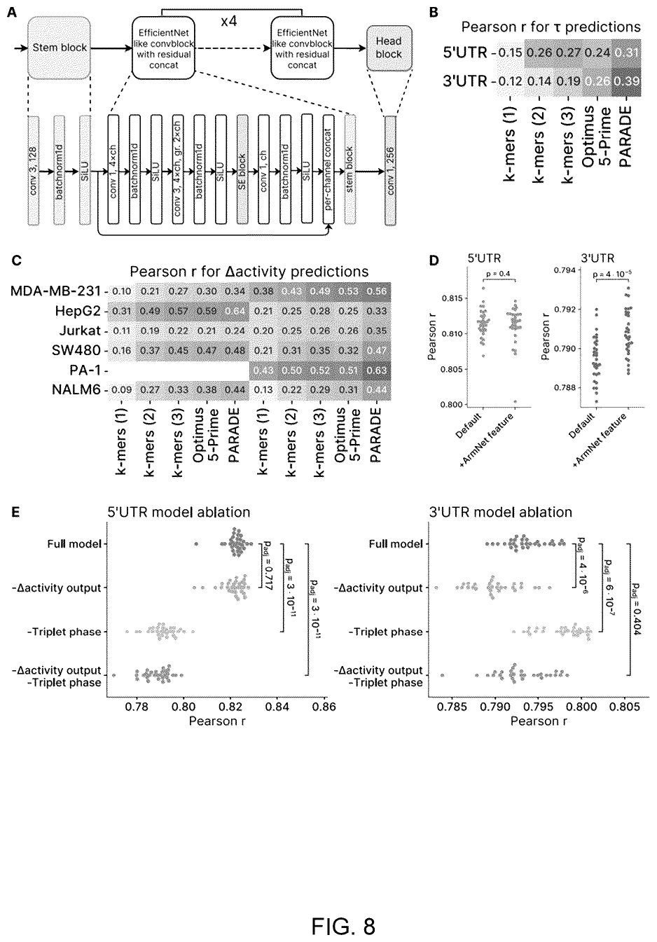

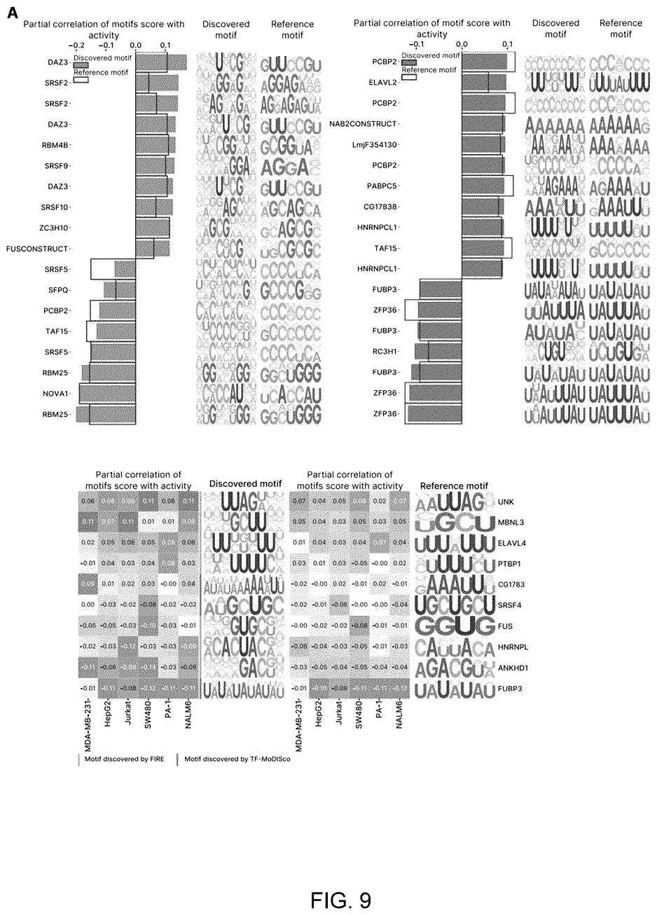

PARADE accurately predicts both the activity of a given sequence in a specific cell line and its “delta activity” Δ, i.e. the difference between its activity in that cell line and the average activity across all cell lines (FIG. 2A, FIG. 8A). PARADE outperformed both the regression of k-mer counts and the state of the art model Optimus-5-prime (Sample et al. 2019) across all cell lines (FIG. 2B), predicting cell type-specific sequence activity with Pearson correlations of 0.65-0.79 for 5′ UTRs and 0.65-0.75 for 3′ UTRs. The model performance was improved by providing additional sequence annotations, such as triplet phase (FIG. 8E). Notably, the triplet phase improved the performance of PARADE model when applied to 5′UTRs, but not 3′UTRs; conversely, “delta activity” only improved the performance of model on 3′UTRs.

To evaluate PARADE's ability to predict cell type-specific activity, the study focused on two metrics: the cell type specificity index t and the “delta activity” A (see above). The index t ranges from 0 to 1, where 0 represents ubiquitous expression (housekeeping genes) and 1 indicates exclusive expression in a single cell type (Yanai et al. 2005). Estimating from the cell type-specific predictions, PARADE accurately recovers the τ index for individual sequences, with a 30% and 50% improvement over Optimus-5-prime for 5′ UTRs and 3′ UTRs, respectively (Pearson correlation of 0.31 vs. 0.24 for 5′ UTRs and 0.39 vs. 0.26 for 3′ UTRs, FIG. 8B). Additionally, PARADE achieved higher accuracy in A prediction than both Optimus-5-prime and k-mer regression in every single cell line (FIG. 8C). Overall, these results highlight PARADE's superior capability in capturing cell type specific patterns of UTR sequence activity.

### Example 6—PARADE Identifies the Regulatory Elements Contributing to Cell Type Specific Activity

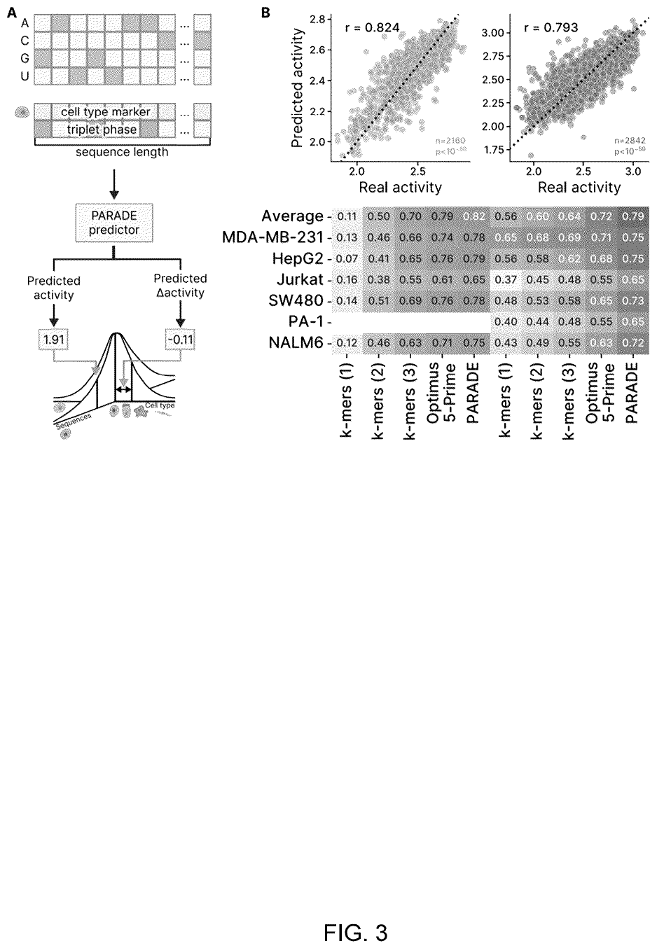

To uncover the regulatory elements driving cell type-specific UTR activity, motif analysis was performed using two complementary approaches. First, motif discovery was conducted directly on the MPRA data using FIRE (Elemento, Slonim, and Tavazoie 2007). Additionally, sequence patterns was analyzed the learned from the PARADE model using TF-Modisco (Avsec et al. 2021). While TF-Modisco did not identify any significant motifs in 5′ UTRs, both methods converged on a similar set of motifs in 3′ UTRs. These motifs were annotated by comparing them to known RNA binding protein (RBP) binding motifs (FIG. 3A).

To assess the contribution of individual motifs to UTR activity across different cell types, the correlations were calculated between motif scores and experimentally measured sequence activities, while controlling for nucleotide composition (see Methods). It was found that most, though not all, RBP binding motifs had varying degrees of influence on UTR activity depending on the cell line (FIG. 3A,B). For instance, the DAZAP1 motif in the 3′ UTR is associated with negative regulation in most cell lines, except in PA-1, an ovarian cancer cell line. This observation aligns with previous studies, which showed that DAZAP1 activates translation in oocytes when bound to 3′ UTR and poly-A tail of mRNA (R. W. P. Smith et al. 2011), but in other cells, like HEK293, it participates in mRNA degradation and silencing (H.-T. Yang et al. 2009).

The annotated motifs include both well-characterized regulators of post-transcriptional control and potentially novel regulatory elements. Among the known regulators, ZFP36 is a key factor in T cells, where it represses target mRNA expression during T-cell activation (Moore et al. 2018). Consistently, the presence of the ZFP36 motif in the 3′ UTR correlates with lower UTR activity, with the strongest effect observed in the Nalm-6 leukemia cell line. Similarly, ELAVL2, which promotes mRNA translation and P-body assembly in oocytes (Kato et al. 2019), shows a stronger association with UTR activation in PA-1 cells than in other cell lines. Finally, SRSF9, which stabilizes mRNA in colorectal cancer by interacting with m6A-modified regions (Wang et al. 2022), shows an association with 5′ UTR activity, particularly in the colorectal cancer cell line SW480 and the breast cancer cell line MDA-MB-231. Overall, these motif-activity associations not only demonstrate the biological relevance of the PARADE framework but also provide new insights into the regulatory roles of multiple RBPs.

### Example 7—PARADE Designs UTR Sequences with Desired Activity Levels and Cell Type-Specificity

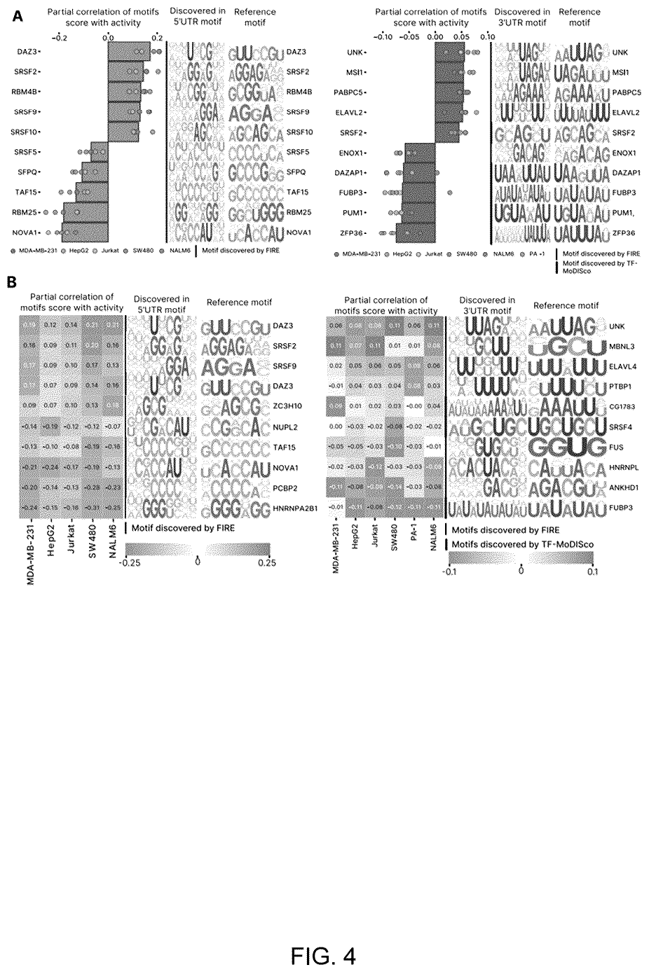

Building on PARADE's strong predictive accuracy for cell type-specific activity, several methods were implemented for generating RNA sequences with predefined activity levels and specificity (FIGS. 4A, B). In each method, it was sought to maximize the Cell Type Activity Difference (CTAD), a score that quantifies the difference in activity between two cell lines. Higher CTAD scores reflect greater cell type-specificity.

The first approach used was PARADE-Diffusion, a generative model based on the cold diffusion approach (Bansal et al. 2022, incorporated herein by reference in its entirety) (referred to as Diffusion). This method iteratively introduced mutations to generate de novo sequences, which were then evaluated by the PARADE predictor to select those with the highest CTAD. PARADE-Diffusion achieved correlations between desired and PARADE-predicted activity of 0.56-0.72 for both 5′ and 3′ UTRs (FIG. 4A). Importantly, over 99.8% of the sequences generated by PARADE-Diffusion were unique and distinct from the training set, demonstrating the model's capability to design novel sequences (Somepalli et al. 2022, incorporated herein by reference in its entirety).

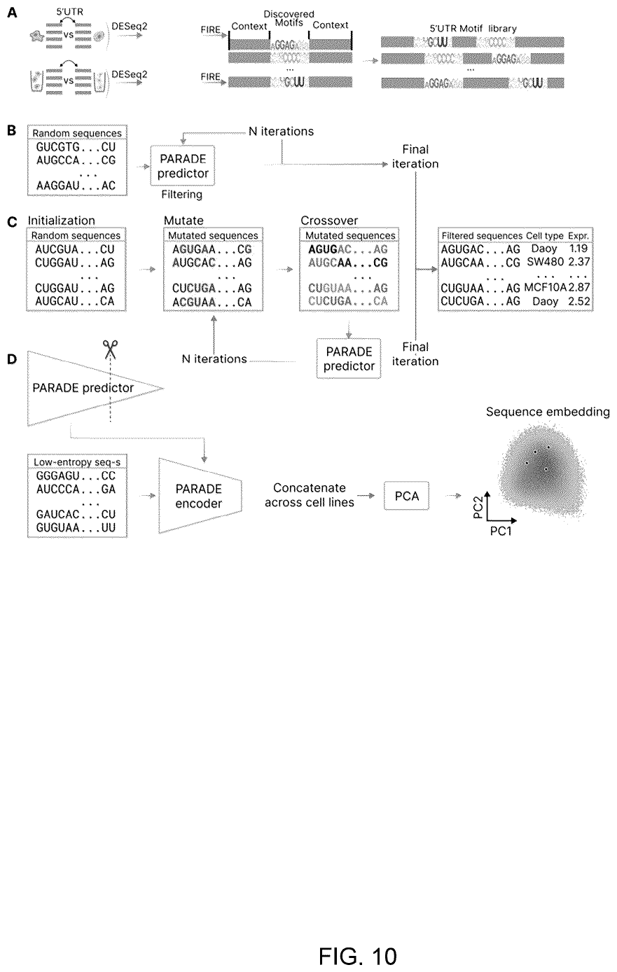

Next, a genetic algorithm (Kumar et al. 2010, incorporated herein by reference in its entirety) was implemented that directly optimized for CTAD by refining sequences to maximize the differential activity between cell lines (referred to as Genetic Algorithm; FIG. 10C). A random sampling approach (referred to as Random) was also employed, in which 10 million sequences were randomly generated, and the PARADE predictor was used to select those with the highest CTAD (FIG. 10B). Additionally, a motif-based design strategy (referred to as Motifs) was used, generating sequences based on combinations of previously identified regulatory motifs—two motifs for 5′ UTRs and four motifs for 3′ UTRs—from earlier analyses (FIG. 10A).

To validate the experimental setup, two control groups were included. The first control group consisted of characterized sequences from established RNA therapeutics, such as those used in Pfizer and Moderna's COVID vaccines, providing a benchmark for cell type-specificity. The second control group contained sequences with combinations of either activating or repressing motifs discovered in the motif analysis.

From all these methods and controls, 12,000 sequences were selected for 5′ UTR and another 12,000 sequences for 3′ UTR. This set of sequences, referred to as Library 2, was then tested using the MPRA assay in the same six cell lines (Jurkat, Nalm-6, SW-480, PA-1, MDA-MB-231, and HepG2).

### Example 8—Evaluation of Sequence Distribution in Multidimensional Space

It was hypothesized, and subsequently demonstrated, that generative AI tools could achieve higher cell type-specificity by exploring larger areas of the sequence space compared to naturally occurring RNA sequences, which are constrained by evolutionary pressure. To test this hypothesis, dimensionality reduction (PCA) was applied to the PARADE embeddings of approximately 1 million pseudorandom sequences (FIG. 10D). Naturally occurring UTR sequences were overlaid from the MPRA dataset (Library 1) on this space, alongside sequences generated by PARADE-Diffusion and the genetic algorithm.

For 5′ UTRs, both PARADE-Diffusion and genetic algorithm-generated sequences occupied a larger area of the multidimensional sequence space compared to the naturally occurring sequences, indicating that the models explored more diverse regions of the sequence space (FIG. 4C). For 3′ UTRs, diffusion-generated sequences covered a larger area than naturally occurring sequences, while the area explored by the genetic algorithm was smaller (FIG. 4D). These findings suggest that the AI models are capable of generating sequences that occupy a broader sequence space than those observed in nature.

It was also hypothesized that groups of sequences optimized for high CTAD between specific pairs of cell lines would cluster into distinct regions of the sequence space. Upon analyzing the distribution of these CTAD-optimized sequences generated by different algorithms, it was found that, in each case, these groups of sequences occupied distinct regions of the space. Interestingly, in some cases, the regions were identical across the generative algorithms (FIG. 4C), whereas in others, the regions differed (FIG. 4D). This observation highlights the potential for different generative models to explore alternative pathways for achieving high cell type-specificity.

### Example 9—Evaluation of Engineered RNA Sequence Activity and Specificity

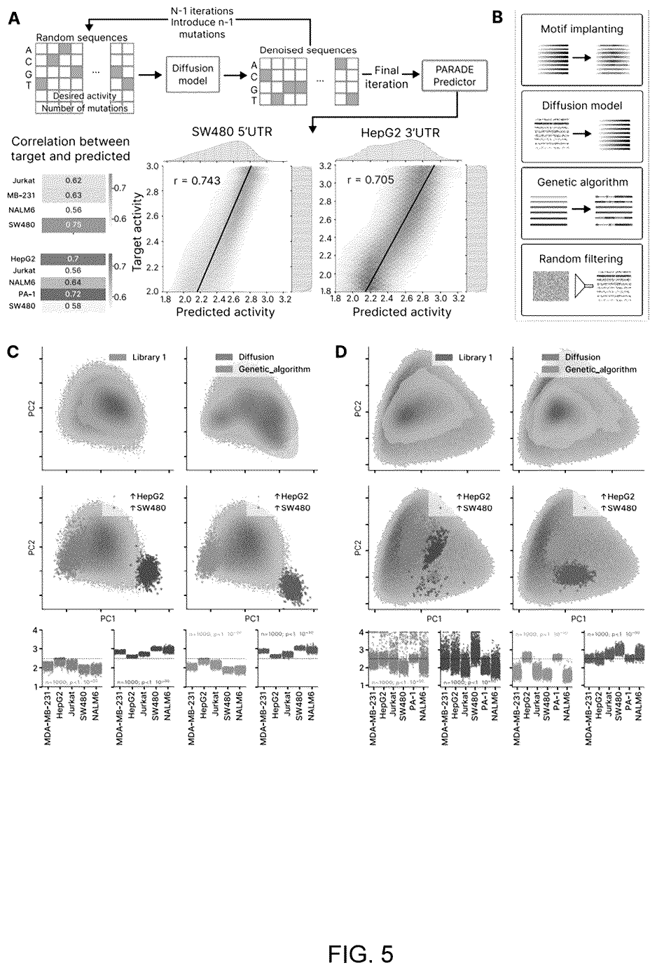

To evaluate the performance of the sequence generation algorithms, the tissue specificity of the designed sequences were experimentally measured. Library 2, consisting of 12,000 sequences designed by the four generative approaches—Diffusion, Genetic Algorithm, Random, and Motifs—and two control groups (characterized sequences and combinations of activating or repressing motifs), was constructed and tested using the MPRA assay in five cell lines (Jurkat, Nalm-6, SW-480, MDA-MB-231, HepG2). Compared to Library 1, Library 2 covered a larger area of the PARADE embedding space (FIG. 5A), indicating a greater diversity of sequences. The purpose of this evaluation was to assess four aspects: (1) the accuracy of PARADE in predicting sequence activity, (2) how closely the Diffusion method achieves the desired activity, (3) the improvement in cell type specificity compared to the controls and Library 1, and (4) the comparative performance of the generative methods in enhancing cell type specificity.

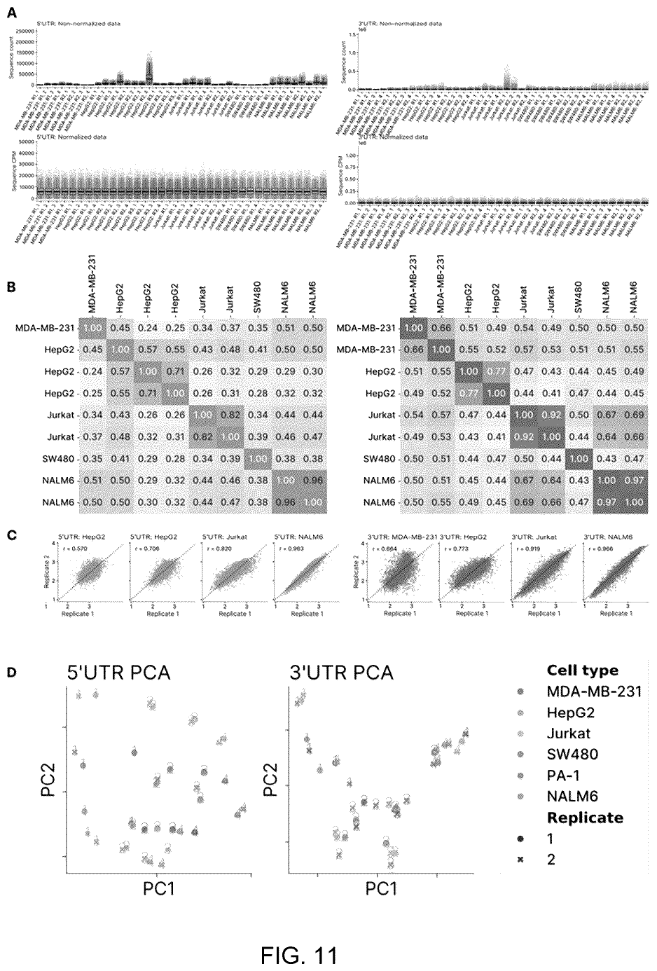

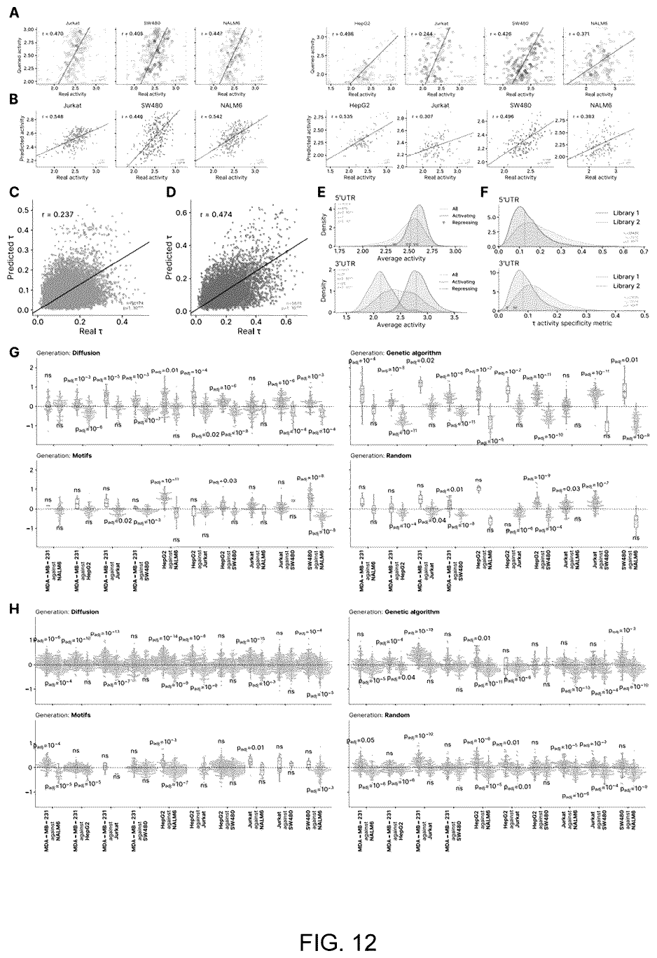

The first evaluation assessed PARADE's ability to predict sequence activity and the performance of the Diffusion method in generating sequences with the desired activity. The measured activities correlated well with PARADE's predictions, with R values ranging from 0.39 to 0.62 for 5′ UTR sequences and from 0.65 to 0.7 for 3′ UTR sequences (FIG. 5B), demonstrating the model's reliability in predicting sequence activity across diverse cell lines. For sequences designed using the PARADE-Diffusion method, the queried activity correlated with experimentally measured activity, ranging from 0.4 to 0.47 for 5′ UTRs and from 0.24 to 0.5 for 3′ UTRs (FIG. 11), showing that the Diffusion method generates sequences with activity levels close to the predicted values. Additionally, control sequences composed of combinations of activating or repressing motifs, identified through motif analysis, significantly increased or decreased baseline sequence activity (P-values of 2e⁻³⁷ and 5e⁻²⁰ for activating motif combinations in 5′UTRs and 3′UTRs, respectively, FIG. 11E). These results validated both the robustness of the assay and the motif discovery process used.

Next, the improvement in cell type specificity in Library 2 compared to Library 1 and the controls was assessed. The distribution of t values in Library 2 had a significantly higher mean than in Library 1, indicating a substantial increase in cell type specificity across the generated sequences (τ values of NUM2 and NUM3, for Library 1 and Library 2 respectively in 5′UTRs and of NUM4 and NUM5 in 3′UTRs, see FIG. 11F). Furthermore, PARADE's predictions of τ values correlated with experimentally measured τ values for 3′ UTRs (R=0.45), validating its capacity to predict cell type specificity (FIG. 11D). The sequences in Library 2 demonstrated cell type specificity that was on average NUM6 times higher than Pfizer's RNA therapeutics and NUM7 times higher than Moderna's, illustrating the potential for generating sequences with enhanced tissue specificity compared to existing RNA therapeutics (FIG. 11F).

Next, the performance of the generative methods with regard to the cell type specificity achieved was compared. Both for 5′ UTRs and 3′ UTRs, the deep learning-based methods—Genetic Algorithm, Diffusion, and Random—achieved significantly higher τ values than the Motif group (P-values of 5e-28, 4e-29, 4e-10 for 5′ UTRs, and 6e-55, 0.036, and 0.014 for 3′ UTRs, respectively; see FIG. 5C). The Genetic Algorithm group exhibited median NUM8-fold and NUM9-fold increases in τ values for 3′ UTRs compared to existing RNA therapeutics, including Moderna and Pfizer vaccines (FIG. 5C). Similar trends were observed for 5′ UTRs (FIG. 4C; FIG. 11C), although in this case, the Genetic Algorithm and Diffusion methods showed comparable τ values (median values of NUM10 and NUM11, respectively).

To further explore cell type-specific activity patterns, the sequences were grouped by their method of generation and by the pair of cell lines where PARADE maximized the CTAD. For each group, the measured activity difference was calculated between the target pair of sequences and assessed whether the observed difference was statistically significant using a single-ended t-test. Statistically significant differences were observed in 43/80 groups for 5′ UTR sequences and 41/80 groups for 3′ UTR sequences (FIGS. 5D, E). In all cases, the activity was higher in the desired cell line and lower in the opposite cell line (FIGS. 5F, G; FIGS. 11G, H).

For 3′ UTRs, Diffusion and Genetic Algorithm methods showed significant activity differences in the majority of cell line pairs (14/20 and 13/20 groups, respectively), while the Motif and Random groups showed significant differences in only 6 and 9 groups, respectively (FIG. 5E). Similarly, for 5′ UTRs, the Diffusion and Genetic Algorithm methods showed significant differences in 13 and 11 out of 20 cell line pairs, respectively, while the Random group performed similarly to Diffusion (13 out of 20), and the Motif group showed significant differences in only 7 out of 20 pairs (FIG. 5D, FIGS. 11G,H). These results demonstrate that the generative algorithms were able to achieve significant levels of cell type specificity, particularly for longer sequences such as 3′ UTRs.

### Example 10—Therapeutic Potential of Cell Type-Specific RNA Designs for Addressing Cargo Toxicity

A key goal of the study was to determine whether cell type-specific RNA designs generated by PARADE could address therapeutic challenges, such as cargo toxicity. To test this, CYP2E1 was selected, an enzyme whose overexpression sensitizes HepG2 cells to toxicity caused by glutathione depletion, as the model system (Bai and Cederbaum 2006; Mari and Cederbaum 2000). This cargo could be beneficial in other tissues but is harmful when expressed in the liver. This system was used to demonstrate the therapeutic potential of cell type specific RNA designs.

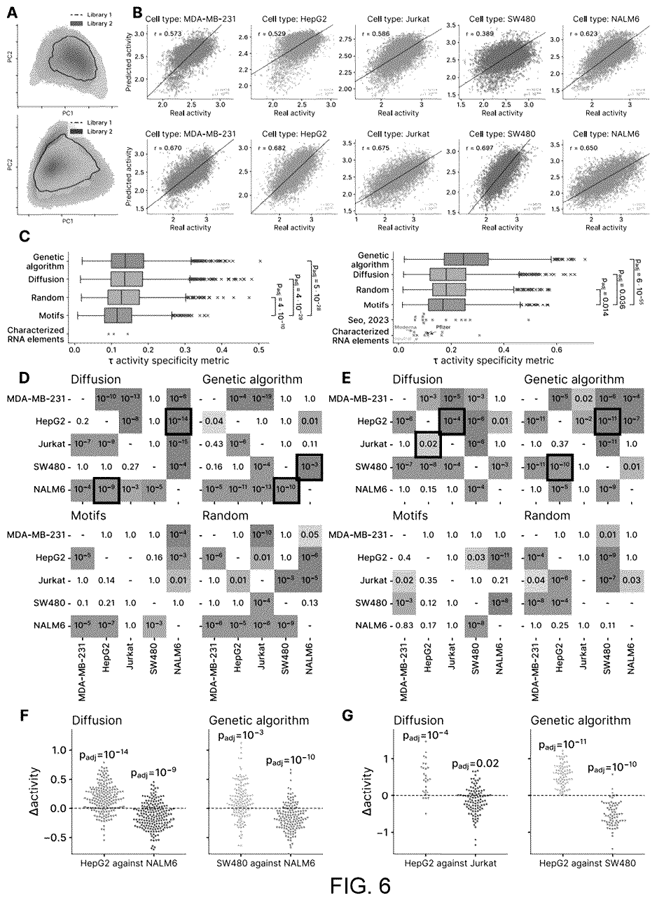

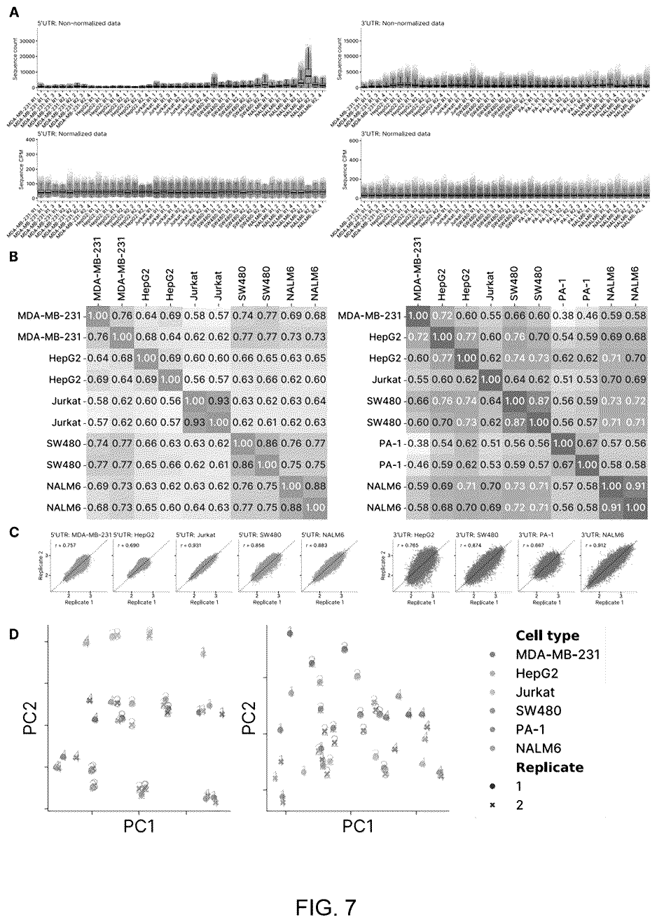

First, three sequences with high measured CTAD values between a T cell line (Jurkat) and a liver cell line (HepG2) were tested and their activity measured individually in primary human T cells and HepG2 cells. FireFly luciferase-encoding mRNA (Fluc) with the addition of PARADE-engineered UTRs were synthesized in vitro, transfected the cells by electroporation, and estimated RNA activity by measuring luminescence over three days (FIG. 6A). All three RNAs showed distinct activity patterns: RNA 1 demonstrated 5× higher activity in HepG2 cells, while RNAs 2 and 3 displayed 28× and 40× higher activity in T cells, respectively.

Next, to determine whether this specificity could reduce hepatotoxicity, CYP2E1-encoding mRNA was transfected using either commonly employed human beta-globin (HBB) UTRs or PARADE-designed cell type-specific UTRs. In HepG2 cells, CYP2E1 overexpression resulted in a 60% reduction in cell viability when combined with buthionine sulfoximine (BSO), a molecule that depletes glutathione. Importantly, when PARADE-designed UTRs with specificity for T cells (RNA 2 and RNA 3) were used, a significant increase in cell viability compared to the HBB UTRs—by 48% for RNA 2 (P=2.5×10⁻⁵) and by 41% for RNA 3 (P=2×10⁻⁴) was observed (FIG. 6B). No increased expression of CYP2E1 was detected in T cells, further demonstrating the specificity of these UTR designs (FIG. 6C). These results highlight the therapeutic potential of novel RNA designs to safely and specifically deliver toxic payloads, reducing the risk of off-target effects in undesired tissues, such as the liver.

### Example 11—Expanding PARADE's Capabilities for Enhanced mRNA Stability in Therapeutic Applications

Encouraged by PARADE's success in designing cell type-specific UTRs, it was sought to explore whether PARADE could also be adapted to address another key bottleneck in mRNA therapeutic design: enhancing mRNA stability. The PARADE model was fine-tuned on a relevant MPRA dataset that included measurements of both RNA and DNA counts for 3′ UTRs, using these as proxies for mRNA stability. PARADE demonstrated high performance on test datasets, with a correlation of NUM12, indicating strong predictive power for stability.

Two UTR sequences with high predicted stability (falling within the NUM13 and NUM14 quantiles) were selected and FireFly luciferase-encoding mRNAs (Fluc) synthesized using these PARADE-engineered UTRs. After transfecting T cells with these mRNAs, a significant X-fold increase in luciferase activity after three days was observed, compared to mRNAs using Moderna's UTRs. To further validate these findings in vivo, mice was injected with LNP-packaged mRNAs and observed an X-fold increase in stability compared to the Moderna UTRs.

Building on these findings, PTEN-encoding mRNA was designed using PARADE-optimized UTRs with enhanced stability. This mRNA was then tested for its ability to slow tumor growth in two distinct mouse models. In a xenograft model of PTEN-null tumors (PC3 cells), significant cytotoxicity upon delivery of PTEN-encoding mRNA was observed, reducing tumor growth (P=0.0011) and tumor weight (P=0.0067) compared to controls. Additionally, in an orthotopic model of neuroglioma, intratumoral delivery of PTEN-encoding mRNA through convection-enhanced delivery (CED) resulted in a marked reduction in tumor growth (P=0.0002) and a significant improvement in survival (P=0.034). These results demonstrate that PARADE's capability to design UTR sequences for enhanced mRNA stability can improve therapeutic outcomes, particularly in diseases with limited treatment options such as neuroglioma.

### Example 12—Rational Design of mRNAs with Cell Type-Specific Activity

In this study, generative AI was utilized to design UTR sequences that enhance cell type specificity and stability, key factors in improving the therapeutic effectiveness of mRNA. Combining large-scale MPRA data with the deep learning framework PARADE, RNA sequences were generated that outperform naturally occurring sequences and existing RNA therapeutics in terms of cell type specificity. The findings demonstrate that PARADE not only predicts RNA sequence activity with high accuracy but also generates novel UTR sequences capable of controlled expression across distinct cell types, addressing key challenges in mRNA-based therapeutics such as off-target effects and tissue toxicity.

### Generative Models Enhance Specificity Over Random and Physiological Sequences

The above study results show that generative AI methods-particularly Diffusion and Genetic Algorithm—achieve significantly better cell type specificity for longer sequences, such as 240 nt-long 3′ UTRs, than random sampling techniques. This finding aligns with the hypothesis that AI-driven models are more effective at exploring the complex multidimensional sequence space, especially where longer sequences allow for more regulatory interactions between motifs. However, for shorter sequences, such as 50 nt-long 5′ UTRs, the difference between the generative methods and random sampling was less pronounced, likely due to the limited scope for motif interactions in shorter regions.

These insights emphasize a broader challenge in RNA therapeutics: naturally occurring RNAs, shaped by evolutionary pressures, do not reach extreme levels of specificity due to the need for tight regulation and control of RNA-protein interactions. One explanation is that physiological RNAs are depleted of strong regulatory signals, leading to promiscuous and weak binding of functional RBPs, as demonstrated for C5 (Guenther et al. 2013). The specificity of RNA activity could be enhanced by learning the functional motifs from physiological RNAs, strengthening them, and combining them into novel combinations not present in living organisms, yet exhibiting extremely specific activity. Deep learning models are highly useful in this context, as they learn low-dimensional manifolds in sequence space, representing active regulatory motifs, and allow for their efficient manipulation (Koo and Ploenzke 2020). As demonstrated in FIG. 5C, the sequences generated by PARADE show significantly higher cell type-specificity than naturally occurring RNAs, underscoring the potential of generative models to overcome evolutionary constraints. While previous efforts have focused on designing UTRs to improve stability and translation efficiency using AI-based models (Castillo-Hair et al. 2024; Chu et al. 2024; Karollus, Avsec, and Gagneur 2021; Tang et al. 2024; Y. Yang et al. 2024; each of which is incorporated herein by reference in its entirety), the present invention extends these efforts by addressing cell type specificity, offering a more comprehensive solution for mRNA therapeutic design.

### Implications for RNA Therapeutics

PARADE's ability to design RNA sequences with enhanced tissue specificity and stability offers significant potential for advancing mRNA therapeutics. By tailoring these properties, PARADE can address key challenges in therapeutic mRNA design, such as reducing off-target effects and improving the stability of the RNA molecules. The combination of these attributes in future RNA designs could enable highly targeted therapies with increased efficacy and safety.

Looking ahead, PARADE could be applied to develop mRNA molecules that are both highly stable and tissue-specific. This would open up new therapeutic possibilities, such as targeting heart tissue with growth factor mRNA to promote recovery after a heart attack without causing unwanted effects in other tissues (Collén et al. 2022; incorporated herein by reference in its entirety). Similarly, more precise targeting of therapeutic mRNA for neurodegenerative diseases could reduce adverse effects in non-target areas of the brain (Sun and Roy 2021; incorporated herein by reference in its entirety).

### Methods

### Cell Culture

All cells were cultured in a 37° C. 5% CO2 humidified incubator. All cell lines insert cell line ATCC identifiers were cultured in RPMI-1640 medium supplemented with 10% FBS, glucose (2 g/L), L-glutamine (2 mM), 25 mM HEPES, penicillin (100 units/mL), streptomycin (100 μg/mL) and amphotericin B (1 μg/mL) (Gibco). All cell lines were routinely screened for mycoplasma with a PCR-based assay.

### Reporter Vector Design and Library Cloning

First, mCherry ORF was cloned into the BTV backbone (Addgene #84771). Then, the vector was digested with MluI-HF and PacI restriction enzymes (NEB), with the addition of rSAP (NEB). The digested vector was purified with Zymo DNA Clean and Concentrator-5 kit.

DNA oligonucleotide libraries (one for functional screen and one for massively parallel mutagenesis analysis) consisting of 7500 sequences total were synthesized by Agilent. The second strand was synthesized as follows: the library DNA was digested with BmtI, then a UMI-containing primer (sequence CTCTTTCCCTACACGACGCTCTTCCGATCTNNNNNNCTAG) (SEQ ID NO:12) was used to initiate the second strand synthesis by Klenow Fragment (3′→5′ exo-) (NEB). The library was digested with MluI-HF and PacI restriction enzymes (NEB) and run on a 6% TBE polyacrylamide gel. The band of the corresponding size was cut out and the gel was dissolved in the DNA extraction buffer (10 mM Tris pH 8, 300 mM NaCl, 1 mM EDTA). The DNA was precipitated with isopropanol. The digested DNA library and the digested vector were ligated with T4 DNA ligase (NEB). The ligation reaction was precipitated with isopropanol and transformed into MegaX DH10B TIR Electrocompetent Cells (Thermo Fisher). The library was purified with ZymoPURE II Plasmid Maxiprep Kit (Zymo). The representation of individual sequences in the library was verified by sequencing the resulting library on MiSeq instrument (Illumina).

### Massively Parallel Reporter Assay

The DNA library was co-transfected with pCMV-dR8.91 and pMD2.G plasmids using TransIT-Lenti (Mirus) into HEK293 cells, following the manufacturer's protocol. Virus was harvested 48 hours post-transfection and passed through a 0.45 μm filter. HEK293 cells were then transduced overnight with the filtered virus in the presence of 8 μg/mL polybrene (Millipore); the amount of virus used was optimized to ensure the infection rate of ˜20%. The infected cells were selected with 2 μg/mL puromycin (Gibco). Cells were harvested at 90%-95% confluency for sorting and analysis on a BD FACSaria II sorter. The distribution of mCherry to GFP ratios was calculated. For sorting a library into subpopulations, we gated the population into 8 bins each containing 12.5% of the total number of cells. A total of 1.2 million cells were collected for each bin to ensure sufficient representation of sequence in the population in two replicates each. For each subpopulation, gDNA and total RNA were extracted with the Quick-DNA/RNA Miniprep kit. gDNA was amplified by PCR with Phusion polymerase (NEB) using primers CAAGCAGAAGACGGCATACGAGAT (SEQ ID NO: 13)-i7—GTGACTGGAGTTCAGACGTGTGCTCTTCCGATCACTGCTAGCTAGATGACTAAAC GCG (SEQ ID NO:14) and AATGATACGGCGACCACCGAGATCTACAC (SEQ ID NO: 15)-i5—ACACTCTTTCCCTACACGACGCTCTTCCGATCTGTGGTCTGGATCCACCGGTCC (SEQ ID NO: 16). Different i7 indices were used for 8 different bins, and different i5 indices were used for the two replicates. RNA was reverse transcribed with Maxima H Minus Reverse Transcriptase (Thermo Fisher) using primer CTCTTTCCCTACACGACGCTCTTCCGATCTNNNNNNNNNNNTGGTCTGGATCCAC CGGTCCGG (SEQ ID NO:17). The cDNA was amplified with Q5 polymerase (NEB) using primers CAAGCAGAAGACGGCATACGAGAT (SEQ ID NO:18)-i7—GTGACTGGAGTTCAGACGTGTGCTCTTCCGATCCTGCTAGCTAGATGACTAAACG C (SEQ ID NO:19) and CAAGCAGAAGACGGCATACGAGAT (SEQ ID NO:20)-i5—GTGACTGGAGTTCAGACGTGTGCTCTTCCGATCTTACCCGTCATTGGCTGTCCA (SEQ ID NO:21). Different i7 indices were used for 8 different bins, and different i5 indices were used for the two replicates. The amplified DNA libraries were size purified with the Select-a-Size DNA Clean & Concentrator MagBead Kit (Zymo). Deep sequencing was performed using the HiSeq4000 platform (Illumina) at the UCSF Center for Advanced Technologies.

The adapter sequences were removed using cutadapt (Martin 2011). For RNA libraries, the UMI was then removed from the reads and appended to read names using UMI tools (T. Smith, Heger, and Sudbery 2017). The reads were matched to the fragments using bwa mem command. The reads were counted using featureCounts (Liao, Smyth, and Shi 2014). The read counts were normalized using median of ratios normalization (Anders and Huber 2010). One-way chi-square test statistic was used to estimate how different its distribution across the sorting bins is from the null hypothesis (i.e. uniform distribution). mRNA stability was estimated by comparing the RNA and DNA read counts with MPRAnalyze (Ashuach et al. 2019).

### REFERENCES

Almeida, Bernardo P. de, Christoph Schaub, Michaela Pagani, Stefano Secchia, Eileen E. M. Furlong, and Alexander Stark. 2024. “Targeted Design of Synthetic Enhancers for Selected Tissues in the Drosophila Embryo.” Nature 626 (7997): 207-11.
Anders, Simon, and Wolfgang Huber. 2010. “Differential Expression Analysis for Sequence Count Data.” Genome Biology 11 (10): R106.
Ashuach, Tal, David S. Fischer, Anat Kreimer, Nadav Ahituv, Fabian J. Theis, and Nir Yosef. 2019. “MPRAnalyze: Statistical Framework for Massively Parallel Reporter Assays.” Genome Biology 20 (1): 183.
Avsec, Žiga, Melanie Weilert, Avanti Shrikumar, Sabrina Krueger, Amr Alexandari, Khyati Dalal, Robin Fropf, et al. 2021. “Base-Resolution Models of Transcription-Factor Binding Reveal Soft Motif Syntax.” Nature Genetics 53 (3): 354-66.
Bai, Jingxiang, and Arthur I. Cederbaum. 2006. “Overexpression of CYP2E1 in Mitochondria Sensitizes HepG2 Cells to the Toxicity Caused by Depletion of Glutathione.” The Journal of Biological Chemistry 281 (8): 5128-36.
Balmayor, Elizabeth Rosado. 2022. “Synthetic mRNA-Emerging New Class of Drug for Tissue Regeneration.” Current Opinion in Biotechnology 74 (April): 8-14.
Bansal, Arpit, Eitan Borgnia, Hong-Min Chu, Jie S. Li, Hamid Kazemi, Furong Huang, Micah Goldblum, Jonas Geiping, and Tom Goldstein. 2022. “Cold Diffusion: Inverting Arbitrary Image Transforms Without Noise.” arXiv [cs.CV]. arXiv. http://arxiv.org/abs/2208.09392.
Barazandeh, Sina, Furkan Ozden, Ahmet Hincer, Urartu Ozgur Safak Seker, and A. Ercument Cicek. 2023. “UTRGAN: Learning to Generate 5′ UTR Sequences for Optimized Translation Efficiency and Gene Expression.” Bioinformatics. bioRxiv. https://www.biorxiv.org/content/10.1101/2023.01.30.526198v4.
Bateman, John F., Susanna Freddi, Gary Nattrass, and Ravi Savarirayan. 2003. “Tissue-Specific RNA Surveillance? Nonsense-Mediated mRNA Decay Causes Collagen X Haploinsufficiency in Schmid Metaphyseal Chondrodysplasia Cartilage.” Human Molecular Genetics 12 (3): 217-25.
Castillo-Hair, Sebastian, Stephen Fedak, Ban Wang, Johannes Linder, Kyle Havens, Michael Certo, and Georg Seelig. 2024. “Optimizing 5′UTRs for mRNA-Delivered Gene Editing Using Deep Learning.” Nature Communications 15 (1): 5284.
Chen, C. Y., and A. B. Shyu. 1995. “AU-Rich Elements: Characterization and Importance in mRNA Degradation.” Trends in Biochemical Sciences 20 (11): 465-70.
Chu, Yanyi, Dan Yu, Yupeng Li, Kaixuan Huang, Yue Shen, Le Cong, Jason Zhang, and Mengdi Wang. 2024. “A 5′ UTR Language Model for Decoding Untranslated Regions of mRNA and Function Predictions.” Nature Machine Intelligence 6 (4): 449-60.
Collen, Anna, Nils Bergenhem, Leif Carlsson, Kenneth R. Chien, Stephen Hoge, Li-Ming Gan, and Regina Fritsche-Danielson. 2022. “VEGFA mRNA for Regenerative Treatment of Heart Failure.” Nature Reviews. Drug Discovery 21 (1): 79-80.
Elemento, Olivier, Noam Slonim, and Saeed Tavazoie. 2007. “A Universal Framework for Regulatory Element Discovery across All Genomes and Data Types.” Molecular Cell 28 (2): 337-50.
Floor, Stephen N., and Jennifer A. Doudna. 2016. “Tunable Protein Synthesis by Transcript Isoforms in Human Cells.” eLife 5 (January). https://doi.org/10.7554/eLife.10921.
Gerashchenko, Maxim V., Mikhail V. Nesterchuk, Elena M. Smekalova, Joao A. Paulo, Piotr S. Kowalski, Kseniya A. Akulich, Roman Bogorad, et al. 2020. “Translation Elongation Factor 2 Depletion by siRNA in Mouse Liver Leads to mTOR-Independent Translational Upregulation of Ribosomal Protein Genes.” Scientific Reports 10 (1): 15473.
Gosai, S. J., R. I. Castro, N. Fuentes, J. C. Butts, S. Kales, R. R. Noche, K. Mouri, P. C. Sabeti, S. K. Reilly, and R. Tewhey. 2023. “Machine-Guided Design of Synthetic Cell Type-Specific Cis-Regulatory Elements.” Synthetic Biology. bioRxiv. https://biorxiv.org/content/10.1101/2023.08.08.552077v1.
Guenther, Ulf-Peter, Lindsay E. Yandek, Courtney N. Niland, Frank E. Campbell, David Anderson, Vernon E. Anderson, Michael E. Harris, and Eckhard Jankowsky. 2013. “Hidden Specificity in an Apparently Nonspecific RNA-Binding Protein.” Nature 502 (7471): 385-88.
Hair, Sebastian Castillo, Stephen Fedak, Ban Wang, Johannes Linder, Kyle Havens, Michael Certo, and Georg Seelig. 2023. “Optimizing 5′UTRs for mRNA-Delivered Gene Editing Using Deep Learning.” bioRxiv. https://doi.org/10.1101/2023.06.15.545194.
Jain, Ruchi, Josh P. Frederick, Eric Y. Huang, Kristine E. Burke, David M. Mauger, Elizaveta A. Andrianova, Sam J. Farlow, et al. 2018. “MicroRNAs Enable mRNA Therapeutics to Selectively Program Cancer Cells to Self-Destruct.” Nucleic Acid Therapeutics 28 (5): 285-96.
Johnson, Steven M., Helge Grosshans, Jaclyn Shingara, Mike Byrom, Rich Jarvis, Angie Cheng, Emmanuel Labourier, Kristy L. Reinert, David Brown, and Frank J. Slack. 2005. “RAS Is Regulated by the Let-7 microRNA Family.” Cell 120 (5): 635-47.
Karollus, Alexander, Ziga Avsec, and Julien Gagneur. 2021. “Predicting Mean Ribosome Load for 5′UTR of Any Length Using Deep Learning.” PLOS Computational Biology 17 (5): e1008982.
Kato, Yuzuru, Tokuko Iwamori, Youichirou Ninomiya, Takashi Kohda, Jyunko Miyashita, Mikiko Sato, and Yumiko Saga. 2019. “ELAVL2-Directed RNA Regulatory Network Drives the Formation of Quiescent Primordial Follicles.” EMBO Reports 20 (12): e48251.
Kim, Yoo-Ah, Kambiz Mousavi, Amirali Yazdi, Magda Zwierzyna, Marco Cardinali, Dillion Fox, Thomas Peel, Jeff Coller, Kunal Aggarwal, and Giulietta Maruggi. 2024. “Computational Design of mRNA Vaccines.” Vaccine 42 (7): 1831-40.
Koo, Peter K., and Matt Ploenzke. 2020. “Deep Learning for Inferring Transcription Factor Binding Sites.” Current Opinion in Systems Biology 19 (February): 16-23.
Kumar, Manoj, Dr Mohammad Husain, Naveen Upreti, and Deepti Gupta. 2010. “Genetic Algorithm: Review and Application.” https://doi.org/10.2139/ssrn.3529843.
Leppek, Kathrin, Gun Woo Byeon, Wipapat Kladwang, Hannah K. Wayment-Steele, Craig H. Kerr, Adele F. Xu, Do Soon Kim, et al. 2022. “Combinatorial Optimization of mRNA Structure, Stability, and Translation for RNA-Based Therapeutics.” Nature Communications 13 (1): 1536.
Liao, Yang, Gordon K. Smyth, and Wei Shi. 2014. “featureCounts: An Efficient General Purpose Program for Assigning Sequence Reads to Genomic Features.” Bioinformatics 30 (7): 923-30.
Liu. Yansheng. Andreas Beyer, and Rucdi Acbersold. 2016. “On the Dependency of Cellular Protein Levels on mRNA Abundance.” Cell 165 (3): 535-50.
Liu, Yue, Ian Hoskins, Michael Geng, Qiuxia Zhao, Jonathan Chacko, Kangsheng Qi, Logan Persyn, et al. 2024. “Translation Efficiency Covariation across Cell Types Is a Conserved Organizing Principle of Mammalian Transcriptomes.” Bioinformatics. bioRxiv. https://www.biorxiv.org/content/10.1101/2024.08.11.607360v1.
Ludwig, Nicole, Petra Leidinger, Kurt Becker, Christina Backes, Tobias Fehlmann, Christian Pallasch, Steffi Rheinheimer, et al. 2016. “Distribution of miRNA Expression across Human Tissues.” Nucleic Acids Research 44 (8): 3865-77.
Mari, M., and A. I. Cederbaum. 2000. “CYP2E1 Overexpression in HepG2 Cells Induces Glutathione Synthesis by Transcriptional Activation of Gamma-Glutamylcysteine Synthetase.” The Journal of Biological Chemistry 275 (20): 15563-71.
Martin, Marcel. 2011. “Cutadapt Removes Adapter Sequences from High-Throughput Sequencing Reads.” EMBnet.journal 17 (1): 10-12.
Matheson, Louise S., Georg Petkau, Beatriz Sáenz-Narciso, Vanessa D′Angeli, Jessica McHugh, Rebecca Newman, Haydn Munford, et al. 2022. “Multiomics Analysis Couples mRNA Turnover and Translational Control of Glutamine Metabolism to the Differentiation of the Activated CD4+ T Cell.” Scientific Reports 12 (1): 19657.
Melnikov, Alexandre, Anand Murugan, Xiaolan Zhang, Tiberiu Tesileanu, Li Wang, Peter Rogov, Soheil Feizi, et al. 2012. “Systematic Dissection and Optimization of Inducible Enhancers in Human Cells Using a Massively Parallel Reporter Assay.” Nature Biotechnology 30 (3): 271-77.
Metkar, Mihir, Christopher S. Pepin, and Melissa J. Moore. 2024. “Tailor Made: The Art of Therapeutic mRNA Design.” Nature Reviews. Drug Discovery 23 (1): 67-83.
Moore, Michael J., Nathalie E. Blachere, John J. Fak, Christopher Y. Park, Kirsty Sawicka, Salina Parveen, Ilana Zucker-Scharff, Bruno Moltedo, Alexander Y.
Rudensky, and Robert B. Darnell. 2018. “ZFP36 RNA-Binding Proteins Restrain T Cell Activation and Anti-Viral Immunity.” eLife 7 (May): e33057.
Morris, D. R., and A. P. Geballe. 2000. “Upstream Open Reading Frames as Regulators of mRNA Translation.” Molecular and Cellular Biology 20 (23): 8635-42.
Myers, Darienne R., Emilia Norlin, Yvonne Vercoulen, and Jeroen P. Roose. 2019. “Active Tonic mTORC1 Signals Shape Baseline Translation in Naive T Cells.” (ell Reports 27 (6): 1858-74.e6.
Obernosterer, Gregor, Philipp J. F. Leuschner, Mattias Alenius, and Javier Martinez. 2006. “Post-Transcriptional Regulation of microRNA Expression.” RNA 12 (7): 1161-67.
Oikonomou, Panos, Hani Goodarzi, and Saeed Tavazoie. 2014. “Systematic Identification of Regulatory Elements in Conserved 3′ UTRs of Human Transcripts.” Cell Reports 7 (1): 281-92.
Ong, Chin-Tong, and Victor G. Corces. 2011. “Enhancer Function: New Insights into the Regulation of Tissue-Specific Gene Expression.” Nature Reviews. Genetics 12 (4): 283-93.
Papadopoulos, Chris, Hugo Arbes, David Cornu, Nicolas Chevrollier, Sandra Blanchet, Paul Roginski, Camille Rabier, et al. 2024. “The Ribosome Profiling Landscape of Yeast Reveals a High Diversity in Pervasive Translation.” Genome Biology 25 (1): 268.
Penzar, Dmitry, Daria Nogina, Elizaveta Noskova, Arsenii Zinkevich, Georgy Meshcheryakov, Andrey Lando, Abdul Muntakim Rafi, Carl de Boer, and Ivan V. Kulakovskiy. 2023. “LegNet: A Best-in-Class Deep Learning Model for Short DNA Regulatory Regions.” Bioinformatics 39 (8). https://doi.org/10.1093/bioinformatics/btad457.
Plotkin, Joshua B., Harlan Robins, and Arnold J. Levine. 2004. “Tissue-Specific Codon Usage and the Expression of Human Genes.” Proceedings of the National Academy of Sciences of the United States of America 101 (34): 12588-91.
Riley, Aidan T., James M. Robson, and Alexander A. Green. 2023. “Generative and Predictive Neural Networks for the Design of Functional RNA Molecules.” Synthetic Biology. bioRxiv. https://www.biorxiv.org/content/10.1101/2023.07.14.549043v1.
Rohner, Eduarde, Ran Yang, Kylie S. Foo, Alexander Goedel, and Kenneth R. Chien. 2022. “Unlocking the Promise of mRNA Therapeutics.” Nature Biotechnology 40 (11): 1586-1600.
Sample, Paul J., Ban Wang, David W. Reid, Vlad Presnyak, Iain J. McFadyen, David R. Morris, and Georg Seelig. 2019. “Human 5′ UTR Design and Variant Effect Prediction from a Massively Parallel Translation Assay.” Nature Biotechnology 37 (7): 803-9.
Smith, Richard W. P., Ross C. Anderson, Joel W. S. Smith, Matthew Brook, William A. Richardson, and Nicola K. Gray. 2011. “DAZAP1, an RNA-Binding Protein Required for Development and Spermatogenesis, Can Regulate mRNA Translation.” RNA 17 (7): 1282-95.
Smith, Tom, Andreas Heger, and Ian Sudbery. 2017. “UMI-Tools: Modeling Sequencing Errors in Unique Molecular Identifiers to Improve Quantification Accuracy.” Genome Research 27 (3): 491-99.
Somepalli, Gowthami, Vasu Singla, Micah Goldblum, Jonas Geiping, and Tom Goldstein. 2022. “Diffusion Art or Digital Forgery? Investigating Data Replication in Diffusion Models.” arXiv [cs.I.G]. arXiv. http://arxiv.org/abs/2212.03860.
Sun, Jichao, and Subhojit Roy. 2021. “Gene-Based Therapies for Neurodegenerative Diseases.” Nature Neuroscience 24 (3): 297-311.
Tang, Xiaoshan, Miaozhe Huo, Yuting Chen, Hai Huang, Shugang Qin, Jiaqi Luo, Zeyi Qin, et al. 2024. “A Novel Deep Generative Model for mRNA Vaccine Development: Designing 5′ UTRs with N1-Methyl-Pseudouridine Modification.” Acta Pharmaceutica Sinica. B 14 (4): 1814-26.
Waldman, Yedael Y., Tamir Tuller, Tomer Shlomi, Roded Sharan, and Eytan Ruppin. 2010. “Translation Efficiency in Humans: Tissue Specificity, Global Optimization and Differences between Developmental Stages.” Nucleic Acids Research 38 (9): 2964-74.
Wang, Xiaoyu, Xiansheng Lu, Ping Wang, Qiaoyu Chen, Le Xiong, Minshan Tang, Chang Hong, et al. 2022. “SRSF9 Promotes Colorectal Cancer Progression via Stabilizing DSNI mRNA in an m6A-Related Manner.” Journal of Translational Medicine 20 (1): 198.
Wu, Han, Alex S. Nord, Jennifer A. Akiyama, Malak Shoukry, Veena Afzal, Edward M. Rubin, Len A. Pennacchio, and Axel Visel. 2014. “Tissue-Specific RNA Expression Marks Distant-Acting Developmental Enhancers.” PLOS Genetics 10 (9): e1004610.
Wu, Qiushuang, Matthew Wright, Madelaine M. Gogol, William D. Bradford, Ning Zhang, and Ariel A. Bazzini. 2020. “Translation of Small Downstream ORFs Enhances Translation of Canonical Main Open Reading Frames.” The EMBO Journal 39 (17): e104763.
Yanai, Itai, Hila Benjamin, Michael Shmoish, Vered Chalifa-Caspi, Maxim Shklar, Ron Ophir, Arren Bar-Even, et al. 2005. “Genome-Wide Midrange Transcription Profiles Reveal Expression Level Relationships in Human Tissue Specification.” Bioinformatics 21 (5): 650-59.
Yang, Huei-Ting, Mark Peggie, Philip Cohen, and Simon Rousseau. 2009. “DAZAP1 Interacts via Its RNA-Recognition Motifs with the C-Termini of Other RNA-Binding Proteins.” Biochemical and Biophysical Research Communications 380 (3): 705-9.
Yang, Yuning, Gen Li, Kuan Pang, Wuxinhao Cao, Zhaolei Zhang, and Xiangtao Li. 2024. “Deciphering 3′UTR Mediated Gene Regulation Using Interpretable Deep Representation Learning.” Advanced Science (Weinheim, Baden-Wurttemberg, Germany), August, e2407013.
Yin, Christopher, Sebastian Castillo Hair, Gun Woo Byeon, Peter Bromley, Wouter Meuleman, and Georg Seelig. 2024. “Iterative Deep Learning-Design of Human Enhancers Exploits Condensed Sequence Grammar to Achieve Cell Type-Specificity.” bioRxiv: The Preprint Server for Biology, June. https://doi.org/10.1101/2024.06.14.599076.
Zhang, He, Liang Zhang, Ang Lin, Congcong Xu, Ziyu Li, Kaibo Liu, Boxiang Liu, et al. 2023. “Algorithm for Optimized mRNA Design Improves Stability and Immunogenicity.” Nature 621 (7978): 396-403.
n.d. Accessed Oct. 22, 2024. https://hal.science/hal-04395528.

### 8.6. Additional Considerations

All references cited herein are incorporated by reference to the same extent as if each individual publication, database entry (e.g., Genbank sequences or GeneID entries), patent application, or patent, was specifically and individually indicated to be incorporated by reference in its entirety, for all purposes. This statement of incorporation by reference is intended by Applicants, pursuant to 37 C.F.R. § 1.57 (b) (1), to relate to each and every individual publication, database entry (e.g., Genbank sequences or GeneID entries), patent application, or patent, each of which is clearly identified in compliance with 37 C.F.R. § 1.57 (b) (2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

The foregoing description of the embodiments has been presented for the purpose of illustration; it is not intended to be exhaustive or to limit the patent rights to the precise forms disclosed. Persons skilled in the relevant art will appreciate that many modifications and variations are possible in light of the above disclosure.

Any feature mentioned in one claim category, e.g., method, can be claimed in another claim category, e.g., computer program product, system, storage medium, as well. The dependencies or references back in the attached claims are chosen for formal reasons only. However, any subject matter resulting from a deliberate reference back to any previous claims (in particular multiple dependencies) can be claimed as well, so that any combination of claims and the features thereof is disclosed and can be claimed regardless of the dependencies chosen in the attached claims. The subject matter will be understood to include not only the combinations of features as set out in the disclosed embodiments but also any other combination of features from different embodiments. Various features mentioned in the different embodiments can be combined with explicit mentioning of such combination or arrangement in an example embodiment or without any explicit mentioning. Furthermore, any of the embodiments and features described or depicted herein can be claimed in a separate claim and/or in any combination with any embodiment or feature described or depicted herein or with any of the features.

Some portions of this description describe the embodiments in terms of algorithms and symbolic representations of operations on information. These operations and algorithmic descriptions, while described functionally, computationally, or logically, are understood to be implemented by computer programs or equivalent electrical circuits, microcode, or the like. Furthermore, it has also proven convenient at times, to refer to these arrangements of operations as engines, without loss of generality. The described operations and their associated engines are, in some embodiments, embodied in software, firmware, hardware, or any combinations thereof.

Any of the steps, operations, or processes described herein, in some embodiments, are performed or implemented with one or more hardware or software engines, alone or in combination with other devices. In one embodiment, a software engine is implemented with a computer program product comprising a computer-readable medium containing computer program code, which can be executed by a computer processor for performing any or all of the steps, operations, or processes described. The term “steps” does not mandate or imply a particular order. For example, while this disclosure describes, in some embodiments, a process that includes multiple steps sequentially with arrows present in a flowchart, the steps in the process do not need to be performed by the specific order claimed or described in the disclosure. In some implementations, some steps are performed before others even though the other steps are claimed or described first in this disclosure. Likewise, any use of (i), (ii), (iii), etc., or (a), (b), (c), etc., in the specification or in the claims, unless specified, is used to better enumerate items or steps and also does not mandate a particular order.

Throughout this specification, plural instances may implement components, operations, or structures described as a single instance. Although individual operations of one or more methods are illustrated and described as separate operations, in some implementations one or more of the individual operations are performed concurrently, and nothing requires that the operations be performed in the order illustrated. Structures and functionality presented as separate components in example configurations are, in some embodiments, implemented as a combined structure or component. Similarly, in some embodiments, structures and functionality presented as a single component are implemented as separate components. These and other variations, modifications, additions, and improvements fall within the scope of the subject matter herein. In addition, the term “each” used in the specification and claims does not imply that every or all elements in a group need to fit the description associated with the term “each.” For example, “each member is associated with element A” does not imply that all members are associated with an element A. Instead, the term “each” only implies that a member (of some of the members), in a singular form, is associated with an element A. In claims, in some instances, the use of a singular form of a noun implies at least one element even though a plural form is not used.

Finally, the language used in the specification has been principally selected for readability and instructional purposes, rather than selected to delineate or circumscribe the patent rights. It is therefore intended that the scope of the patent rights be limited not by this detailed description, but rather by any claims that issue on an application based hereon. Accordingly, the disclosure of the embodiments is intended to be illustrative, but not limiting, of the scope of the patent rights.

Although inventions have been particularly shown and described with reference to a preferred embodiment and various alternate embodiments, it will be understood by persons skilled in the relevant art that various changes in form and details can be made therein without departing from the spirit and scope of the invention.

What is claimed is:

1. A method for predicting cell-type-specific activity of an untranslated region (UTR) RNA sequence, comprising:
A) obtaining UTR RNA sequence data corresponding to a plurality of UTR RNA sequences;
B) processing the UTR RNA sequence data using a model, wherein the model is configured to, for each respective UTR RNA sequence in the plurality of UTR RNA sequences:
predict a cell-type-specific activity of the respective UTR RNA sequence for each target cell type in a plurality of target cell types, and/or
predict a delta activity of the UTR RNA sequence for each target cell type in the plurality of target cell types, wherein the delta activity is defined as difference between the predicted cell-type-specific activity specific to the respective target cell type and an average activity across the plurality of target cell types; and
C) outputting, for each respective UTR RNA sequence in the plurality of UTR RNA sequences, a predicted set of metrics for the cell-type-specific activity and/or the delta activity for each target cell type in the plurality of target cell types.

2. The method of claim 1, wherein the cell-type-specific activity of an UTR RNA sequence quantifies effect of the UTR RNA sequence on translation and/or mRNA stability of a target gene under suitable conditions, when the UTR RNA sequence and the target gene are operably linked in an expression construct.

3. The method of claim 1, further comprising validating the outputted sets of metrics with one or more in vitro or in vivo assays, and feeding validation data obtained from the one or more in vitro or in vivo assays back to the model, thereby improving accuracy of prediction by the model

4. The method of claim 3, comprising validating the outputted sets of metrics with a Massively Parallel Reporter Assay (MPRA).

5. The method of claim 1, wherein the model is trained with a Massively Parallel Reporter Assay (MPRA) dataset to predict cell-type-specific activity and delta activity of RNA sequences corresponding to one or more target cell types, wherein the MPRA dataset comprises:
the plurality of UTR RNA sequences, and
for each respective UTR RNA in the plurality of UTR RNA sequences, measurements of corresponding cell-type-specific activity specific to the one or more target cell types, measured from the MPRA.

6. The method of claim 5, wherein the plurality of target cell types comprise cells from one or more tissues selected from the group consisting of blood tissue, colon tissue, ovarian tissue, breast tissue, and liver tissue.

7. The method of claim 1, wherein the plurality of UTR RNA sequences in the MPRA dataset comprises at least 1,000, at least 10,000, at least 100,000, or at least 1×10⁶ 5′ UTR RNA sequences; or at least 1,000, at least 10,000, at least 100,000, or at least 1×10⁶ 3′ UTR RNA sequences.

8. The method of claim 1, wherein each UTR RNA sequence in the plurality of the UTR RNA sequences comprises at most 50, at most 100, at most 150, at most 200, at most 250, at most 300, at most 350, at most 400, at most 450, or at most 500 nucleotides.

9. The method of claim 1, wherein step A) further comprises obtaining for each respective UTR RNA sequence in the plurality of UTR RNA sequences, additional sequence information comprising triplet phase information for each respective UTR RNA sequence.

10. The method of claim 1, wherein, specific to each target cell type in the plurality of target cell types, the outputted set of metrics comprises:
a cell type specificity index (τ), for each respective UTR RNA sequence in the plurality of UTR RNA sequences, wherein τ:
ranges from 0 to 1,
indicates ubiquitous activity at 0, and
indicates exclusive activity in a single cell type at 1; and
a delta activity value (α) quantifying difference between the predicted cell-type-specific activity and an average activity across the plurality of target cell types.

11. The method of claim 1, wherein the model comprises a LegNet model.

12. The method of claim 1, further comprising regulating expression of a target gene in a target cell, comprising:
operably linking an UTR RNA sequence to the target gene in an expression construct, wherein the UTR RNA sequence exhibits cell-type-specific activity when the expression construct is transferred to a corresponding target cell type and under suitable conditions; and
transferring the expression construct to the target cell.

13. A method for predicting regulatory motifs in an UTR RNA sequence, comprising:
A) obtaining UTR RNA sequence data for each respective UTR RNA sequence in a plurality of UTR RNA sequences, and associated activity for each respective UTR RNA sequence;
B) processing the UTR RNA sequence data using a model, wherein the model is configured to, for each respective UTR RNA sequence in the plurality of UTR RNA sequences, identify one or more candidate motifs in each respective UTR RNA sequence;
C) determining, for each respective UTR RNA sequence in the plurality of UTR RNA sequences, correlation of each candidate motif identified sequences with the associated UTR RNA activity; and
D) outputting a ranked list of candidate motifs identified in the plurality of UTR RNA sequences, wherein the ranking is based on correlation between each respective candidate motif and UTR activity.

14. A method for generating a plurality of UTR RNA sequences with predefined cell-type-specific activity, comprising:
A) obtaining input data comprising one or more predefined activity and one or more cell type-specificity constraints;
B) processing the input data using a first portion of a model to generate a plurality of UTR RNA sequences, wherein the first portion of the model is configured to refine the UTR RNA sequences based on the one or more predefined activity and the one or more cell type-specificity constraints;
C) responsive to step B), using a second portion of the model to predict, for each respective UTR RNA sequence in the plurality of UTR RNA sequences generated in step B):
a cell-type-specific activity of the respective UTR RNA sequence for each target cell type in a plurality of target cell types, and/or
a delta activity of the respective UTR RNA sequence for each target cell type in the plurality of target cell types, wherein the delta activity is defined as difference between the predicted cell-type-specific activity specific to the respective target cell type and an average activity across the plurality of target cell types;
D) generating, for each respective UTR RNA sequence in the plurality of UTR RNA sequences generated in step B), a predicted set of metrics for the cell-type-specific activity and/or the delta activity for each target cell type in the plurality of target cell types;
E) selecting a sub-plurality of UTR RNA sequences from the plurality of UTR RNA sequences generated in step B), wherein the corresponding predicted sets of metrics for the sub-plurality of UTR RNA sequences satisfy the one or more predefined activity and the one or more cell type-specificity constraints; and
F) outputting the selected sub-plurality of UTR RNA sequences.

15. The method of claim 14, wherein the method further comprises:
evaluating each respective UTR RNA sequence in the plurality of UTR RNA sequences generated to calculate a cell type activity difference (CTAD) score, wherein the CTAD score quantifies the difference in predicted activity of a UTR RNA sequence between two target cell types, and
selecting UTR RNA sequences that maximize the CTAD score while satisfying the one or more predefined activity and the one or more cell type-specificity constraints.

16. The method of claim 14, wherein the model comprises a cold diffusion model, a genetic algorithm, a random sampling model, or a combination thereof.

17. An UTR RNA sequence having cell-type-specific activity, wherein the UTR RNA sequence is obtained according to the method of claim 1.

18. The UTR RNA sequence of claim 17, wherein the UTR RNA sequence has cell-type-specific activity in blood tissue, colon tissue, ovarian tissue, breast tissue, liver tissue, or a combination thereof.

19. A pharmaceutical composition comprising an expression construct, wherein the expression construct comprises the UTR RNA sequence that has cell-type-specific activity specific to a target cell type and the UTR RNA sequence is obtained according to the method of claim 1, and a target gene, wherein the UTR RNA sequence is operably linked to the target gene.

20. A method for regulating expression of a target gene in a target cell, comprising administering the composition of claim 19 to the target cell, and inducing expression of the target gene in the target cell under suitable conditions.
